(19) United States
(12) Patent Application Publication
(10) Pub. No.: US 2024/0041832 A1
(43) Pub. Date: Feb. 8, 2024

(54) METHODS AND COMPOSITIONS FOR TREATING EYE DISEASES

(71) Applicant: JENIVISION INC., Irvine, CA (US)

(72) Inventors: Weizhen WANG, Irvine, CA (US); David F. WOODWARD, Lake Forest, CA (US); Nonna SNIDER, Lake Forest, CA (US)

(21) Appl. No.: 18/550,907

(22) PCT Filed: Mar. 17, 2022
(86) PCT No.: PCT/US2022/020803

§ 371 Date: Sep. 15, 2023

Related U.S. Application Data
(60) Provisional application No. 63/162,960, filed on Mar. 18, 2021.
(60) Provisional application No. 63/219,336, filed on Jul. 7, 2021.
(60) Provisional application No. 63/283,117, filed on Nov. 24, 2021.

(51) Int. Cl. A61K 31/4168; A61K 45/06; A61K 47/14; A61K 47/44; A61K 9/00; A61K 31/202

(52) U.S. Cl. CPC A61K 31/4168; A61K 45/06; A61K 47/14; A61K 47/44; A61K 9/0048; A61K 31/202

(57) ABSTRACT

Described herein are compositions and methods for the topical administration of various compounds to the eye, in particular to the posterior portion of the eye. Such compositions and methods are useful for treating various disease and disorders, as well as in promoting the general health of the eye.

### CROSS-REFERENCE

This application claims the benefit of U.S. Provisional Application No. 63/162,960, filed Mar. 18, 2021, U.S. Provisional Application No. 63/219,336, filed Jul. 7, 2021, and U.S. Provisional Application No. 63/283,117, filed Nov. 24, 2021, each of which is entirely incorporated herein by reference.

### FIELD OF THE INVENTION

Embodiments herein are directed towards the treatment of ocular diseases and delivery of compounds to the eye.

### SUMMARY OF THE INVENTION

In one aspect, provided herein, is a method of treating a disease or disorder of the eye in a patient suffering from the disease or disorder comprising administering a therapeutically effective amount of 4,5-dihydro-N-[4-[[4-(1-methylethoxy) phenyl] methyl] phenyl]-1H-imadazol-2-amine, or a pharmaceutically acceptable ester or salt thereof, to the periorbital skin of an eye of the patient.

In another aspect, provided herein, is a method of treating a disease or disorder of the eye in a patient suffering from the disease or disorder comprising administering a therapeutically effective amount of 4,5-dihydro-N-[4-[[4-(1-methylethoxy) phenyl] methyl] phenyl]-1H-imadazol-2-amine, or a pharmaceutically acceptable ester or salt thereof, to the exterior skin of the eyelid of an eye of the patient.

In a further aspect, provided herein, is a method of treating a disease or disorder of the eye in a patient suffering from the disease or disorder comprising administering a therapeutically effective amount of a compound or mixture with anti-inflammation, anti-oxidation, anti-microvascular leakage, or anti-neovascularization properties, to the exterior skin of the eyelid of an eye on the patient. In an alternative aspect, provided herein, is a method of treating a disease or disorder of the eye in a patient suffering from the disease or disorder comprising administering a therapeutically effective amount of a combination of two or more compounds or mixtures with anti-inflammation, anti-oxidation, anti-microvascular leakage, or anti-neovascularization properties, to the exterior skin of the eyelid of an eye on the patient.

In an additional aspect, provided herein, is a method of treating a disease or disorder of the eye in a patient suffering from the disease or disorder comprising administering a therapeutically effective amount of a compound or mixture with anti-inflammation, anti-oxidation, anti-microvascular leakage, or anti-neovascularization properties, to the periorbital skin of an eye on the patient.

In some embodiments, the compound or mixture comprises docosahexaenoic acid (DHA) for its anti-oxidation, anti-inflammation, neuroprotection, analgesic, and suppression of vasogenic properties. In some embodiments, the compound or mixture comprises omega-3 fatty acids for their anti-oxidation, anti-inflammation, neuroprotection, analgesic, and suppression of vasogenic properties. In some embodiments, the compound or mixture comprises omega-3 fatty acid ethyl esters for their anti-oxidation, anti-inflammation, neuroprotection, analgesic, and suppression of vasogenic properties. In some embodiments, the compound or mixture comprises omega-3 triglycerides for their anti-oxidation, anti-inflammation, neuroprotection, analgesic, and suppression of vasogenic properties. In some embodiments, the compound or mixture comprises omega-3 phospholipids, such as lysophosphatidylcholine (LPC)-omega-3 (LPC-DHA or LPC-EPA) and di-DHA phosphatidylcholine (PC), etc., for their anti-oxidation, anti-inflammation, neuroprotection, analgesic, and suppression of vasogenic properties. In some embodiments, the compound or mixture comprises metabolites of omega-3 fatty, such as leukotriene B₅, leukotriene C₅, leukotriene E₅, prostaglandin E₃, prostaglandin I₃, thromboxane A₃, protectins, maresins, and resolvins. In some embodiments the compound or mixture comprises a compound having the following general structure with a difluoro biphenyl moiety:

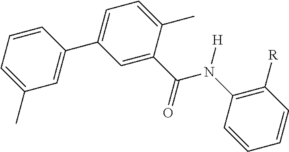

wherein R may be selected from the group consisting of:

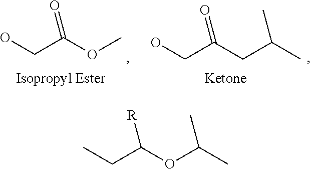

wherein R may be Cl or Br, CF3, alkyl and H;

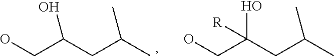

wherein R is methyl, isobutyl,

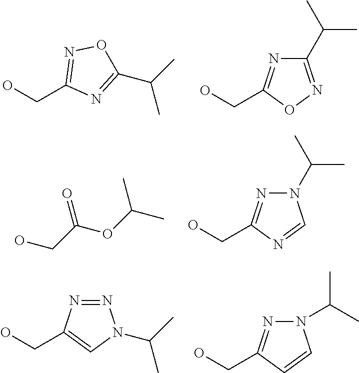

In some embodiments, the compound or mixture comprises a compound selected from the group:

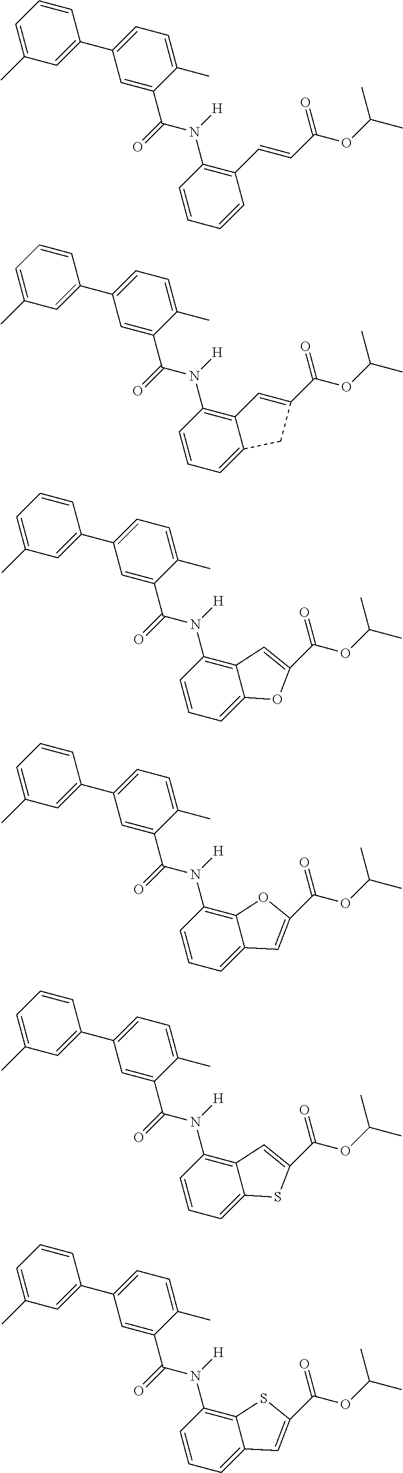

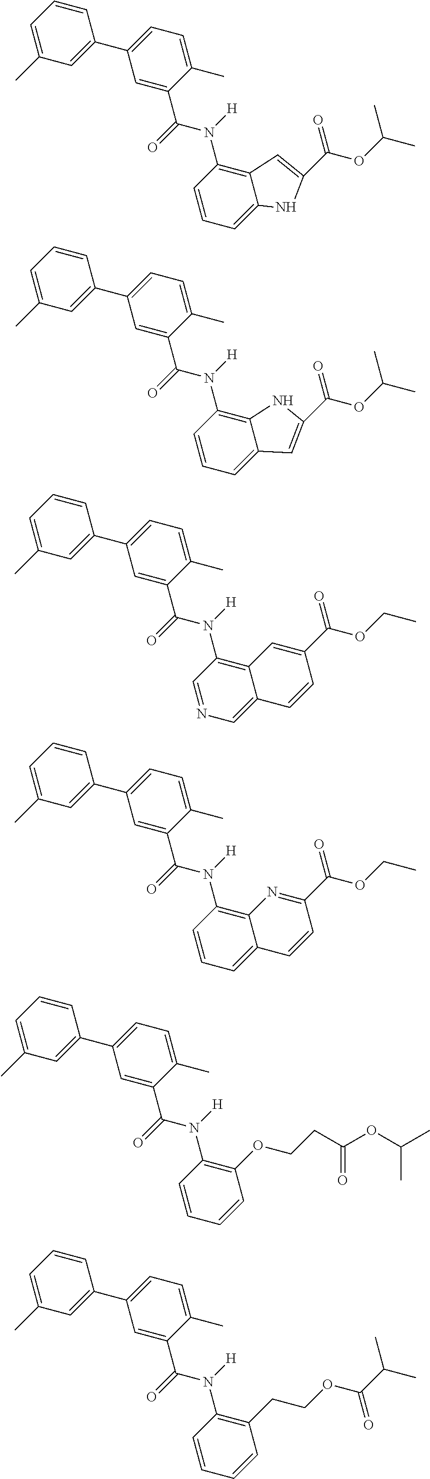

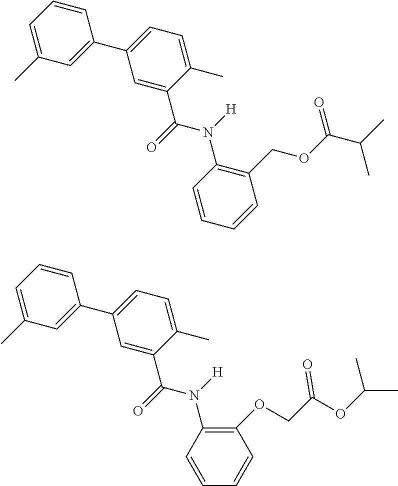

3-[(3′-fluoro-4-fluorobiphenyl-3-carbonyl) amino]phenoxyacetic acid isopropyl ester, its free base, alcaftadine, cromolyn, dexamethasone, brimonidine, difluprednate, fluorometholone, loteprednol, rimexolone, azelastine, epinastine, emedastine difumarate, olopatadine, cromolyn ophthalmic, lodoxamide, nedocromil, bromfenac, diclofenac, flurbiprofen, ketorolac, nepafenac, loradatine, hydroxyzine, diphenhydramine, chlorpheniramine, azelastine hydrochloride brompheniramine, cyproheptadine, terfenadine, clemastine, levocabastine, triprolidine, carbinoxamine, diphenylpyraline, phenindamine, azatadine, tripelennamine, dexchlorpheniramine, dexbrompheniramine, methdilazine, and trimprazine doxylamine, pheniramine, pyrilamine, pemirolast, chiorcyclizine, thonzylamine, cisplatin, etoposide, interferons, camptothecin and derivatives thereof, phenesterine, taxol and derivatives thereof, taxotere and derivatives thereof, vinblastine, vincristine, tamoxifen, etoposide, piposulfan, cyclophosphamide, flutamide, adriamycin, cyclophosphamide, actinomycin, bleomycin, duanorubicin, doxorubicin, epirubicin, mitomycin, methotrexate, fluorouracil, carboplatin, carmustine (BCNU), methyl-CCNU, ampicillin, amoxicillin, cyclacillin, ampicillin, cefazolin, cephradine, cefaclor, cephapirin, ceftizoxime, cefoperazone, cefotetan, cefutoxime, cefotaxime, cefadroxil, ceftazidime, cephalexin, cephalothin, cefamandole, cefoxitin, cefonicid, ceforanide, ceftriaxone, cefadroxil, cephradine, cefuroxime, cyclosporine, gentamicin, tobramycin, besifloxacin, ciprofloxacin, gatifloxacin, levofloxacin, moxifloxacin, ofloxacin, azithromycin, erythromycin, bacitracin, bacitracin/polymyxin, natamycin, neomycin/polymyxinfB/bacitracin, neomycin/polymyxin B/gramicidin, polymyxin B/trimethoprim, penicillin G, penicillin V potassium, piperacillin, oxacillin, bacampicillin, cloxacillin, ticarcillin, azlocillin, carbenicillin, methicillin, nafcillin, erythromycin, tetracycline, doxycycline, minocycline, aztreonam, chloramphenicol, ciprofloxacin hydrochloride, clindamycin, metronidazole, gentamicin, lincomycin, tobramycin, vancomycin, polymyxin B sulfate, colistimethate, colistin, azithromycin, augmentin, sulfamethoxazole, and trimethoprim, and all pharmaceutically acceptable esters or salts, analogs, and isoforms of the preceding compounds, and/or mixtures of at least two thereof.

In some embodiments, the compound or mixture comprises one or more compounds of the following formula:

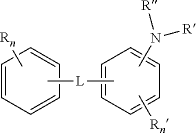

wherein, L is a linker group, each linker group being independently selected from the group consisting of alkyl, cycloalkyl, heteroalkyl, heterocycloalkyl, or alkoxy, Rₙ are each independently selected from the group consisting of hydrogen, halogens, —OH, alkyl, alkoxy, Rₙ′ are each independently selected from the group consisting of hydrogen, halogens, —OH, alkyl, cycloalkyl, heteroalkyl, heterocycloalkyl or alkoxy, R′ and R″ are each independently selected from the group consisting of hydrogen, halogens, —OH, alkyl, cycloalkyl, heteroalkyl, heterocycloalkyl, or alkoxy.

In some embodiments, the compound or mixture comprises one or more compounds of the following formula:

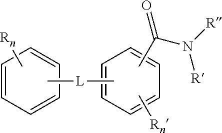

wherein, L is a linker group, each linker group being independently selected from the group consisting of alkyl, cycloalkyl, heteroalkyl, or alkoxy, Rₙ are each independently selected from the group consisting of hydrogen, halogens, —OH, alkyl, alkoxy, Rₙ′ are each independently selected from the group consisting of hydrogen, halogens, —OH, alkyl, cycloalkyl, heteroalkyl, or alkoxy, R′ and R″ are each independently selected from the group consisting of hydrogen, halogens, —OH, alkyl, cycloalkyl, heteroalkyl, or alkoxy.

In some embodiments, the compound or mixture is selected the group consisting of the following families of compounds: C-C motif receptor 3 (CCR3) inhibitors, vitamin A and modified forms of vitamin A (such as NCT03845582), complement factor 1q inhibitors, Apurinic/Apyrimidinic Endonuclease 1/Redox Effector Factor-1 (APE1/Ref-1) inhibitors, Steroids, endothelial cell (EC)-specific receptor tyrosine kinases Tie2 agonists (activators), angiopoietin-2 antagonists, Retinol-binding protein 4 (RBP4) antagonists, Complement component 3 (C3) inhibitors, pan-arginylglycylaspartic acid (RGD) integrin antagonists, connexin43 hemichannels blockers, Complement component 5 inhibitors, pan RGD integrin antagonists, Rho kinase inhibitors, Ref-1 inhibitors, AP endonuclease 1 inhibitors, serine/threonine-protein kinase (SRPK1) inhibitors, CC3 (TIP30=tat-interacting protein of 30 kDa) inhibitors, Complement C1Q Inhibitors, Compliment factor (B, and D) inhibitors, C3 convertase inhibitors, C5 convertase inhibitors, Inflammasome inhibitors, HtrA1 inhibitors, Matrix modulators (such as doxycycline), MASP2 (MBL Associated Serine Protease 2) blockers, MASP3 (MBL Associated Serine Protease 3) blockers, Antiviral drugs (such as Ganciclovir), VEGF receptor (R1, R2, R3) inhibitors, PDGF receptor inhibitors (including imatinib, sorafenib, and sunitinib), Prostanoid IP receptor antagonists, tyrosine kinases inhibitors, PAF (platelet-activating factor) receptor inhibitors, and combinations of two or more thereof.

In some embodiments, the disease or disorder is of the posterior of the eye. In some embodiments, the disease or disorder is of the anterior of the eye. In some embodiments, wherein the disease or disorder of the posterior of the eye comprises a retinal disease. In some embodiments, the disease or disorder is an anterior segment eye disease (ASED). In some embodiments the disease or disorder is a posterior segment eye disease (PSED). In some embodiments, the disease or disorder is dry eye disease and ocular discomfort, irritation, pain and stress, chemical burns, anterior segment dysgenesis, cataract, iritis, pterygium, keratoconjunctivitis, keratitis, conjunctivitis, keratoconus, ectatic disorders (including keratoglobus, pellucid marginal degeneration), Pseudophakic and aphakic bullous keratopathy, episcleritis, corneal ulceration, corneal dysplasia, corneal ulceration, Fuchs' endothelial dystrophy and other corneal dystrophies (including lattice, granular, macular, andmap-dot fingerprint), ocular cicatricial pemphigoid, Stevens Johnson syndrome, acute and chronic uveitis (anterior uveitis, intermediate uveitis), trauma to the cornea, conjunctiva and anterior segment including iris trauma, penetrating ocular trauma, blepharitis, blepharospasm, chalazion, ptosis, coloboma, dermatochalasis, ectropion, entropion, trichiasis, stye, meibomianitis, Meibomian Gland Dysfunction, lacrimal gland obstruction, lacrimal gland obstruction, seborrheic keratitis, actinic keratitis, bacterial infection, or viral infection, Refractive errors (myopia, hyperopia, and astigmatism), Uveitis (including acute and chronic Uveitis of anterior-, intermediate-, posterior, -and pan-uveitis), age-related macular degeneration (wet and dry forms), dry and wet macular degeneration, lattice Degeneration, macular hole, macular pucker, lattice degeneration, retinal tear, retinal detachment, retinal artery occlusion, retinal vein occlusion, central retinal vein occlusion, intraocular tumors, pediatric, neonatal or Inherited retinal disorders, hereditary retinal dystrophies, geographic atrophy, retinitis pigmentosa (including Leber congenital amaurosis), cytomegalovirus (cmv) retinal infection, infectious retinitis, retinoblastoma, endophthalmitis, chorioretinitis, myopic macular degeneration, and normal-tension glaucoma, retinal degeneration in glaucoma; various retinopathies, including but not limited to diabetic retinopathy, retinopathy of prematurity, Sickel cell retinopathy, radiation/solar retinopathy, central serous retinopathy, hypertensive retinopathy, peripheral retinopathy and neuropathy; macular edema, retinal hemorrhage, diabetic macular edema, diabetic macularischemia, geographic atrophy, Stargardt disease, lymphatic malformations of the orbit (a.k.a. orbital lymphangiomas), or thyroid eye disease (Graves' Eye Disease), or combinations of two or more thereof.

In some embodiments, wherein the disease or disorder of the posterior of the eye comprises a retinal disease, the retinal disease further comprises hemorrhage from the retinal or choroidal vasculature. In some embodiments, the hemorrhage is caused by hypertension, diabetes, fatty liver disease, obesity, shaken baby syndrome, head trauma, anemia, or leukemia.

In some embodiments, the retinal disease or disorder comprises plasmaleakage from the retinal or choroidal vasculature. In some embodiments, the plasma leakage is caused by systemic hypertension, diabetes, fatty liver disease, obesity, shaken baby syndrome, head trauma, anemia, or leukemia.

In some embodiments, the retinal disease or disorder comprises macular edema formation involving the retinal or choroidal vasculature.

In some embodiments, the retinal disease or disorder is age-related macular degeneration (wet and dry forms), macular hole, macular pucker, lattice degeneration, retinal tear, retinal detachment, retinal artery occlusion, branch retinal vein occlusion, central retinal vein occlusion, intraocular tumors, pediatric and neonatal retinal disorders, hereditary retinal dystrophies, geographic atrophy, retinitis pigmentosa (including Leber congenital amaurosis), cytomegalovirus (cmv) retinal infection, infectious retinitis, central serous retinopathy, retinoblastoma, diabetic macular edema (DME), retinal vein occlusion, or endophthalmitis.

In some embodiments, the retinal disease or disorder is age-related macular degeneration.

In some embodiments, the disease or disorder of the posterior of the eye is posterior uveitis.

In some embodiments, the method further comprises administering to the patient an additional therapeutic agent. In some embodiments, the additional therapeutic agent is a VEGF antibody, a PDGF antibody, a FGF antibody, a SDF-1 antibody, a HIF-1 antibody, a PIGF antibody, a TNF-alpha antibody, an IGF-1 antibody, a VEGF receptor antagonist, a PDGF receptor antagonist, a FGF receptor antagonist, a SDF-1 receptor antagonist, a HIF-1 receptor antagonist, a PIGF receptor antagonist, a TNF-alpha receptor antagonist, a IGF-1 receptor antagonist, a tyrosine kinase inhibitor, a steroidal anti-inflammatory agent, a non-steroidal anti-inflammatory agent, an immunosuppressant, an anti-cholinergic agent, thalidomide, a prostaglandin receptor antagonist, a neuroprotective agent, a neuro-regenerative agent, an RNA interference molecule that provides neuroprotection, an RNA interference molecule that promotes neuro-regeneration, a small molecule that directly provides neuroprotection and reduces intraocular pressure, an RNA interference molecule that promotes neuro-regeneration and reduces intraocular pressure, an RNA interference that provides neuroprotection and reduces intraocular pressure, an antibody that reduces edema, hemorrhage, and angiogenesis, an RNA interference that reduces edema, hemorrhage and angiogenesis.

In some embodiments drug application to the periorbital skin would be the preferred delivery route to the sclera for the prevention of the increase in the long axis of the globe resulting from facilitating alterations to the sclera.

In some embodiments, drug delivery may involve nanoparticles. These may be selected from a group comprising polymeric, lipid based, liposomes, albumin bound, inorganic, organic crystals, and viral based nanoparticles

In some embodiments, the 4,5-dihydro-N-[4-[[4-(1-methylethoxy) phenyl] methyl]phenyl]-1H-imadazol-2-amine is administered prophylactically, as an emergency intervention, or as required to achieve the desired remedial effects.

In some embodiments, the 4,5-dihydro-N-[4-[[4-(1-methylethoxy) phenyl] methyl]phenyl]-1H-imadazol-2-amine is administered as a composition. In some embodiments, the 4,5-dihydro-N-[4-[[4-(1-methylethoxy) phenyl] methyl] phenyl]-1H-imadazol-2-amine is present in an amount of about 0.0001% to about 10% (w/w) of the composition.

In some embodiments, the composition is an aqueous solution, a non-aqueous solution, an oil solution, a gel, a suspension, an emulsion, a lotion, a cream, or an ointment. In some embodiments, the composition is an ointment. In some embodiments, the ointment comprises petrolatum, beeswax, or cocoa butter. In some embodiments, the ointment comprises petrolatum and medium-chain triglycerides. In some embodiments, the medium-chain triglycerides comprise a mixture of C6, C8, C10 and C12 fatty acids. In some embodiments, the medium-chain triglycerides comprise a mixture of caprylic acid and capric acid. In some embodiments, the ointment comprises petrolatum and medium-chain triglyceride in the ratio of about 1:1 (v/v), about 2:1 (v/v), about 3:1 (v/v), about 4:1 (v/v), about 5:1 (v/v), or about 6:1 (v/v). In some embodiments, the ointment comprises petrolatum and medium-chain triglyceride in the ratio of about 4:1 (v/v).

In some embodiments, the molecular weight of the composition is about 50 Da to about 500 Da. In some embodiments, the molecular weight of the composition is about 50 Da to about 100 Da, about 50 Da to about 150 Da, about 50 Da to about 200 Da, about 50 Da to about 250 Da, about 50 Da to about 300 Da, about 50 Da to about 350 Da, about 50 Da to about 400 Da, about 50 Da to about 450 Da, about 50 Da to about 500 Da, about 100 Da to about 150 Da, about 100 Da to about 200 Da, about 100 Da to about 250 Da, about 100 Da to about 300 Da, about 100 Da to about 350 Da, about 100 Da to about 400 Da, about 100 Da to about 450 Da, about 100 Da to about 500 Da, about 150 Da to about 200 Da, about 150 Da to about 250 Da, about 150 Da to about 300 Da, about 150 Da to about 350 Da, about 150 Da to about 400 Da, about 150 Da to about 450 Da, about 150 Da to about 500 Da, about 200 Da to about 250 Da, about 200 Da to about 300 Da, about 200 Da to about 350 Da, about 200 Da to about 400 Da, about 200 Da to about 450 Da, about 200 Da to about 500 Da, about 250 Da to about 300 Da, about 250 Da to about 350 Da, about 250 Da to about 400 Da, about 250 Da to about 450 Da, about 250 Da to about 500 Da, about 300 Da to about 350 Da, about 300 Da to about 400 Da, about 3001 Da to about 450 Da, about 300 Da to about 500 Da, about 350 Da to about 400 Da, about 3501 Da to about 450 Da, about 350 Da to about 500 Da, about 400 Da to about 450 Da, about 400 Da to about 500 Da, or about 450 Da to about 5001 Da. In some embodiments, the molecular weight of the composition is about 50 Da, about 100 Da, about 1501 Da, about 200 Da, about 250 Da, about 300 Da, about 350 Da, about 4001 Da, about 450 Da, or about 500 Da. In some embodiments, the molecular weight of the composition is at least about 50 Da, about 100 Da, about 150 Da, about 200 Da, about 250 Da, about 300 Da, about 350 Da, about 400 Da, or about 450 Da. In some embodiments, the molecular weight of the composition is at most about 100 Da, about 150 Da, about 200 Da, about 250 Da, about 300 Da, about 350 Da, about 400 Da, about 450 Da, or about 500 Da.

In some embodiments, the composition comprises an antibody. In some embodiments, the composition has a molecular weight of around 150 kDa. In some embodiments, the composition has a molecular weight of about 7 kDa. In some embodiments, the composition comprises a double stranded siRNA. In some embodiments, the composition comprises a single stranded siRNA. In some embodiments, the composition comprises a short oligo peptide.

In some embodiments, the short oligo peptide is a sequence of about 1 amino acid to about 6 amino acids. In some embodiments, the short oligo peptide is a sequence of about 1 amino acid to about 2 amino acids, about 1 amino acid to about 3 amino acids, about 1 amino acid to about 4 amino acids, about 1 amino acid to about 5 amino acids, about 1 amino acid to about 6 amino acids, about 2 amino acids to about 3 amino acids, about 2 amino acids to about 4 amino acids, about 2 amino acids to about 5 amino acids, about 2 amino acids to about 6 amino acids, about 3 amino acids to about 4 amino acids, about 3 amino acids to about 5 amino acids, about 3 amino acids to about 6 amino acids, about 4 amino acids to about 5 amino acids, about 4 amino acids to about 6 amino acids, or about 5 amino acids to about 6 amino acids. In some embodiments, the short oligo peptide is a sequence of about 1 amino acid, about 2 amino acids, about 3 amino acids, about 4 amino acids, about 5 amino acids, or about 6 amino acids. In some embodiments, the short oligo peptide is a sequence of at least about 1 amino acid, about 2 amino acids, about 3 amino acids, about 4 amino acids, or about 5 amino acids. In some embodiments, the short oligo peptide is a sequence of at most about 2 amino acids, about 3 amino acids, about 4 amino acids, about 5 amino acids, or about 6 amino acids.

In some embodiments the composition comprises liposomes. In some embodiments the composition comprises nanoparticles. In some embodiments, the average diameter of the liposomes or nanoparticles is about 0.1 μm to about 1 μm. In some embodiments, the average diameter of the liposomes or nanoparticles is about 0.1 μm to about 0.2 μm, about 0.1 μm to about 0.3 μm, about 0.1 μm to about 0.4 μm, about 0.1 μm to about 0.5 μm, about 0.1 μm to about 0.6 μm, about 0.1 μm to about 0.7 μm, about 0.1 μm to about 0.8 μm, about 0.1 μm to about 0.9 μm, about 0.1 μm to about 1 μm, about 0.2 μm to about 0.3 μm, about 0.2 μm to about 0.4 μm, about 0.2 μm to about 0.5 μm, about 0.2 μm to about 0.6 μm, about 0.2 μm to about 0.7 μm, about 0.2 μm to about 0.8 μm, about 0.2 μm to about 0.9 μm, about 0.2 μm to about 1 μm, about 0.3 μm to about 0.4 μm, about 0.3 μm to about 0.5 μm, about 0.3 μm to about 0.6 μm, about 0.3 μm to about 0.7 μm, about 0.3 μm to about 0.8 μm, about 0.3 μm to about 0.9 μm, about 0.3 μm to about 1 μm, about 0.4 μm to about 0.5 μm, about 0.4 μm to about 0.6 μm, about 0.4 μm to about 0.7 μm, about 0.4 μm to about 0.8 μm, about 0.4 μm to about 0.9 μm, about 0.4 μm to about 1 μm, about 0.5 μm to about 0.6 μm, about 0.5 μm to about 0.7 μm, about 0.5 μm to about 0.8 μm, about 0.5 μm to about 0.9 μm, about 0.5 μm to about 1 μm, about 0.6 μm to about 0.7 μm, about 0.6 μm to about 0.8 μm, about 0.6 μm to about 0.9 μm, about 0.6 μm to about 1 μm, about 0.7 μm to about 0.8 μm, about 0.7 μm to about 0.9 μm, about 0.7 μm to about 1 μm, about 0.8 μm to about 0.9 μm, about 0.8 μm to about 1 μm, or about 0.9 μm to about 1 μm. In some embodiments, the average diameter of the liposomes or nanoparticles is about 0.1 μm, about 0.2 μm, about 0.3 μm, about 0.4 μm, about 0.5 μm, about 0.6 μm, about 0.7 μm, about 0.8 μm, about 0.9 μm, or about 1 μm. In some embodiments, the average diameter of the liposomes or nanoparticles is at least about 0.1 μm, about 0.2 μm, about 0.3 μm, about 0.4 μm, about 0.5 μm, about 0.6 μm, about 0.7 μm, about 0.8 μm, or about 0.9 μm. In some embodiments, the average diameter of the liposomes or nanoparticles is at most about 0.2 μm, about 0.3 μm, about 0.4 μm, about 0.5 μm, about 0.6 μm, about 0.7 μm, about 0.8 μm, about 0.9 μm, or about 1 μm.

In some embodiments, the composition comprises a lipophilic compound. In some embodiments, the composition comprises a nonpolar compound. In some embodiments, the composition comprises a bipolar compound. In some embodiments, the composition comprises a zwitterion.

In some embodiments, the composition is an aqueous solution. In some embodiments, the aqueous solution comprises a polyoxyl castor oil. In some embodiments, the polyoxyl castor oil is a polyethylene glycol (PEG)-ylated castor oil. In some embodiments, the polyoxyl castor oil is polyoxyl 35 castor oil. In some embodiments, the polyoxyl 35 castor oil is present in an amount of about 0.1% to about 5%, about 0.1% to about 10%, about 0.1% to about 15%, or about 0.1% to about 20% (w/w) of the composition. In some embodiments, the composition comprises an ocular surface lubricating agent.

In some embodiments, the 4,5-dihydro-N-[4-[[4-(1-methylethoxy) phenyl] methyl]phenyl]-1H-imadazol-2-amine is applied to the periorbital skin of at least one eye of the patient by dropper, pump, spray, click pen or roller/reservoir device. In some embodiments, the 4,5-dihydro-N-[4-[[4-(1-methylethoxy) phenyl] methyl] phenyl]-1H-imadazol-2-amine is applied to the periorbital skin of at least one eye of the patient by brush, Q-tip, fingertip or spatula and where the application process is optionally preceded by using a graduated dropper, syringe, click pen or pipette.

In some embodiments periorbital skin penetration may be assisted by a penetration enhancer, tape-stripping, microdermabrasion, solvent, pulsed laser, and iontophoresis for delivering macromolecules such as antibodies, siRNA, in liposomes or nanoparticles. In some embodiments, the composition is readily capable of penetrating the skin barrier.

In some embodiments 4,5-dihydro-N-[4-[[4-(1-methylethoxy) phenyl] methyl] phenyl]-1H-imadazol-2-amine is administered to the periorbital skin of each eye of the patient four times per day, three times per day, twice per day, once per day, once every other day, once every three days, once every four days, or once every seven days. In some embodiments, the 4,5-dihydro-N-[4-[[4-(1-methylethoxy) phenyl] methyl] phenyl]-1H-imadazol-2-amine is administered once per day. In some embodiments, the method comprises administering the composition to the periorbital skin above the upper eyelid, below the lower eyelid, or both above the upper and below the lower eyelids.

In one aspect, provided herein, is a method of treating a disease or disorder of the posterior of the eye in a patient suffering from the disease or disorder comprising administering a therapeutically effective amount of 4,5-dihydro-N-[4-[[4-(1-methylethoxy) phenyl] methyl]phenyl]-1H-imadazol-2-amine, or a pharmaceutically acceptable ester or salt thereof, to the ocular surface of an eye of the patient.

In some embodiments, the disease or disorder of the posterior of the eye comprises a retinal disease.

In some embodiments, the retinal disease comprises hemorrhage from the retinal or choroidal vasculature. In some embodiments, the hemorrhage is caused by hypertension, diabetes, fatty liver disease, obesity, shaken baby syndrome, head trauma, anemia, or leukemia.

In some embodiments, the retinal disease or disorder comprises plasma leakage from the retinal or choroidal vasculature. In some embodiments, the plasma leakage is caused by systemic hypertension, diabetes, fatty liver disease, obesity, shaken baby syndrome, head trauma, anemia, or leukemia.

In some embodiments, the retinal disease or disorder comprises macular edema formation involving the retinal or choroidal vasculature.

In some embodiments, the retinal disease or disorder is age-related macular degeneration (wet and dry forms), macular hole, macular pucker, lattice degeneration, retinal tear, retinal detachment, retinal artery occlusion, branch retinal vein occlusion, central retinal vein occlusion, intraocular tumors, pediatric and neonatal retinal disorders, hereditary retinal dystrophies, geographic atrophy, retinitis pigmentosa (including Leber congenital amaurosis), cytomegalovirus (cmv) retinal infection, infectious retinitis, central serous retinopathy, retinoblastoma, and endophthalmitis.

In some embodiments, the retinal disease or disorder is age-related macular degeneration.

In some embodiments, the disease or disorder of the posterior of the eye is posterior uveitis.

In some embodiments, the disease is a neurodegenerative ocular disease.

In some embodiments, the method further comprises administering to the patient an additional therapeutic agent. In some embodiments, the additional therapeutic agent is a VEGF antibody, a PDGF antibody, a bFGF antibody, a SDF-1 antibody, a HIF-1 antibody, a PIGF antibody, a VEGF antagonist, a tyrosine kinase inhibitor, a steroidal anti-inflammatory agent, a non-steroidal anti-inflammatory agent, an immunosuppressant, an anti-cholinergic agent, thalidomide, a prostaglandin receptor antagonist, or a neuroprotective agent, an antiangiogenic factor (e.g. PEDF), a neuro-regenerative agent, RNA interference that provides neuroprotection, RNA interference that promotes neuro-regeneration, a small molecule that directly provides neuroprotection and reduces intraocular pressure, RNA interference that promotes neuro-regeneration and reduces intraocular pressure, RNA interference that provides neuroprotection and reduces intraocular pressure, an antibody that reduces edema, hemorrhage, and angiogenesis, RNA interference that reduces edema, hemorrhage and angiogenesis.

In some embodiments, the 4,5-dihydro-N-[4-[[4-(1-methylethoxy) phenyl] methyl]phenyl]-1H-imadazol-2-amine is administered prophylactically, as an emergency intervention, or as required to achieve the desired remedial effects.

In some embodiments, the 4,5-dihydro-N-[4-[[4-(1-methylethoxy) phenyl] methyl]phenyl]-1H-imadazol-2-amine is administered as a composition. In some embodiments, the composition is an aqueous solution, a non-aqueous solution, an oil solution, a gel, a suspension, an emulsion, a cream, or an ointment. In some embodiments, the 4,5-dihydro-N-[4-[[4-(1-methylethoxy) phenyl] methyl] phenyl]-1H-imadazol-2-amine is present in an amount of about 0.0001% to about 10% (w/w) of the composition.

In some embodiments, the composition is an aqueous solution. In some embodiments, the aqueous solution comprises a polyoxyl castor oil. In some embodiments, the polyoxyl castor oil is a polyethylene glycol (PEG)-ylated castor oil. In some embodiments, the polyoxyl castor oil is polyoxyl 35 castor oil. In some embodiments, the polyoxyl 35 castor oil is present in an amount of about 0.1% to about 5%, about 0.1% to about 10%, about 0.1% to about 15%, or about 0.1% to about 20% (w/w) of the composition. In some embodiments, the composition comprises an ocular surface lubricating agent.

In some embodiments, the 4,5-dihydro-N-[4-[[4-(1-methylethoxy) phenyl] methyl]phenyl]-1H-imadazol-2-amine is administered to the ocular surface of each eye of the patient four times per day, three times per day, twice per day, once per day, once every other day, once every three days, once every four days, or once every seven days. In some embodiments, the 4,5-dihydro-N-[4-[[4-(1-methylethoxy) phenyl] methyl] phenyl]-1H-imadazol-2-amine is administered once per day.

In one aspect, provided herein, is a method of treating uveitis in a patient suffering from uveitis comprising administering to the eye of the patient a therapeutically effective amount of 4,5-dihydro-N-[4-[[4-(1-methylethoxy) phenyl] methyl] phenyl]-1H-imadazol-2-amine or a pharmaceutically acceptable ester or salt thereof.

In one aspect, provided herein, is a method of treating pterygium in a patient suffering from pterygium comprising administering to the eye of the patient a therapeutically effective amount of 4,5-dihydro-N-[4-[[4-(1-methylethoxy) phenyl] methyl] phenyl]-1H-imadazol-2-amine or a pharmaceutically acceptable ester or salt thereof.

In one aspect, provided herein, is a method of treating an ocular disease or disorder in a patient suffering from the disease or disorder, comprising administering to the eye of the patient a therapeutically effective amount of 4,5-dihydro-N-[4-[[4-(1-methylethoxy) phenyl] methyl]phenyl]-1H-imadazol-2-amine or a pharmaceutically acceptable ester or salt thereof, wherein the ocular disease or disorder is keratoconjunctivitis, keratoconus, episcleritis, corneal ulceration, corneal dysplasia, corneal dystrophy, or Stevens Johnson syndrome.

In one aspect, provided herein, is a method of treating an ocular disease or disorder affecting the eyelid of a patient suffering from the disease or disorder, comprising administering to the eye of the patient a therapeutically effective amount of 4,5-dihydro-N-[4-[[4-(1-methylethoxy) phenyl] methyl] phenyl]-1H-imadazol-2-amine or a pharmaceutically acceptable ester or salt thereof, wherein the ocular disease or disorder affecting the eyelid is blepharitis, blepharospasm, ptosis, coloboma, dermatochalasis, ectropion, entropion, trichiasis, stye, chalaziion, meibomianitis, lacrimal gland obstruction, lacrimal gland obstruction, seborrheic keratitis, actinic keratitis, bacterial infection, or viral infection.

In some embodiments, the 4,5-dihydro-N-[4-[[4-(1-methylethoxy) phenyl] methyl]phenyl]-1H-imadazol-2-amine is administered topically to the surface of the eye as a composition.

In some embodiments, the 4,5-dihydro-N-[4-[[4-(1-methylethoxy) phenyl] methyl]phenyl]-1H-imadazol-2-amine is administered periorbitally as a composition.

In some embodiments, the composition is an aqueous solution, a non-aqueous solution, an oil solution, a gel, a suspension, an emulsion, a cream, or an ointment. In some embodiments, the composition is an aqueous solution. In some embodiments, the aqueous solution comprises a polyoxyl castor oil. In some embodiments, the polyoxyl castor oil is a polyethylene glycol (PEG)-ylated castor oil. In some embodiments, the polyoxyl castor oil is polyoxyl 35 castor oil. In some embodiments, the polyoxyl 35 castor oil is present in an amount of about 0.1% to about 5%, about 0.1% to about 10%, about 0.1% to about 15%, or about 0.1% to about 20% (w/w) of the composition. In some embodiments, the composition comprises an ocular surface lubricating agent.

In some embodiments, the 4,5-dihydro-N-[4-[[4-(1-methylethoxy) phenyl] methyl]phenyl]-1H-imadazol-2-amine is topically applied by dropper, pump, spray, click pen or roller/reservoir device. In some embodiments, the 4,5-dihydro-N-[4-[[4-(1-methylethoxy) phenyl] methyl] phenyl]-1H-imadazol-2-amine is topically applied to the periorbital skin of at least one eye by brush, Q-tip, fingertip, or spatula and where the application process may be preceded by using a graduated dropper, syringe, click pen or pipette.

In some embodiments, the 4,5-dihydro-N-[4-[[4-(1-methylethoxy) phenyl] methyl]phenyl]-1H-imadazol-2-amine is administered to the patient four times per day, three times per day, twice per day, once per day, once every other day, once every three days, once every four days, or once every seven days. In some embodiments, the 4,5-dihydro-N-[4-[[4-(1-methylethoxy) phenyl] methyl] phenyl]-1H-imadazol-2-amine is administered once per day.

In one aspect, provided herein, is a pharmaceutical composition suitable for topical periorbital administration, comprising 4,5-dihydro-N-[4-[[4-(1-methylethoxy) phenyl] methyl]phenyl]-1H-imadazol-2-amine.

In some embodiments, the composition is an aqueous solution, a non-aqueous solution, an oil solution, a gel, a suspension, an emulsion, a cream, or an ointment. In some embodiments, composition is formulated as an oil solution. In some embodiments, composition comprises an oil in an amount of about 1% to about 100% (w/w) of the composition. In some embodiments, the composition comprises an oil in an amount of at least about 90%, at least about 91%, at least about 92%, at least about 93%, at least about 94%, at least about 95%, at least about 96%, at least about 97%, at least about 98%, at least about 99%, at least about 99.5%, at least about 99.6%, at least about 99.7%, at least about 99.8%, at least about 99.9%, at least about 99.95%, at least about 99.96%, at least about 99.97%, at least about 99.98%, or at least about 99.99% (w/w) of the composition. In some embodiments, the oil is derived from a natural source. In some embodiments, the oil is derived from plants, plant seeds, or nuts, or any combination thereof. In some embodiments, the oil comprises a medium-chain triglyceride. In some embodiments, the medium-chain triglyceride comprise a mixture of C6, C8, C10 or C12 fatty acids.

In some embodiments, the 4,5-dihydro-N-[4-[[4-(1-methylethoxy) phenyl] methyl]phenyl]-1H-imadazol-2-amine is present in an amount of from about 0.00011% to about 10% (w/w) of the composition. In some embodiments, the pharmaceutical composition is configured to dispense from about 10 ng to about 5 mg of the 4,5-dihydro-N-[4-[[4-(1-methylethoxy) phenyl] methyl] phenyl]-1H-imadazol-2-amine per administration.

In some embodiments, the composition further comprises an emollient, a humectant, a thickening agent, a preservative, a penetration enhancer, or any combination thereof.

In one aspect, provided herein, is a pharmaceutical composition suitable for topical ocular surface administration, comprising 4,5-dihydro-N-[4-[[4-(1-methylethoxy) phenyl] methyl]phenyl]-1H-imadazol-2-amine, or a pharmaceutically acceptable ester or salt thereof, and a polyoxyl castor oil. In some embodiments, the polyoxyl castor oil is a polyethylene glycol (PEG)-ylated castor oil. In some embodiments, the ratio of PEG to castor oil is from about 20:1 to about 50:1. In some embodiments, the polyoxyl castor oil is polyoxyl 35 castor oil. In some embodiments, the polyoxyl 35 castor oil is present in an amount of about 0.1% to about 5%, about 0.1% to about 10%, about 0.1% to about 15%, or about 0.1% to about 20% (w/w) of the composition. In some embodiments, the polyoxyl 35 castor oil is present in an amount of about 1% (w/w) of the composition.

In some embodiments, the composition further comprises an ocular surface lubricating agent. In some embodiments, the ocular surface lubricating agent is polyethylene glycol, propylene glycol, polyvinyl alcohol, castor oil or glycerol. In some embodiments, the ocular surface lubricating agent is present in an amount of about 0.05% to about 2% (w/w) of the composition. In some embodiments, the composition further comprises a buffer. In some embodiments, wherein the pharmaceutical composition has a pH of from about 6.5 to about 8.5.

In some embodiments, the 4,5-dihydro-N-[4-[[4-(1-methylethoxy) phenyl] methyl]phenyl]-1H-imadazol-2-amine is present in an amount of from about 0.00011% to about 10% (w/w) of the composition.

In one aspect, provided herein, is a method of promoting ocular health, preventing, or treat ocular disease in a subject, comprising administering to the periorbital skin of an eye the subject a topical pharmaceutical composition comprising an omega-3 fatty acid, or a pharmaceutically acceptable ester or salt thereof. In some embodiments, the omega-3 fatty acid is isolated from fish tissue. In some embodiments, the omega-3 fatty acid is isolated from krill, a small, shrimp-like crustacean with big black eyes and a reddish, semi-transparent body. In some embodiments, the omega-3 fatty acid is isolated from a plant source. In some embodiments, the omega-3 fatty acid comprises alpha-linolenic acid (ALA), eicosapentaenoic acid (EPA), docosahexaenoic acid (DHA), or any combination thereof. In some embodiments, the omega-3 fatty acid comprises DHA. In some embodiments, the omega-3 fatty acid is administered in an amount of from about 0.01 mg to about 10000 mg, about 0.01 mg to about 3000 mg, about 0.01 mg to about 1000 mg, about 0.01 mg to about 500 mg, or about 0.01 mg to about 100 mg per eye if used as eye pad; about 0.01 mg to about 200 mg, or about 0.01 mg to about 100 mg, about 0.01 mg to about 50 mg, about 0.01 mg to about 25 mg, about 0.01 mg to about 10 mg, about 0.01 mg to about 5 mg, about 0.01 mg to about 1 mg, or about 0.01 mg to about 0.5 mg per eye per application on periorbital skin, if not used as eye pad.

In one aspect, provided herein, is a method of promoting ocular health, preventing or treating ocular disease in a subject, comprising administering to the periorbital skin of an eye the subject a topical pharmaceutical composition comprising an omega-3 fatty ethyl ester, or a pharmaceutically acceptable ester or salt thereof. In some embodiments, the omega-3 fatty ethyl ester is derived from fish tissue. In some embodiments, the omega-3 fatty ethyl ester is derived from a plant source. In some embodiments, the omega-3 fatty acid comprises ethyl alpha-linoleate, ethyl eicosapentaenoate, ethyl docosahexaenoate, or any combination thereof. In some embodiments, the omega-3 fatty ethyl ester is administered in an amount of from about 0.01 mg to about 5000 mg, about 0.01 mg to about 3000 mg, about 0.01 mg to about 1000 mg, about 0.01 mg to about 500 mg, or about 0.01 mg to about 100 mg per eye if used as eye pad; about 0.01 mg to about 200 mg, or about 0.01 mg to about 100 mg, about 0.01 mg to about 50 mg, about 0.01 mg to about 25 mg, about 0.01 mg to about 10 mg, about 0.01 mg to about 5 mg, about 0.01 mg to about 1 mg, or about 0.01 mg to about 0.5 mg per eye per application on periorbital skin, if not used as eye pad.

In some embodiments, the topical pharmaceutical composition is formulated as a cream, emulsion, ointment, or oil solution. In some embodiments, the topical pharmaceutical composition further comprises an emollient, a humectant, a thickening agent, a preservative, a penetration enhancer, an anti-oxidant, an odor masking agent, or any combination thereof. In some embodiments, the compositions further comprises a preservative.

In some embodiments, the topical pharmaceutical composition is administered with a bottle with a roller ball, a click pen brush, a pump bottle, or an eye drop bottle, eye pad and Q-tip.

In some embodiments, promoting ocular health, preventing or treating ocular disease comprises treating or preventing age-related vision loss. In some embodiments, promoting ocular health, preventing or treating ocular disease comprises treating or preventing dry eye. In some embodiments, promoting ocular health, preventing or treating ocular disease comprises treating or preventing age-related macular degeneration. In some embodiments, promoting ocular health, preventing or treating ocular disease comprises treating or preventing dry age-related macular degeneration.

In some embodiments, the topical pharmaceutical composition is administered to the patient four times per day, three times per day, twice per day, once per day, once every other day, once every three days, once every four days, or once every seven days. In some embodiments, the method comprises administering the composition to the periorbital skin above the upper eyelid, below the lower eyelid, or both above the upper and below the lower eyelids.

In some embodiments, administering the composition to the periorbital skin results in a tissue concentration of the omega-3 fatty acid of at least 110 micrograms/gram in the retina of the eye of the subject 30 minutes after administration greater than compared to baseline.

In one aspect, provided herein, is a pharmaceutical composition suitable for topical periorbital administration, comprising an omega-3 fatty acid, or a pharmaceutically acceptable ester or salt thereof, and a pharmaceutically acceptable excipient.

In some embodiments, the omega-3 fatty acid is isolated from fish tissue. In some embodiments, the omega-3 fatty acid is isolated from a plant source. In some embodiments, wherein the omega-3 fatty acid comprises alpha-linolenic acid (ALA), eicosapentaenoic acid (EPA), docosahexaenoic acid (DHA), or any combination thereof. In some embodiments, the omega-3 fatty acid comprises DHA. In some embodiments, the omega-3 fatty acid is present in an amount of about 0.01% to about 100% (w/w) of the composition.

In some embodiments, the composition is formulated as a cream, emulsion, ointment, or oil solution.

In some embodiments, the composition further comprises an emollient, a humectant, a thickening agent, a preservative, a penetration enhancer, an anti-oxidant, an odor masking agent, or any combination thereof. In some embodiments, the composition further comprises a preservative. In some embodiments, the preservative is vitamin E.

In some embodiments, the composition further comprises a fatty acid vehicle. In some embodiments, fatty acid vehicle is present in an amount of from about 0, 0.1% to about 99% of the composition. In some embodiments, the fatty acid vehicle is a C14 to C22 fatty acid. In some embodiments, the fatty acid vehicle comprises linoleic acid.

In some embodiments, the composition comprises an oil in an amount of about 0, 0.1% to about 100% (w/w) of the composition. In some embodiments, the oil is derived from a natural source. In some embodiments, the oil is derived from plants, plant seeds, or nuts

In one aspect, provided herein, is a method of treating a disease or disorder of the posterior of the eye in a patient suffering from the disease or disorder comprising administering a therapeutically effective amount of 4,5-dihydro-N-[4-[[4-(1-methylethoxy) phenyl] methyl]phenyl]-1H-imadazol-2-amine or a pharmaceutically acceptable ester or salt thereof to the periorbital skin of an eye of the patient. In some embodiments, the disease or disorder of the posterior of the eye comprises a retinal disease. In some embodiments, the retinal disease comprises hemorrhage from the retinal or choroidal vasculature. In some embodiments, the hemorrhage is caused by systemic hypertension, diabetes, fatty liver disease, obesity, shaken baby syndrome, head trauma, anemia, or leukemia. In some embodiments, the retinal disease or disorder comprises plasma leakage from the retinal or choroidal vasculature. In some embodiments, the plasma leakage is caused by systemic hypertension, diabetes, fatty liver disease, obesity, shaken baby syndrome, head trauma, anemia, or leukemia. In some embodiments, the retinal disease or disorder comprises macular edema formation involving the retinal or choroidal vasculature. In some embodiments, the disease or disorder of the posterior of the eye is age-related macular degeneration (wet and dry forms), dry and wet macular degeneration, lattice Degeneration, macular hole, macular pucker, lattice degeneration, retinal tear, retinal detachment, retinal artery occlusion, retinal vein occlusion, central retinal vein occlusion, intraocular tumors, pediatric, neonatal or inherited retinal disorders, hereditary retinal dystrophies, geographic atrophy, retinitis pigmentosa (including Leber congenital amaurosis), cytomegalovirus (cmv) retinal infection, infectious retinitis, retinoblastoma, endophthalmitis, chorioretinitis, myopic macular degeneration, and normal-tension glaucoma, retinal degeneration in glaucoma, various retinopathies, including but not limited to diabetic retinopathy, retinopathy of prematurity, Sickel cell retinopathy, radiation/solar retinopathy, central serous retinopathy, hypertensive retinopathy, peripheral retinopathy and neuropathy; macular edema, retinal hemorrhage, diabetic macular edema, diabetic macular ischemia, geographic atrophy, Stargardt disease, or refractive errors (myopia, hyperopia, and astigmatism). In some embodiments, the method further comprises administering to the patient an additional therapeutic agent. In some embodiments, the additional therapeutic agent is a VEGF antibody, a small molecule VEGF antagonist, a siRNA targeting a VEGF receptor, a TNFα antibody, a small molecule TNFα receptor antagonist, a siRNA targeting the TNFα receptor, an inflammatory cytokine receptor antagonist, an antibody against an inflammatory cytokine, a tyrosine kinase inhibitor, a serine/threonine-protein kinase inhibitors, a kinase inhibitor, a steroidal anti-inflammatory agent, a non-steroidal anti-inflammatory agent, an immunosuppressant, an anti-cholinergic agent, thalidomide, a prostaglandin receptor antagonist, a neuroprotective agent, a neurotrophic agent, a neuro-regenerative agent, an ocular hypotensive agent, an antibiotics, an antiviral agent, a complement inhibitor, an interleukin receptor inhibitor, a leukotriene receptor inhibitor, an inhibitor of tumorigenesis and development, an angiogenesis inhibitor, or agents with anti-oxidation or anti-microvascular leakage properties. In some embodiments, the 4,5-dihydro-N-[4-[[4-(1-methylethoxy) phenyl] methyl] phenyl]-1H-imadazol-2-amine is administered prophylactically, as an emergency intervention, or as required to achieve the desired remedial effects. In some embodiments, the 4,5-dihydro-N-[4-[[4-(1-methylethoxy) phenyl] methyl]phenyl]-1H-imadazol-2-amine is administered as a composition. In some embodiments, the 4,5-dihydro-N-[4-[[4-(1-methylethoxy) phenyl] methyl] phenyl]-1H-imadazol-2-amine is present in an amount of about 0.0001% to about 10% (w/w) of the composition. In some embodiments, the composition is an aqueous solution, a non-aqueous solution, an oil solution, a gel, a suspension, an emulsion, a lotion, a cream, or an ointment. In some embodiments, the composition is an ointment. In some embodiments, the ointment comprises petrolatum, beeswax, or cocoa butter. In some embodiments, the ointment comprises petrolatum and medium-chain triglycerides. In some embodiments, the medium-chain triglycerides comprise a mixture of C6, C8, C10 and C12 fatty acids. In some embodiments, the medium-chain triglycerides comprise a mixture of caprylic acid and capric acid. In some embodiments, the ointment comprises petrolatum and medium-chain triglyceride in the ratio of about 1:1 (v/v), about 2:1 (v/v), about 3:1 (v/v), about 4:1 (v/v), about 5:1 (v/v), or about 6:1 (v/v). In some embodiments, the ointment comprises petrolatum and medium-chain triglyceride in the ratio of about 4:1 (v/v). In some embodiments, the composition is an aqueous solution. In some embodiments, the aqueous solution comprises a polyoxyl castor oil. In some embodiments, the polyoxyl castor oil is a polyethylene glycol (PEG)-ylated castor oil. In some embodiments, the polyoxyl castor oil is polyoxyl 35 castor oil. In some embodiments, the polyoxyl 35 castor oil is present in an amount of about 0.1% to about 5%, about 0.1% to about 10%, about 0.1% to about 15%, or about 0.1% to about 20% (w/w) of the composition. In some embodiments, the composition comprises an ocular surface lubricating agent. In some embodiments, the 4,5-dihydro-N-[4-[[4-(1-methylethoxy) phenyl] methyl]phenyl]-1H-imadazol-2-amine is applied to the periorbital skin of at least one eye of the patient by dropper, pump, spray, click pen or roller/reservoir device. In some embodiments, the 4,5-dihydro-N-[4-[[4-(1-methylethoxy) phenyl] methyl] phenyl]-1H-imadazol-2-amine is applied to the periorbital skin of at least one eye of the patient by brush, Q-tip, or spatula and where the application process is optionally preceded by using a graduated dropper, syringe, click pen or pipette. In some embodiments, periorbital skin penetration is assisted by tape-stripping, microdermabrasion, solvent, pulsed laser, iontophoresis, or combinations thereof. In some embodiments, the 4,5-dihydro-N-[4-[[4-(1-methylethoxy) phenyl] methyl] phenyl]-1H-imadazol-2-amine is administered to the periorbital skin of each eye of the patient four times per day, three times per day, twice per day, once per day, once every other day, once every three days, once every four days, or once every seven days. In some embodiments, the 4,5-dihydro-N-[4-[[4-(1-methylethoxy) phenyl] methyl] phenyl]-1H-imadazol-2-amine is administered once per day. In some embodiments, the method comprises administering the composition to the periorbital skin above the upper eyelid, below the lower eyelid, or both above the upper and below the lower eyelids. In some embodiments, the additional therapeutic agent is an omega-3 fatty acid or a pharmaceutically acceptable ester or salt thereof. In some embodiments, the 4,5-dihydro-N-[4-[[4-(1-methylethoxy) phenyl] methyl] phenyl]-1H-imadazol-2-amine and the omega-3 fatty acid or a pharmaceutically acceptable ester or salt thereof are formulated and administered as a single composition.

In an additional aspect provided herein is a method of treating a disease or disorder of the posterior of the eye in a patient suffering from the disease or disorder comprising administering a therapeutically effective amount of 4,5-dihydro-N-[4-[[4-(1-methylethoxy) phenyl] methyl]phenyl]-1H-imadazol-2-amine or a pharmaceutically acceptable ester or salt thereof to the ocular surface of an eye of the patient. In some embodiments, the disease or disorder of the posterior of the eye comprises a retinal disease. In some embodiments, the retinal disease comprises hemorrhage from the retinal or choroidal vasculature. In some embodiments, the hemorrhage is caused by systemic hypertension, diabetes, fatty liver disease, obesity, shaken baby syndrome, head trauma, anemia, or leukemia. In some embodiments, the retinal disease or disorder comprises plasma leakage from the retinal or choroidal vasculature. In some embodiments, the plasma leakage is caused by systemic hypertension, diabetes, fatty liver disease, obesity, shaken baby syndrome, head trauma, anemia, or leukemia. In some embodiments, the retinal disease or disorder comprises macular edema formation in the retinal or choroidal vasculature. In some embodiments, the disease or disorder of the posterior of the eye is age-related macular degeneration (wet and dry forms), dry and wet macular degeneration, lattice Degeneration, macular hole, macular pucker, lattice degeneration, retinal tear, retinal detachment, retinal artery occlusion, retinal vein occlusion, central retinal vein occlusion, intraocular tumors, pediatric, neonatal or Inherited retinal disorders, hereditary retinal dystrophies, geographic atrophy, retinitis pigmentosa (including Leber congenital amaurosis), cytomegalovirus (cmv) retinal infection, infectious retinitis, retinoblastoma, endophthalmitis, chorioretinitis, myopic macular degeneration, and normal-tension glaucoma, retinal degeneration in glaucoma; various retinopathies, including but not limited to diabetic retinopathy, retinopathy of prematurity, Sickel cell retinopathy, radiation/solar retinopathy, central serous retinopathy, hypertensive retinopathy, peripheral retinopathy and neuropathy; macular edema, retinal hemorrhage, diabetic macular edema, diabetic macular ischemia, geographic atrophy, Stargardt disease, or refractive errors (myopia, hyperopia, and astigmatism). In some embodiments, the retinal disease or disorder is age-related macular degeneration. In some embodiments, the disease or disorder of the posterior of the eye is posterior uveitis. In some embodiments, the method further comprises administering to the patient an additional therapeutic agent. In some embodiments, the additional therapeutic agent is a small molecule VEGF antagonist, a siRNA targeting A VEGF receptor, a small molecule TNFα receptor antagonist, a siRNA targeting the TNFα receptor, an inflammatory cytokine receptor antagonist, a tyrosine kinase inhibitor, a serine/threonine-protein kinase inhibitors, a kinase inhibitor, a steroidal anti-inflammatory agent, a non-steroidal anti-inflammatory agent, an immunosuppressant, an anti-cholinergic agent, thalidomide, a prostaglandin receptor antagonist, a neuroprotective agent, a neuro-regenerative agent, an ocular hypotensive agent, an antibiotics, an antiviral agent, a complement inhibitor, an interleukin receptor inhibitor, a leukotriene receptor inhibitor, an inhibitor of tumorigenesis and development, an angiogenesis inhibitor, or agents with anti-oxidation or anti-microvascular leakage properties. In some embodiments, the 4,5-dihydro-N-[4-[[4-(1-methylethoxy) phenyl]methyl] phenyl]-1H-imadazol-2-amine is administered as a composition, wherein the composition is an aqueous solution, a non-aqueous solution, an oil solution, a gel, a suspension, an emulsion, a cream, an ointment, in liposomes or in nanoparticles. In some embodiments, the 4,5-dihydro-N-[4-[[4-(1-methylethoxy) phenyl] methyl] phenyl]-1H-imadazol-2-amine is present in an amount of about 0.0001% to about 10% (w/w) of the composition. In some embodiments, the composition is an aqueous solution. In some embodiments, the aqueous solution comprises a polyoxyl castor oil. In some embodiments, the polyoxyl castor oil is a polyethylene glycol (PEG)-ylated castor oil. In some embodiments, the polyoxyl castor oil is polyoxyl 35 castor oil. In some embodiments, the polyoxyl 35 castor oil is present in an amount of about 0.1% to about 5%, about 0.1% to about 10%, about 0.1% to about 15%, or about 0.1% to about 20% (w/w) of the composition. In some embodiments, the composition comprises an ocular surface lubricating agent. In some embodiments, the 4,5-dihydro-N-[4-[[4-(1-methylethoxy) phenyl] methyl]phenyl]-1H-imadazol-2-amine is administered to the ocular surface of each eye of the patient four times per day, three times per day, twice per day, once per day, once every other day, once every three days, once every four days, or once every seven days. In some embodiments, the 4,5-dihydro-N-[4-[[4-(1-methylethoxy) phenyl] methyl] phenyl]-1H-imadazol-2-amine is administered once per day. In some embodiments, the additional therapeutic agent is an omega-3 fatty acid or a pharmaceutically acceptable ester or salt thereof. In some embodiments, the omega-3 fatty acid or a pharmaceutically acceptable ester or salt thereof is administered periorbitally as a composition.

In an additional aspect provided herein is a method of treating uveitis in a patient suffering from uveitis comprising administering to the eye of the patient a therapeutically effective amount of 4,5-dihydro-N-[4-[[4-(1-methylethoxy) phenyl] methyl] phenyl]-1H-imadazol-2-amine or a pharmaceutically acceptable ester or salt thereof. In an additional aspect provided herein is a patient suffering from pterygium comprising administering to the eye of the patient a therapeutically effective amount of 4,5-dihydro-N-[4-[[4-(1-methylethoxy) phenyl]methyl] phenyl]-1H-imadazol-2-amine or a pharmaceutically acceptable ester or salt thereof. In an additional aspect provided herein is a method of treating an ocular disease or disorder in a patient suffering from the disease or disorder, comprising administering to the eye of the patient a therapeutically effective amount of 4,5-dihydro-N-[4-[[4-(1-methylethoxy) phenyl] methyl]phenyl]-1H-imadazol-2-amine or a pharmaceutically acceptable ester or salt thereof, wherein the ocular disease or disorder is anterior segment dysgenesis, cataract, iritis, pterygium, keratoconjunctivitis, keratitis, conjunctivitis, keratoconus, ectatic disorders (including keratoglobus, pellucid marginal degeneration), Pseudophakic and aphakic bullous keratopathy, episcleritis, corneal ulceration, corneal dysplasia, corneal ulceration, Fuchs' endothelial dystrophy and other corneal dystrophies (including lattice, granular, macular, and map-dot fingerprint), ocular cicatricial pemphigoid, Stevens Johnson syndrome, acute and chronic uveitis (anterior uveitis, intermediate uveitis), trauma to the cornea, conjunctiva and anterior segment including iris trauma, or penetrating ocular trauma. In an additional aspect provided herein is a method of treating an ocular disease or disorder affecting the eyelid of a patient suffering from the disease or disorder, comprising administering to the eye of the patient a therapeutically effective amount of 4,5-dihydro-N-[4-[[4-(1-methylethoxy) phenyl] methyl] phenyl]-1H-imadazol-2-amine or a pharmaceutically acceptable ester or salt thereof, wherein the ocular disease or disorder affecting the eyelid is blepharitis, blepharospasm, chalazion, ptosis, coloboma, dermatochalasis, ectropion, entropion, trichiasis, stye, meibomianitis, Meibomian Gland Dysfunction, lacrimal gland obstruction, lacrimal gland obstruction, seborrheic keratitis, actinic keratitis, bacterial infection, or viral infection. In some embodiments, the 4,5-dihydro-N-[4-[[4-(1-methylethoxy) phenyl] methyl] phenyl]-1H-imadazol-2-amine is administered topically to the surface of the eye as a composition. In some embodiments, the 4,5-dihydro-N-[4-[[4-(1-methylethoxy) phenyl] methyl] phenyl]-1H-imadazol-2-amine is administered periorbitally as a composition. In some embodiments, the 4,5-dihydro-N-[4-[[4-(1-methylethoxy) phenyl] methyl]phenyl]-1H-imadazol-2-amine is administered topically to the surface of the eye as a composition and separately applied periorbitally as a composition. In some embodiments, the composition is an aqueous solution, a non-aqueous solution, an oil solution, a gel, a suspension, an emulsion, a cream, or ointment, in liposomes, or in nanoparticles with or without co-incorporation of an siRNA or an antibody. In some embodiments, the composition is an aqueous solution. In some embodiments, the aqueous solution comprises a polyoxyl castor oil. In some embodiments, the polyoxyl castor oil is a polyethylene glycol (PEG)-ylated castor oil. In some embodiments, the polyoxyl castor oil is polyoxyl 35 castor oil. In some embodiments, the polyoxyl 35 castor oil is present in an amount of about 0.1% to about 5%, about 0.1% to about 10%, about 0.1% to about 15%, or about 0.1% to about 20% (w/w) of the composition. In some embodiments, the composition comprises an ocular surface lubricating agent. In some embodiments, the 4,5-dihydro-N-[4-[[4-(1-methylethoxy) phenyl] methyl] phenyl]-1H-imadazol-2-amine is topically applied by dropper, pump, spray, click pen or roller/reservoir device. In some embodiments, the 4,5-dihydro-N-[4-[[4-(1-methylethoxy) phenyl] methyl] phenyl]-1H-imadazol-2-amine is topically applied to the periorbital skin of at least one eye by brush, Q-tip, or spatula and where the application process may be preceded by using a graduated dropper, syringe, click pen or pipette. In some embodiments, the 4,5-dihydro-N-[4-[[4-(1-methylethoxy) phenyl] methyl]phenyl]-1H-imadazol-2-amine is administered to the patient four times per day, three times per day, twice per day, once per day, once every other day, once every three days, once every four days, or once every seven days. In some embodiments, the 4,5-dihydro-N-[4-[[4-(1-methylethoxy) phenyl] methyl] phenyl]-1H-imadazol-2-amine is administered once per day. In some embodiments, the method further comprises administering to the periorbital skin of the eye of the patient a topical pharmaceutical composition comprising an omega-3 fatty acid, or a pharmaceutically acceptable ester or salt thereof.

In an additional aspect provided herein is a pharmaceutical composition suitable for topical periorbital administration, comprising 4,5-dihydro-N-[4-[[4-(1-methylethoxy) phenyl]methyl] phenyl]-1H-imadazol-2-amine or a pharmaceutically acceptable ester or salt thereof. In some embodiments, the composition is an aqueous solution, a non-aqueous solution, an oil solution, a gel, a suspension, an emulsion, a cream, an ointment, in liposomes or in nanoparticles with or without co-incorporation of an siRNA or an antibody. In some embodiments, the composition is formulated as an oil solution. In some embodiments, the composition comprises an oil in an amount of about 1% to about 100% (w/w) of the composition. In some embodiments, the composition comprises an oil in an amount of at least about 90%, at least about 91%, at least about 92%, at least about 93%, at least about 94%, at least about 95%, at least about 96%, at least about 97%, at least about 98%, at least about 99%, at least about 99.5%, at least ab out 99.6%, at least about 99.7%, at least about 99.8%, at least about 99.9%, at least about 99.95%, at least about 99.96%, at least about 99.97%, at least about 99.98%, or at least about 99.99% (w/w) of the composition. In some embodiments, the oil is derived from a natural source. In some embodiments, the oil is derived from plants, plant seeds, or nuts, or any combination thereof. In some embodiments, the oil comprises a medium-chain triglyceride. In some embodiments, the medium-chain triglyceride comprise a mixture of C6, C8, C10 or C12 fatty acids. In some embodiments, the 4,5-dihydro-N-[4-[[4-(1-methylethoxy) phenyl] methyl] phenyl]-1H-imadazol-2-amine is present in an amount of from about 0.00015% to about 10% (w/w) of the composition. In some embodiments, the pharmaceutical composition is configured to dispense from about 0.5 microgram (μg) to about 5 milligrams (mg) of the 4,5-dihydro-N-[4-[[4-(1-methylethoxy) phenyl] methyl] phenyl]-1H-imadazol-2-amine per eye per administration. In some embodiments, the pharmaceutical composition further comprises an emollient, a humectant, a thickening agent, a preservative, a penetration enhancer, or any combination thereof. In some embodiments, the pharmaceutical composition further comprises an omega-3 fatty acid or a pharmaceutically acceptable ester or salt thereof.

In an additional aspect provided herein is a pharmaceutical composition suitable for topical ocular surface administration, comprising 4,5-dihydro-N-[4-[[4-(1-methylethoxy) phenyl]methyl] phenyl]-1H-imadazol-2-amine, or a pharmaceutically acceptable ester or salt thereof, and a polyoxyl castor oil. In some embodiments, the polyoxyl castor oil is a polyethylene glycol (PEG)-ylated castor oil. In some embodiments, the ratio of PEG to castor oil is from about 20:1 to about 50:1. In some embodiments, the polyoxyl castor oil is polyoxyl 35 castor oil. In some embodiments, the polyoxyl 35 castor oil is present in an amount of about 0.1% to about 5%, about 0.1% to about 10%, about 0.1% to about 15%, or about 0.1% to about 20% (w/w) of the composition. In some embodiments, the polyoxyl 35 castor oil is present in an amount of about 1% (w/w) of the composition. In some embodiments, the pharmaceutical composition further comprises an ocular surface lubricating agent. In some embodiments, the ocular surface lubricating agent is polyethylene glycol, propylene glycol, polyvinyl alcohol, castor oil or glycerol. In some embodiments, the ocular surface lubricating agent is present in an amount of about 0.05% to about 2% (w/w) of the composition. In some embodiments, the pharmaceutical composition further comprises a buffer. In some embodiments, the pharmaceutical composition has a pH of from about 6.5 to about 8.5. In some embodiments, the 4,5-dihydro-N-[4-[[4-(1-methylethoxy) phenyl] methyl] phenyl]-1H-imadazol-2-amine is present in an amount of from about 0.0001% to about 10% (w/w) of the composition.

In an additional aspect provided herein is a method of promoting ocular health or preventing or treating ocular disease in a subject, comprising administering to the periorbital skin of an eye the subject a topical pharmaceutical composition comprising an omega-3 fatty acid or a pharmaceutically acceptable ester or salt thereof. In some embodiments, the omega-3 fatty acid is isolated from fish tissue. In some embodiments, the omega-3 fatty acid is isolated from a plant source. In some embodiments, the omega-3 fatty acid comprises alpha-linolenic acid (ALA), eicosapentaenoic acid (EPA), docosahexaenoic acid (DHA), or any combination thereof. In some embodiments, the omega-3 fatty acid comprises DHA. In some embodiments, the omega-3 fatty acid comprises EPA. In some embodiments, the omega-3 fatty acid is administered in an amount of from about 0.1 mg to about 3000 mg, about 0.1 mg to about 1000 mg, about 0.1 mg to about 500 mg, about 0.1 mg to about 200 mg, or about 0.1 mg to about 100 mg. In some embodiments, the topical pharmaceutical composition is formulated as a cream, emulsion, ointment, or oil solution. In some embodiments, the topical pharmaceutical composition further comprises an emollient, a humectant, a thickening agent, a preservative, a penetration enhancer, an anti-oxidant, an odor masking agent, or any combination thereof. In some embodiments, the topical pharmaceutical composition further comprises a preservative. In some embodiments, the topical pharmaceutical composition further comprises 4,5-dihydro-N-[4-[[4-(1-methylethoxy) phenyl]methyl] phenyl]-1H-imadazol-2-amine, or a pharmaceutically acceptable ester or salt thereof. In some embodiments, the topical pharmaceutical composition is administered with a bottle with a roller ball, a click pen brush, a pump bottle, or an eye drop bottle and Q-tip. In some embodiments, promoting ocular health, preventing or treating ocular disease comprises treating or preventing dry eye disease and ocular discomfort, irritation, pain and stress, chemical burns, anterior segment dysgenesis, cataract, iritis, pterygium, keratoconjunctivitis, keratitis, conjunctivitis, keratoconus, ectatic disorders (including keratoglobus, pellucid marginal degeneration), Pseudophakic and aphakic bullous keratopathy, episcleritis, corneal ulceration, corneal dysplasia, corneal ulceration, Fuchs' endothelial dystrophy and other corneal dystrophies (including lattice, granular, macular, and map-dot fingerprint), ocular cicatricial pemphigoid, Stevens Johnson syndrome, acute and chronic uveitis (anterior uveitis, intermediate uveitis), trauma to the cornea, conjunctiva and anterior segment including iris trauma, penetrating ocular trauma, blepharitis, blepharospasm, chalazion, ptosis, coloboma, dermatochalasis, ectropion, entropion, trichiasis, stye, meibomianitis, Meibomian Gland Dysfunction, lacrimal gland obstruction, lacrimal gland obstruction, seborrheic keratitis, actinic keratitis, bacterial infection, or viral infection, age-related macular degeneration (wet and dry forms), dry and wet macular degeneration, lattice Degeneration, macular hole, macular pucker, lattice degeneration, retinal tear, retinal detachment, retinal artery occlusion, retinal vein occlusion, central retinal vein occlusion, intraocular tumors, pediatric, neonatal or Inherited retinal disorders, hereditary retinal dystrophies, geographic atrophy, retinitis pigmentosa (including Leber congenital amaurosis), cytomegalovirus (cmv) retinal infection, infectious retinitis, retinoblastoma, endophthalmitis, chorioretinitis, myopic macular degeneration, and normal-tension glaucoma, retinal degeneration in glaucoma; various retinopathies, including but not limited to diabetic retinopathy, retinopathy of prematurity, Sickel cell retinopathy, radiation/solar retinopathy, central serous retinopathy, hypertensive retinopathy, peripheral retinopathy and neuropathy; macular edema, retinal hemorrhage, diabetic macular edema, diabetic macularischemia, geographic atrophy, Stargardt disease, refractive errors (myopia, hyperopia, and astigmatism), lymphatic malformations of the orbit (a.k.a. orbital lymphangiomas), thyroid eye disease (Graves' Eye Disease), or acute and chronic uveitis (including intermediate uveitis, posterior uveitis, panuveitis). In some embodiments, promoting ocular health, preventing or treating ocular disease comprises treating or preventing dry eye. In some embodiments, promoting ocular health, preventing or treating ocular disease comprises treating or preventing wet or dry age-related macular degeneration. In some embodiments, promoting ocular health, preventing or treating ocular disease comprises treating or preventing various retinopathies, including but not limited to diabetic retinopathy, retinopathy of prematurity. In some embodiments, the topical pharmaceutical composition is administered to the patient four times per day, three times per day, twice per day, once per day, once every other day, once every three days, once every four days, or once every seven days. In some embodiments, the method comprises administering the composition to the periorbital skin above the upper eyelid, below the lower eyelid, or both above the upper and below the lower eyelids. In some embodiments, administering the composition to the periorbital skin results in a tissue concentration of the omega-3 fatty acid of at least 110 micrograms/gram in the retina of the eye of the subject 30 minutes after administration greater than compared to baseline.

In an additional aspect provided herein is a pharmaceutical composition suitable for topical periorbital administration, comprising an omega-3 fatty acid, or a pharmaceutically acceptable ester or salt thereof, and a pharmaceutically acceptable excipient. In some embodiments, the omega-3 fatty acid is isolated from fish tissue. In some embodiments, the omega-3 fatty acid is isolated from a plant source. In some embodiments, the omega-3 fatty acid comprises alpha-linolenic acid (ALA), eicosapentaenoic acid (EPA), docosahexaenoic acid (DHA), or any combination thereof. In some embodiments, the omega-3 fatty acid comprises DHA. In some embodiments, the omega-3 fatty acid comprises EPA. In some embodiments, the omega-3 fatty acid is present in an amount of about 0.01% to about 100% (w/w) of the composition. In some embodiments, the composition is formulated as a cream, emulsion, ointment, or oil solution. In some embodiments, the composition further comprises an emollient, a humectant, a thickening agent, a preservative, a penetration enhancer, an anti-oxidant, an odor masking agent, or any combination thereof. In some embodiments, the formulation further comprises a preservative. In some embodiments, the preservative is vitamin E. In some embodiments, the formulation further comprises a fatty acid vehicle. In some embodiments, the fatty acid vehicle is present in an amount of from about 0.1% to about 99% of the composition. In some embodiments, the fatty acid vehicle is a C14 to C22 fatty acid. In some embodiments, the fatty acid vehicle comprises linoleic acid. In some embodiments, the formulation further comprises an oil in an amount of about 1% to about 100% (w/w) of the composition. In some embodiments, the oil is derived from a natural source. In some embodiments, the oil is derived from plants, plant seeds, or nuts. In some embodiments, the formulation further comprises 4,5-dihydro-N-[4-[[4-(1-methylethoxy) phenyl] methyl] phenyl]-1H-imadazol-2-amine, or a pharmaceutically acceptable ester or salt thereof.

In an additional aspect provided herein is a method of treating a disease or disorder of the posterior of the eye in a patient suffering from the disease or disorder comprising administering a therapeutically effective amount of 4,5-dihydro-N-[4-[[4-(1-methylethoxy) phenyl] methyl]phenyl]-1H-imadazol-2-amine, or a pharmaceutically acceptable ester or salt thereof, to the exterior skin of the eyelid of an eye of the patient. In some embodiments, the disease or disorder of the posterior of the eye comprises a retinal disease. In some embodiments, the retinal disease comprises hemorrhage from the retinal or choroidal vasculature. In some embodiments, the hemorrhage is caused by systemic hypertension, diabetes, fatty liver disease, obesity, shaken baby syndrome, head trauma, anemia, or leukemia. In some embodiments, the retinal disease or disorder comprises plasma leakage from the retinal or choroidal vasculature. In some embodiments, the plasma leakage is caused by systemic hypertension, diabetes, fatty liver disease, obesity, shaken baby syndrome, head trauma, anemia, or leukemia. In some embodiments, the retinal disease or disorder comprises macular edema formation involving the retinal or choroidal vasculature. In some embodiments, the disease or disorder of the posterior of the eye is age-related macular degeneration (wet and dry forms), dry and wet macular degeneration, lattice Degeneration, macular hole, macular pucker, lattice degeneration, retinal tear, retinal detachment, retinal artery occlusion, retinal vein occlusion, central retinal vein occlusion, intraocular tumors, pediatric, neonatal or Inherited retinal disorders, hereditary retinal dystrophies, geographic atrophy, retinitis pigmentosa (including Leber congenital amaurosis), cytomegalovirus (cmv) retinal infection, infectious retinitis, retinoblastoma, endophthalmitis, chorioretinitis, myopic macular degeneration, and normal-tension glaucoma, retinal degeneration in glaucoma; various retinopathies, including but not limited to diabetic retinopathy, retinopathy of prematurity, Sickel cell retinopathy, radiation/solar retinopathy, central serous retinopathy, hypertensive retinopathy, peripheral retinopathy and neuropathy; macular edema, retinal hemorrhage, diabetic macular edema, diabetic macularischemia, geographic atrophy, Stargardt disease, or refractive errors (myopia, hyperopia, and astigmatism). In some embodiments, the retinal disease or disorder is age-related macular degeneration. In some embodiments, the disease or disorder of the posterior of the eye is posterioruveitis. In some embodiments, the method further comprises administering to the patient an additional therapeutic agent. In some embodiments, the additional therapeutic agent is a VEGF antibody, a small molecule VEGF antagonist, a siRNA targeting a VEGF receptor, a TNFα antibody, a small molecule TNFα receptor antagonist, a siRNA targeting the TNFα receptor, an inflammatory cytokine receptor antagonist, an antibody against an inflammatory cytokine, a tyrosine kinase inhibitor, a serine/threonine-protein kinase inhibitors, a kinase inhibitor, a steroidal anti-inflammatory agent, a non-steroidal anti-inflammatory agent, an immunosuppressant, an anti-cholinergic agent, thalidomide, a prostaglandin receptor antagonist, a neuroprotective agent, a neurotrophic agent, a neuro-regenerative agent, an ocular hypotensive agent, an antibiotics, an antiviral agent, a complement inhibitor, an interleukin receptor inhibitor, a leukotriene receptor inhibitor, an inhibitor of tumorigenesis and development, an angiogenesis inhibitor, or agents with anti-oxidation or anti-microvascular leakage properties. In some embodiments, the 4,5-dihydro-N-[4-[[4-(1-methylethoxy) phenyl] methyl] phenyl]-1H-imadazol-2-amine is administered prophylactically, as an emergency intervention, or as required to achieve the desired remedial effects. In some embodiments, the 4,5-dihydro-N-[4-[[4-(1-methylethoxy) phenyl] methyl]phenyl]-1H-imadazol-2-amine is administered as a composition. In some embodiments, the 4,5-dihydro-N-[4-[[4-(1-methylethoxy) phenyl] methyl] phenyl]-1H-imadazol-2-amine is present in an amount of about 0.0001% to about 10% (w/w) of the composition. In some embodiments, the composition is an aqueous solution, a non-aqueous solution, an oil solution, a gel, a suspension, an emulsion, a lotion, a cream, or an ointment. In some embodiments, the composition is an ointment. In some embodiments, the ointment comprises petrolatum, beeswax, or cocoa butter. In some embodiments, ointment comprises petrolatum and medium-chain triglycerides. In some embodiments, the medium-chain triglycerides comprise a mixture of C6, C8, C10 and C12 fatty acids. In some embodiments, the medium-chain triglycerides comprise a mixture of caprylic acid and capric acid. In some embodiments, the ointment comprises petrolatum and medium-chain triglyceride in the ratio of about 1:1 (v/v), about 2:1 (v/v), about 3:1 (v/v), about 4:1 (v/v), about 5:1 (v/v), or about 6:1 (v/v). In some embodiments, the ointment comprises petrolatum and medium-chain triglyceride in the ratio of about 4:1 (v/v). In some embodiments, the composition is an aqueous solution. In some embodiments, the aqueous solution comprises a polyoxyl castor oil. In some embodiments, the polyoxyl castor oil is a polyethylene glycol (PEG)-ylated castor oil. In some embodiments, the polyoxyl castor oil is polyoxyl 35 castor oil. In some embodiments, the polyoxyl 35 castor oil is present in an amount of about 0.1% to about 5%, about 0.1% to about 10%, about 0.1% to about 15%, or about 0.1% to about 20% (w/w) of the composition. In some embodiments, the composition comprises an ocular surface lubricating agent. In some embodiments, the 4,5-dihydro-N-[4-[[4-(1-methylethoxy) phenyl] methyl]phenyl]-1H-imadazol-2-amine is applied to the exterior skin of the eyelid of an eye of the patient by dropper, pump, spray, click pen or roller/reservoir device. In some embodiments, the 4,5-dihydro-N-[4-[[4-(1-methylethoxy) phenyl] methyl] phenyl]-1H-imadazol-2-amine is applied to the exterior skin of the eyelid of an eye of the patient by brush, Q-tip, or spatula and where the application process is optionally preceded by using a graduated dropper, syringe, click pen or pipette. In some embodiments, eyelid skin penetration is assisted by tape-stripping, microdermabrasion, solvent, pulsed laser, iontophoresis, or combinations thereof. In some embodiments, the 4,5-dihydro-N-[4-[[4-(1-methylethoxy) phenyl] methyl] phenyl]-1H-imadazol-2-amine is administered to the eyelid skin of each eye of the patient four times per day, three times per day, twice per day, once per day, once every other day, once every three days, once every four days, or once every seven days. In some embodiments, the 4,5-dihydro-N-[4-[[4-(1-methylethoxy) phenyl] methyl] phenyl]-1H-imadazol-2-amine is administered once per day. In some embodiments, the additional therapeutic agent is an omega-3 fatty acid or a pharmaceutically acceptable ester or salt thereof. In some embodiments, the 4,5-dihydro-N-[4-[[4-(1-methylethoxy) phenyl] methyl] phenyl]-1H-imadazol-2-amine and the omega-3 fatty acid or a pharmaceutically acceptable ester or salt thereof are formulated and administered as a single composition.

In an additional aspect provided herein is an active ingredient formulated for topical administration to the periorbital skin of a patient, for use in the manufacture of a medicament for treating a disease or disorder of the posterior of the eye, wherein the formulation delivers a therapeutically effective amount of said active ingredient formulated for topical administration to the periorbital skin of a patient to the posterior of the eye. In some embodiments, the active ingredient has a molecular weight of less than or equal to 1000 Da. In some embodiments, the active ingredient has a molecular weight of 200-500 Da. In some embodiments, 1 milliliter to 10 milliliters of formulation are topically applied to the periorbital skin of one eye of a patient per use, wherein the active ingredient is topically applied using an eye pad. In some embodiments, 3 microliters to 100 microliters of formulation are topically applied directly to the periorbital skin of one eye of a patient per use. In some embodiments, 0.01 milligrams to 10 grams of active ingredient are topically applied to the periorbital skin of one eye of a patient per use, wherein the active ingredient is topically applied using an eye pad. In some embodiments, 0.01 milligrams to 100 milligrams of active ingredient are topically applied directly to the periorbital skin of one eye of a patient peruse. In some embodiments, the formulation further comprises an oil in an amount of about 1% to about 100% (w/w) of the composition. In some embodiments, the oil is derived from a natural source. In some embodiments, the oil is derived from plants, plant seeds, or nuts. In some embodiments, the oil comprises a medium-chain triglyceride. In some embodiments, the oil comprises soybean oil. In some embodiments, the active ingredient is an omega-3 fatty acid or a pharmaceutically acceptable ester or salt thereof. In some embodiments, the omega-3 fatty acid or pharmaceutically acceptable ester or salt thereof comprises alpha-linolenic acid (ALA), eicosapentaenoic acid (EPA), docosahexaenoic acid (DHA), or any combination thereof. In some embodiments, the omega-3 fatty acid or pharmaceutically acceptable ester or salt thereof comprises DHA. In some embodiments, the omega-3 fatty acid or pharmaceutically acceptable ester or salt thereof comprises EPA. In some embodiments, the omega-3 fatty acid or pharmaceutically acceptable ester or salt thereof is present in an amount of about 0.01% to about 100% (w/w) of the composition. In some embodiments, administering 6.7 mg of the formulation results in a tissue concentration of the omega-3 fatty acid 30 in the posterior of the eye of the patient 30 minutes after administration of about 110 micrograms/gram greater than compared to baseline. In some embodiments, the formulation further comprises 4,5-dihydro-N-[4-[[4-(1-methylethoxy) phenyl] methyl] phenyl]-1H-imadazol-2-amine or a pharmaceutically acceptable ester or salt thereof. In some embodiments, the expected range of active ingredient detectable in the posterior tissue of the eye is about 0.1 μg to about 1600 μg per gram of posterior tissue. In some embodiments, the active ingredient is administered to the periorbital skin of each eye of the patient four times per day, three times per day, twice per day, or once per day. In some embodiments, the active ingredient is 4,5-dihydro-N-[4-[[4-(1-methylethoxy) phenyl] methyl] phenyl]-1H-imadazol-2-amine or a pharmaceutically acceptable ester or salt thereof. In some embodiments, the 4,5-dihydro-N-[4-[[4-(1-methylethoxy) phenyl] methyl] phenyl]-1H-imadazol-2-amine is present in an amount of from about 0.00005% to about 10% (w/w) of the composition. In some embodiments, the formulation further comprises an omega-3 fatty acid or pharmaceutically acceptable ester or salt thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE FIGURES

FIG. 1 shows the anatomy of the periorbital region of the eye.

FIG. 2A shows bioavailability of JV-DE1 in the right eye of rabbits dosed bilaterally with JV-DE1 as a topical ophthalmic formulation directly to the ocular surface.

FIG. 2B shows bioavailability of JV-DE1 in the left eye of rabbits dosed bilaterally with JV-DE1 as a topical ophthalmic formulation directly to the ocular surface.

FIG. 3A shows bioavailability of JV-DE1 in the right eye of rabbits dosed in the right eye with JV-DE1 as a topical ophthalmic formulation directly to the ocular surface.

FIG. 3B shows bioavailability of JV-DE1 in the left eye of rabbits dosed in the right eye with JV-DE1 as a topical ophthalmic formulation directly to the ocular surface.

FIG. 4 illustrates the delivery of compounds (e.g. the compounds disclosed herein) via the cornea route to the anterior segments of the eye, and via the sclera pathway to the posterior segments of the eye, through non-invasive periorbital application.

### DETAILED DESCRIPTION OF THE INVENTION

Provided herein are uses of the compound 4,5-dihydro-N-[4-[[4-(1-methylethoxy) phenyl] methyl] phenyl]-1H-imadazol-2-amine (a.k.a JV-DE1, JV-MD2) as a treatment of ocular diseases. In some embodiments, the ocular diseases are diseases and disorders associated with the posterior segment of the eye. The formulated compound achieves bioavailability in the posterior segment of the eye following application by eye-drop to ocular surface but more favorably by application to the periorbital skin. In some embodiments, the formulated compound is administered through a non-invasive ocular delivery platform (NIODP). A non-invasive ocular delivery platform (NIODP) is a combination of periorbital skin transdermal administration with appropriate drug formulation to deliver ocular drugs, particularly retinal drugs, at above μg/g of ocular tissue.

Embodiments disclosed are directed towards the treatment of ocular diseases, including diseases of the anterior segment, and, unexpectedly, diseases of the posterior segment of the eye. These diseases may arise from the posterior segment of the eye, the anterior segment of the eye, or as a result of systemic diseases such as diabetes, rheumatoid arthritis and systemic hypertension as non-limiting examples. Formulations capable of delivering therapeutically adequate amounts of the compound to the posterior pole of the eye are included, achieved by application to the ocular surface by eye-drops or to the periorbital skin that surrounds the globe.

Also provided herein are methods and compositions for delivery of fatty acids, including omega-3 fatty acids, directed to the eye via periorbital administration. Surprisingly, it has been found herein that omega-3 fatty acids, including docosahexaenoic acid (DHA), can be effectively delivered to and throughout the eye, including to the posterior of the eye, via periorbital administration. Such a route of administration provides several advantages for DHA and other fatty acids that have an oily character making administration by eye drops undesirable. Administration of omega-3 fatty acids in this manner is useful for promoting ocular health, preventing or treat ocular diseases, including age-related macular degeneration (AMD).

Further disclosed herein, are methods and compositions for delivery of fatty acids, including omega-3 fatty acids, directly to the eye via administration to the exterior surface of the eyelid.

### Diseases of the Posterior of the Eye

Retinal diseases compromise vision and the resultant impairment and can eventually lead to total vision loss. They are common and the major risk factors are the “usual suspects”: age, obesity, and smoking. Abnormalities of the retinal and choroidal vasculature, notably hemorrhage and plasma exudation, occur as a result of systemic maladies that include diabetes, high blood pressure, fatty liver disease, obesity, head trauma, anemia, and leukemia.

The retina has two blood supplies, the choroidal and retinal vasculatures. The choroidal circulation supplies blood-based nutrition to the retinal pigmented epithelium, the photoreceptors, and the outer plexiform layer. The retinal circulation supplies blood-borne nutrition to the inner nuclear, plexiform, ganglion, and nerve fiber layers. The macula is supplied by the superior and temporal branches of the central retinal artery. The retinal circulation is anatomically located as to be the source of hemorrhage into the vitreous.

Diseases that originate in the retina per se include but not limited to age-related macular degeneration, macular hole, macular pucker, degenerative, lattice degeneration, retinal tear, retinal detachment, retinal artery occlusion, branch retinal vein occlusion, central retinal vein occlusion, intraocular tumors, pediatric and neonatal retinal disorders, cytomegalovirus (cmv) retinal infection, uveitis, infectious retinitis, central serous retinopathy, retinoblastoma, endophthalmitis and geographic atrophy, retinitis pigmentosa, etc. Retinal neovascularization may also occur as a result of childhood myopia (Bremond-Gignac D, 2020, Med Sci, 36: 763-763). Thus, abnormalities of the vasculature are the dominant feature of many retinal diseases; these include hemorrhage, edema, angiogenesis, inflammation, and fibrosis.

Hemorrhage from the choroidal and/or retinal vasculature is a serious matter. There are currently no drugs “listed” for the treatment of retinal hemorrhage. Anti-VEGF antibodies, very effective in preventing angiogenesis, are of little value in treating vitreous hemorrhage (El Annan, Carvounis P E, 2014, Int Ophthalmol Clin 54: 141-153). It is well known that the release of IP receptor agonist prostacyclin (PGI2) can stimulate vasodilatation and increase blood flow in the ocular microvasculature (Mori A et al., 2007, Eur J Pharmacol 570: 135-141). The use of an IP receptor antagonist during choroidal, or retinal, hemorrhage would produce pronounced vasoconstriction and reduce retinal hemorrhage. Platelet activating factor (PAF) is not only a mediator of ocular inflammation, but can also stimulate corneal and retinal neovascularization. Various PAF antagonists have been shown to decrease microvasculature leakage in anterior uveitis (Lin N and Bazan H, et al. 1991; Rubin R M, Samples J R, et al. 1988), decrease neovascularization in corneal grafts (Cohen R A, Gebhardt B M, et al. 1992), as well as reduce Choroidal Neovascularization in rat model (Zhang H, Yang Y, et al., 2013). The PAF antagonist property of 4,5-dihydro-N-[4-[[4-(1-methylethoxy) phenyl] methyl] phenyl]-1H-imadazol-2-amine would further reduce retinal inflammation and hemorrhage.

Elevated prostacyclin levels have been reported in diabetic retinopathy (Davis T M E, et al. (1981) Br Med J 282: 1259-1262; Lane L S, et al. (1982) Ophthalmol 89: 763-766). Diabetic retinopathy results from lesions in the retinal microcirculation, the choroidal microvasculature may also be involved. It is the most common cause of vitreous hemorrhage. The leakage of blood into the vitreous humor may be visualized as floaters, cobwebs or shadows in mild cases but severe cases result in haziness, streaks, or even a complete loss of vision. The most common cause of vitreous hemorrhage is advanced diabetic retinopathy but it may occur in other retinal diseases such as age-related macular degeneration, retinal vein occlusion, micro-aneurisms, Terson syndrome, proliferative sickle cell retinopathy, and retinal or vitreous detachment.

Angiogenesis is also a prominent feature in the development of certain retinopathies. Vascular endothelial growth factor (VEGF) in some cases produces choroidal neovascularization and anti-VEGF antibodies are widely used as an effective treatment for choroidal neovascularization. A receptor selective IP receptor antagonist, administered systemically, was also shown effective in preventing choroidal neovascularization; this was first disclosed in U.S. Pat. Nos. 9,295,665 and 9,321,745. Such newly formed blood vessels allow tissue blood perfusion but are inclined to hemorrhage and allow plasma exudation, which results in tissue swelling (edema) and a resultant change in retinal dimensions critical for optimal vision. In addition to VEGF, angiogenic factors such as bFGF (D'Amore P., 1994, Invest Ophthalmol Vis Sci 35: 3974-3979) PDGF, SDF-1, HIF-1 (Campochiaro P A., 2015, ProgRetin Eye Res 49: 67-81), PDGF, PIGF (Noma H et al., 2020, J Clin Med 9: 3457), and IGF-1 (Lin S et al, 2017, Cell Prolif. 50:e12390) have been implicated in the pathogenesis of neovascularization. IGF-1 promotes angiogenesis with activation of PI3K/Akt signal pathway (Lin S et al, 2017, Cell Prolif. 50:e12390). The PI3K/AKT/mTOR signal transduction pathway is abnormally activated in many tumorigenesis processes and has a key role in tumorigenesis and development. (Feng Z, Levine A J, Trends Cell Biol. 2010 July; 20(7): 427-434).

A further major contributor to vision loss in retinal disease is macular edema. The macula is essential for central vision. Fluid extravasation into the macular region results in compromised vision due to thickening and distortion of the macula. It has long been known that increased blood flow increases fluid exudation into tissues, notably prostanoid mediated vasodilatation (Williams T J, Peck M J (1977) Nature 270: 530-532). It follows that a reduction in prostacyclin mediated tissue blood flow by an IP receptor antagonist would ameliorate macula edema. PAF can similarly induce vasodilatation and increased plasma exudation into tissue and could, therefore, contribute to macula edema in retinal degenerative and inflammatory diseases. In addition to VEGF and PDGF, MCP-1, ICAM-1, IP-10, PTX-3, IL-6, and IL-8 have also been implicated in the development of macular edema (Noma et al., 2020).

The retinal diseases are the major cause of vision loss. They are characterized by a number of pathologies that include hemorrhage, edema, angiogenesis, inflammation, fibrosis, and atrophy, which may occur as a single event or in permutations. Angiogenesis and resultant sequelae may be abrogated by antibodies directed to Vascular Endothelial Growth Factor (VEGF). Retinal antibody therapy requires injection into the vitreous humor located in the posterior chamber of the globe. Intravitreal injection is essentially undesirable. Much effort continues to be expended in reducing the frequency of antibody injections into the eye by ophthalmologists. Self-application of eye-drops to the ocular surface is typical and preferable for treating eye diseases. However, drugs applied to the ocular surface do not adequately achieve the posterior segment of the eye (Awwad S et al., 2017, Br J Pharmacol 174: 4205-4223; Del Amo E M et al., 2017, Prog Retin Eye Res 57: 134-185). No solid success has been shown (del Amo et al., 2017). The delivery of drugs to the posterior segment of the eye by topical administration has been described as the “holy grail” (Rodrigues G A et al., 2018, Pharm Res 35: 245). Thus, a drug that convincingly reaches the posterior segment tissues and vitreous humor following topical application to the ocular surface would be a breakthrough.

IP receptor antagonism represents an alternative and additional mechanism of action for treating hemorrhage, plasma exudation, neovascularization, and macular edema associated with the retinal and choroidal vasculatures that provide blood-borne nutrition to the tissues that are located in the ocular posterior segment. The current mainstay of therapeutic intervention is intravitreal injection of proteins that sequester VEGF or its receptors. VEGF antibody injection into the vitreous is not without risks. Retinal antibody therapy requires injection into the ocular posterior chamber, which is essentially undesirable. Incidences of endophthalmitis, retinal detachment, and traumatic cataract per injection were reported as 0.16%, 0.17%, and 0.07%, respectively (Michels S, 2006, Br J Ophthalmol 90: 1333-1334). There is added risk with each injection, with patients receiving from 4-12 injections per annum. There is a widely recognized desire to reduce, or even abolish, the number of required intravitreal injections. IP receptor antagonists prevent the release of VEGF, however and according to this present invention, 4,5-dihydro-N-[4-[[4-(1-methylethoxy) phenyl] methyl] phenyl]-1H-imadazol-2-amine may achieve its retinal target without the need of injection.

Independent of VEGF release, IP antagonists would reduce blood perfusion of the microvasculature and thereby reduce edema formation and the potential for hemorrhage. 4,5-dihydro-N-[4-[[4-(1-methylethoxy) phenyl] methyl] phenyl]-1H-imadazol-2-amine (also assigned the numerical identities RO-1138452 and JV-DE1) is unique in that it also possesses PAF receptor antagonist properties, which would inhibit neovascularization and fibrosis. Since blindness is the inevitable outcome for many patients, the addition of 4,5-dihydro-N-[4-[[4-(1-methylethoxy) phenyl] methyl] phenyl]-1H-imadazol-2-amine to the ophthalmologists currently small armamentarium of effective drugs for retinal diseases would be invaluable.

Provided herein is a method of treating a posterior ocular disease, a retinal disease or disorder in a subject comprising administering to the subject a therapeutically effective amount of 4,5-dihydro-N-[4-[[4-(1-methylethoxy) phenyl] methyl] phenyl]-1H-imadazol-2-amine. In some embodiments, the 4,5-dihydro-N-[4-[[4-(1-methylethoxy) phenyl] methyl]phenyl]-1H-imadazol-2-amine is administered to the periorbital skin. In some embodiments, the 4,5-dihydro-N-[4-[[4-(1-methylethoxy) phenyl] methyl] phenyl]-1H-imadazol-2-amine is administered topically to the eye. In some embodiments, the 4,5-dihydro-N-[4-[[4-(1-methylethoxy) phenyl] methyl] phenyl]-1H-imadazol-2-amine is administered topically to the surface of the eye.

In some embodiments, the posterior ocular disease, retinal disease or disorder is age-related macular degeneration (wet and dry forms), dry and wet macular degeneration, lattice Degeneration, macular hole, macular pucker, lattice degeneration, retinal tear, retinal detachment, retinal artery occlusion, retinal vein occlusion, central retinal vein occlusion, intraocular tumors, pediatric, neonatal or Inherited retinal disorders, hereditary retinal dystrophies, geographic atrophy, retinitis pigmentosa (including Leber congenital amaurosis), cytomegalovirus (cmv) retinal infection, infectious retinitis, retinoblastoma, endophthalmitis, chorioretinitis, myopic macular degeneration, and normal-tension glaucoma, retinal degeneration in glaucoma; various retinopathies, including but not limited to diabetic retinopathy, retinopathy of prematurity, Sickel cell retinopathy, radiation/solar retinopathy, central serous retinopathy, hypertensive retinopathy, peripheral retinopathy and neuropathy; macular edema, retinal hemorrhage, diabetic macular edema, diabetic macular ischemia, geographic atrophy, Stargardt disease, or refractive errors (myopia, hyperopia, and astigmatism).

In some embodiments, the retinal disease or disorder is age-related macular degeneration (wet and dry forms). In some embodiments, the retinal disease or disorder is dry age-related macular degeneration. In some embodiments, the retinal disease or disorder is wet age-related macular degeneration.

In some embodiments, the retinal disease or disorder is not retinal neovascularization. In some embodiments, the retinal disease or disorder is not neovascularization. In some embodiments, the retinal disease or disorder does not comprise retinal neovascularization.

### Uveitis

Also provided herein is a method of treating uveitis, the method comprising administering 4,5-dihydro-N-[4-[[4-(1-methylethoxy) phenyl] methyl] phenyl]-1H-imadazol-2-amine to the eye of patient. In some embodiments, the 4,5-dihydro-N-[4-[[4-(1-methylethoxy) phenyl] methyl] phenyl]-1H-imadazol-2-amine is administered periorbitally. In some embodiments, the 4,5-dihydro-N-[4-[[4-(1-methylethoxy) phenyl] methyl] phenyl]-1H-imadazol-2-amine is administered topically to the surface of the eye. In some embodiments, the 4,5-dihydro-N-[4-[[4-(1-methylethoxy) phenyl] methyl] phenyl]-1H-imadazol-2-amine is administered both periorbitally and topically to the surface of the eye.

Uveitis is a condition characterized by inflammation of the part of the eye known as the uvea, which consists of three parts: the iris, the ciliary body, and the choroid. There are 4 types of uveitis. (1) Anterior uveitis, the most common and usually less serious type, affects the iris at the front of the eye; (2) Intermediate uveitis affects the ciliary body and the vitreous; (3) Posterior uveitis affects the retina and the choroid at the back of the eye; (4) Panuveitis affects all parts of the uvea, from the front to the back of the eye. Panuveitis is the most severe form of uveitis, which can also affect the lens, retina, optic nerve, and vitreous, causing reduced vision or blindness. 4,5-dihydro-N-[4-[[4-(1-methylethoxy) phenyl] methyl] phenyl]-1H-imadazol-2-amine may be administered to treat anterior uveitis, intermediate uveitis, posterior uveitis, or panuveitis, or a combination thereof. Uveitis can have infectious or noninfectious causes. Infectious causes include bacterial, fungal, parasitic, and viral infections. Noninfectious causes include immunologic problems, allergies, malignancies, and unknown causes. As the treatment of choice for most types of uveitis, including panuveitis, corticosteroid eye drops have been used to reduce inflammation and pain, oral medications or injections have been used to treat severe cases. Infectious uveitis can be treated by antibiotics, antiparasitics or antivirals, specific to the infectious agent, with or without corticosteroids. For chronic, recurrent, severe uveitis, such as intermediate uveitis, posterior uveitis, and/or panuveitis, that cannot be controlled with short term use of corticosteroids, or in cases of active inflammation that interferes with daily activities, immunomodulating drugs (biologics) delivered by ocular injection are necessary. Thus, methods for the delivery of compounds which treat the disease to the back of the eye in a non-invasive manner provide a substantial improvement over existing treatments.

### Myopia

The treatment of myopia would also benefit from the application of therapeutic interventions to the periorbital skin, which directly overlies the sclera. A widely held concept involves the form deprivation created by an out-of-focus image results in the retina transmitting signals to the sclera and an extension of the long axis of the globe is thereby created by alterations to the sclera (Wang et al., 1997; Gallego et al., 2012). Drugs delivered via the periorbital skin route may result in therapeutically effective drug levels at both the sclera and retina. Interestingly the anti-glaucoma agents brimonidine (Carr et al., 2019) and latanoprost (El-Nimri and Wildsoet, 2018) have been reported to inhibit myopia progression in animal models thereof.

### Diseases of the Anterior of the Eye

Also provided herein are methods of treating certain diseases and disorders associated with the anterior portion of the eye. Examples of such diseases include blepharitis, blepharospasm, chalazion, ptosis, coloboma, dermatochalasis, ectropion, entropion, trichiasis, stye, meibomianitis, Meibomian Gland Dysfunction, lacrimal gland obstruction, lacrimal gland obstruction, seborrheic keratitis, actinic keratitis, bacterial infection, or viral infection, anterior segment dysgenesis, cataract, iritis, pterygium, keratoconjunctivitis, keratitis, conjunctivitis, keratoconus, ectatic disorders (including keratoglobus, pellucid marginal degeneration), Pseudophakic and aphakic bullous keratopathy, episcleritis, corneal ulceration, corneal dysplasia, corneal ulceration, Fuchs' endothelial dystrophy and other corneal dystrophies (including lattice, granular, macular, and map-dot fingerprint), ocular cicatricial pemphigoid, Stevens Johnson syndrome, acute and chronic uveitis (anterior uveitis, intermediate uveitis), trauma to the cornea, conjunctiva and anterior segment including iris trauma, penetrating ocular trauma. In some embodiments, the disease or disorders comprise anterior ocular inflammatory diseases. In some embodiments, the disease or disorder is an anterior ocular disorder associated with inflammation, hemorrhage, edema, or fibrosis. In some embodiments, these diseases or disorders can be treated with a prostanoid IP receptor antagonist or inhibitor, such as 4,5-dihydro-N-[4-[[4-(1-methylethoxy) phenyl] methyl] phenyl]-1H-imadazol-2-amine (a.k.a. JV-DE1). In some embodiments the 4,5-dihydro-N-[4-[[4-(1-methylethoxy) phenyl] methyl]phenyl]-1H-imadazol-2-amine may treat diseases of the anterior of the eye through administration to the surface of the eye (e.g., in a droplet formulation). In some embodiments, the 4,5-dihydro-N-[4-[[4-(1-methylethoxy) phenyl] methyl] phenyl]-1H-imadazol-2-amine may treat diseases of the anterior of the eye through administration through the periorbital skin. In some embodiments, the 4,5-dihydro-N-[4-[[4-(1-methylethoxy) phenyl] methyl] phenyl]-1H-imadazol-2-amine may treat diseases of the anterior of the eye through administration to the surface of the eye (e.g., in a droplet formulation) and administration to the periorbital skin. In some embodiments, these diseases or disorders can be treated with an omega-3 fatty acid or a pharmaceutically acceptable ester or salt thereof. In some embodiments, the omega-3 fatty acid or a pharmaceutically acceptable ester or salt thereof is administered periorbitally to a patient to treat a disease or disorder of the anterior segment of the eye of the patient.

Inflammation plays an important role in the development of certain anterior ocular disease, including dry eye, uveitis, and pterygium. It has been proposed that VEGF is a core molecule in the cross talk between inflammation and these diseases, which may explain the high incidence of coexistence of these diseases (Liu C, Song Y, et al. 2020).

IP receptor stimulation potently and highly efficaciously promotes VEGF release from human immune cells when macrophages were primed with TNFα to mimic an inflammatory event. The IP antagonist 4,5-dihydro-N-[4-[[4-(1-methylethoxy) phenyl] methyl]phenyl]-1H-imadazol-2-amine abolished the VEGF release induced by IP receptor agonist cicaprostin the presence of TNFα (U.S. Pat. No. 9,321,745). Thus, 4,5-dihydro-N-[4-[[4-(1-methylethoxy) phenyl] methyl] phenyl]-1H-imadazol-2-amine may provide a therapeutic effect in anterior ocular diseases linked with inflammation, due to its anti-inflammatory effects.

Platelet activating factor (PAF) is a mediator of inflammation and can cause injury to the eye. Various PAF antagonists, such as 4,5-dihydro-N-[4-[[4-(1-methylethoxy) phenyl]methyl] phenyl]-1H-imadazol-2-amine (a.k.a. JV-DE1), have been shown to decrease microvasculature leakage in anterior uveitis (Lin N and Bazan H, et al. 1991; Rubin R M, Samples J R, et al. 1988), decrease neovascularization and diminish eosinophil accumulation in corneal grafts (Cohen R A, Gebhardt B M, et al. 1992). So PAF antagonists could be useful in alleviating ocular inflammatory responses such as vascular permeability and chemotactic activity in the front, as well as in the back of the eye (Bazan H, Yao Y, et al. 1994). Ginkgo biloba extracts have neuroprotective properties under conditions such as hypoxia/ischemia, seizure activity and peripheral nerve damage. One of the components of Ginkgo biloba, ginkgolide B, is a potent platelet-activating factor (PAF) antagonist (Smith P, Maclennan K, et al, 1996).

### Pterygium

Provided herein is a method of treating pterygium in a patient by administering to the patient a therapeutically effective amount of 4,5-dihydro-N-[4-[[4-(1-methylethoxy) phenyl]methyl] phenyl]-1H-imadazol-2-amine. In some embodiments, the 4,5-dihydro-N-[4-[[4-(1-methylethoxy) phenyl] methyl] phenyl]-1H-imadazol-2-amine is administered topically to the surface of the eye. In some embodiments, the 4,5-dihydro-N-[4-[[4-(1-methylethoxy) phenyl]methyl] phenyl]-1H-imadazol-2-amine is administered periorbitally. In some embodiments, the 4,5-dihydro-N-[4-[[4-(1-methylethoxy) phenyl] methyl] phenyl]-1H-imadazol-2-amine is administered both periorbitally and topically to the surface of the eye. In some embodiments, treatment of pinguecula or pterygium comprises administering a therapeutically effective amount of 4,5-dihydro-N-[4-[[4-(1-methylethoxy) phenyl] methyl] phenyl]-1H-imadazol-2-amine and another anti-inflammatory agent.

Early in the disease process, pterygia are usually asymptomatic; however, there can be signs of dry eye (such as burning, itching or tearing) as the lesion causes irregular wetting of the ocular surface. A unique feature of the pterygium epithelial cell is its positive immunohistochemical staining for different types of matrix metalloproteinases that are absent in normal conjunctival, limbal or corneal cells (Krachmer, J. H. et al. 2005). Short-term use of topical corticosteroid eye drops may be used to reduce redness and inflammation. Where dryness of the eye is a problem, artificial tears are used to keep the eye well lubricated. There can be more severe symptoms such as redness (due to local tissue bleeding), swelling, and pain. In addition to the anti-inflammatory characteristics via VEGF suppression, IP receptor antagonists have been shown to be involved in bleeding suppression via inhibition of blood vessel dilatation.

One embodiment provides a method of treating pinguecula or pterygium comprising topical administration of a composition comprising an anti-inflammatory agent to the surface of the eye. In some embodiments, topical administration of an anti-inflammatory agent to the surface of the eye to treat pinguecula or pterygium is achieved through eye drops. In some embodiments, the anti-inflammatory agent is a non-steroidal anti-inflammatory agent. In another embodiment, the agent is a steroid, a COX inhibitor, or a prostanoid receptor inhibitor capable of blocking single or multiple receptors. In another embodiment, the agent is a corticoands, such as, but not limited to, cortisone, prednisolone, flurometholone, dexamethasone, medrysone, loteprednol fluazacort, hydrocortisone, prednisone, betamethasone, methylprednisolone, riamcinolone hexacatonide, paramethasone acetate, diflorasone, fluocinonide, fluocinolone, and triamcinolone. In another embodiment, the agent is a non-steroidal anti-inflammatory agent, such as aspirin, diclofenac, rofecoxib, ibuprofen, orindomethacin. One embodiment provides a method of treating pinguecula or pterygium comprising topical administration of a composition comprising an antagonist of the IP and/or PAF receptor to the surface of the eye. In some embodiments, topical administration of a composition comprising an antagonist of the IP and/or PAF receptor to the surface of the eye to treat pinguecula or pterygium is achieved through eye drops One embodiment provides a method of treating pinguecula orpterygium comprising topical administration of a composition comprising 4,5-dihydro-N-[4-[[4-(1-methylethoxy) phenyl] methyl] phenyl]-1H-imadazol-2-amine to the surface of the eye. In some embodiments, topical administration of 4,5-dihydro-N-[4-[[4-(1-methylethoxy) phenyl] methyl] phenyl]-1H-imadazol-2-amine to the surface of the eye is achieved using eye drops.

One embodiment provides a method of treating pinguecula or pterygium comprising administration to the eyelid of a composition comprising an anti-inflammatory agent. In some embodiments, embodiment, the anti-inflammatory agent is a non-steroidal anti-inflammatory agent. In another embodiment, the agent is a steroid, a COX inhibitor, or a prostanoid receptor inhibitor capable of blocking single or multiple receptors. In another embodiment, the agent is a corticoands, such as, but not limited to, cortisone, prednisolone, flurometholone, dexamethasone, medrysone, loteprednol fluazacort, hydrocortisone, prednisone, betamethasone, methylprednisolone, riamcinolonehexacatonide, paramethasone acetate, diflorasone, fluocinonide, fluocinolone, and triamcinolone. In another embodiment, the agent is a non-steroidal anti-inflammatory agent, such as aspirin, diclofenac, rofecoxib, ibuprofen, or indomethacin. One embodiment provides a method of treating pinguecula or pterygium comprising administration to the eyelid of a composition comprising an antagonist of the IP and/or PAF receptor. One embodiment provides a method of treating pinguecula or pterygium comprising administration to the eyelid of a composition comprising 4,5-dihydro-N-[4-[[4-(1-methylethoxy) phenyl] methyl] phenyl]-1H-imadazol-2-amine. In some embodiments, treatment of pinguecula or pterygium comprises administering a therapeutically effective amount of 4,5-dihydro-N-[4-[[4-(1-methylethoxy) phenyl] methyl] phenyl]-1H-imadazol-2-amine and another anti-inflammatory agent.

One embodiment provides a method of treating pinguecula or pterygium comprising periorbital administration of a composition comprising an anti-inflammatory agent. In some embodiments, the anti-inflammatory agent is a non-steroidal anti-inflammatory agent. In another embodiment, the agent is a steroid, a COX inhibitor, or a prostanoid receptor inhibitor capable of blocking single or multiple receptors. In another embodiment, the agent is a corticoands, such as, but not limited to, cortisone, prednisolone, flurometholone, dexamethasone, medrysone, loteprednol fluazacort, hydrocortisone, prednisone, betamethasone, methylprednisolone, riamcinolone hexacatonide, paramethasone acetate, diflorasone, fluocinonide, fluocinolone, and triamcinolone. In another embodiment, the agent is a non-steroidal anti-inflammatory agent, such as aspirin, diclofenac, rofecoxib, ibuprofen, or indomethacin. One embodiment provides a method of treating pinguecula orpterygium comprising periorbital administration of a composition comprising an antagonist of the IP and/or PAF receptor. One embodiment provides a method of treating pinguecula or pterygium comprising periorbital administration of a composition comprising 4,5-dihydro-N-[4-[[4-(1-methylethoxy) phenyl] methyl] phenyl]-1H-imadazol-2-amine. One embodiment provides a method of treating pinguecula or pterygium comprising periorbital administration of a composition comprising an omega-3 fatty acid or a pharmaceutically acceptable ester or salt thereof.

### Other Anterior Eye Diseases

Also provided herein are methods of treating additional disease of the anterior eye with 4,5-dihydro-N-[4-[[4-(1-methylethoxy) phenyl] methyl] phenyl]-1H-imadazol-2-amine. In some embodiments, the 4,5-dihydro-N-[4-[[4-(1-methylethoxy) phenyl] methyl] phenyl]-1H-imadazol-2-amine is administered topically to the surface of the eye to treat anterior eye diseases. In some embodiments, topical administration of 4,5-dihydro-N-[4-[[4-(1-methylethoxy) phenyl]methyl] phenyl]-1H-imadazol-2-amine to the surface of the eye to treat anterior eye disease is achieved using eye drops. In some embodiments, the 4,5-dihydro-N-[4-[[4-(1-methylethoxy) phenyl] methyl] phenyl]-1H-imadazol-2-amine is administered periorbitally. In some embodiments, the 4,5-dihydro-N-[4-[[4-(1-methylethoxy) phenyl] methyl] phenyl]-1H-imadazol-2-amine is administered to the eyelid of a subject. In some embodiments, the 4,5-dihydro-N-[4-[[4-(1-methylethoxy) phenyl] methyl] phenyl]-1H-imadazol-2-amine is administered through a combination of eye drops and periorbital or eyelid administration to treat anterior eye diseases.

Also provided herein are methods for treating diseases of the anterior eye with omega-3 fatty acid or a pharmaceutically acceptable ester or salt thereof. In some embodiments, the omega-3 fatty acid or a pharmaceutically acceptable ester or salt thereof is administered periorbitally to a patient to treat a disease or disorder of the anterior segment of the eye of the patient.

In some embodiments, the anterior eye disease or disorder is anterior segment dysgenesis, cataract, iritis, pterygium, keratoconjunctivitis, keratitis, conjunctivitis, keratoconus, ectatic disorders (including keratoglobus, pellucid marginal degeneration), Pseudophakic and aphakic bullous keratopathy, episcleritis, corneal ulceration, corneal dysplasia, corneal ulceration, Fuchs' endothelial dystrophy and other corneal dystrophies (including lattice, granular, macular, and map-dot fingerprint), ocular cicatricial pemphigoid, Stevens Johnson syndrome, acute and chronic uveitis (anterior uveitis, intermediate uveitis), trauma to the cornea, conjunctiva and anterior segment including iris trauma, penetrating ocular trauma.

In some embodiments, the anterior eye disease or disorder is dry eye, dry eye diseases, ocular discomfort, irritation, pain and stress, or chemical burns. In some embodiments, an omega-3 fatty acid or a pharmaceutically acceptable ester or salt thereof is administered periorbitally to a patient to treat dry eye, dry eye diseases, ocular discomfort, irritation, pain and stress, or chemical burns.

In some embodiments, the disease or disorder of the anterior region of the eye is a condition affecting the eyelid. In some embodiments, the eyelid condition is blepharitis, blepharospasm, chalazion, ptosis, coloboma, dermatochalasis, ectropion, entropion, trichiasis, stye, meibomianitis, Meibomian Gland Dysfunction, lacrimal gland obstruction, lacrimal gland obstruction, seborrheic keratitis, actinic keratitis, bacterial infection, or viral infection.

In some embodiments, the eyelid condition is treating by applying 4,5-dihydro-N-[4-[[4-(1-methylethoxy) phenyl] methyl] phenyl]-1H-imadazol-2-amine to the periorbital skin of the eye. In some embodiments, the eyelid condition is treating by applying 4,5-dihydro-N-[4-[[4-(1-methylethoxy) phenyl] methyl] phenyl]-1H-imadazol-2-amine to the eyelid of a subject. In some embodiments, the eyelid condition is treating by applying 4,5-dihydro-N-[4-[[4-(1-methylethoxy) phenyl] methyl] phenyl]-1H-imadazol-2-amine to the surface of the eye. In some embodiments, topical administration of 4,5-dihydro-N-[4-[[4-(1-methylethoxy) phenyl] methyl]phenyl]-1H-imadazol-2-amine to the surface of the eye to treat the eyelid condition is achieved using eye drops.

In some embodiments, the eyelid condition is treating by applying an omega-3 fatty acid or a pharmaceutically acceptable ester or salt thereof to the periorbital skin of the eye. In some embodiments, the eyelid condition is treating by applying an omega-3 fatty acid or a pharmaceutically acceptable ester or salt thereof to the eyelid of a subject.

### Diseases Affecting the Anterior and Posterior Segments of the Eye

Also provided herein are methods of treating certain diseases and disorders associated with both the anterior portion and posterior portion of the eye. Examples of such diseases include lymphatic malformations of the orbit (i.e., orbital lymphangiomas), Thyroid Eye Disease (Graves' Eye Disease), acute and chronic uveitis (including intermediate uveitis, posterior uveitis, panuveitis). In some embodiments, these diseases or disorders can be treated with 4,5-dihydro-N-[4-[[4-(1-methylethoxy) phenyl] methyl] phenyl]-1H-imadazol-2-amine. In some embodiments the 4,5-dihydro-N-[4-[[4-(1-methylethoxy) phenyl] methyl] phenyl]-1H-imadazol-2-amine may treat diseases of the anterior of the eye through administration to the surface of the eye (e.g., in a droplet formulation). In some embodiments, 4,5-dihydro-N-[4-[[4-(1-methylethoxy) phenyl] methyl] phenyl]-1H-imadazol-2-amine may treat diseases of the posterior of the eye through administration through the periorbital skin. In some embodiments, 4,5-dihydro-N-[4-[[4-(1-methylethoxy) phenyl] methyl] phenyl]-1H-imadazol-2-amine may treat diseases that affect the anterior and posterior segments of the eye through administration to the surface of the eye (e.g., in a droplet formulation) and administration to the periorbital skin. In some embodiments, these diseases or disorders can be treated with an omega-3 fatty acid or a pharmaceutically acceptable ester or salt thereof. In some embodiments, an omega-3 fatty acid or a pharmaceutically acceptable ester or salt thereof is administered through the periorbital skin to treat both the anterior and posterior segment of the eye of a patient. In some embodiments, these diseases or disorders can be treated with a combination of 4,5-dihydro-N-[4-[[4-(1-methylethoxy) phenyl] methyl] phenyl]-1H-imadazol-2-amine and an omega-3 fatty acid or a pharmaceutically acceptable ester or salt thereof. In some embodiments, 4,5-dihydro-N-[4-[[4-(1-methylethoxy) phenyl] methyl] phenyl]-1H-imadazol-2-amine and an omega-3 fatty acid or a pharmaceutically acceptable ester or salt thereof are formulated together as a single composition to be applied to the periorbital skin of a patient. In some embodiments, the 4,5-dihydro-N-[4-[[4-(1-methylethoxy) phenyl] methyl] phenyl]-1H-imadazol-2-amine is administered topically to the surface of the eye (e.g., in a droplet formulation), and the omega-3 fatty acid or a pharmaceutically acceptable ester or salt thereof is delivered by an alternative means, such as administration to the periorbital skin of a patient.

### Uveitis (Including Panuveitis)

Uveitis is caused by inflammatory responses inside the eye. Uveitis treatments primarily try to eliminate inflammation or alleviate pain. Anterior uveitis occurs in the front of the eye. It is the most common form of uveitis. Intermediate uveitis is commonly seen in young adults. The center of the inflammation often appears in the vitreous. Posterior uveitis is the least common form of uveitis. It primarily occurs in the back of the eye, often involving both the retina and the choroid. Treatments depend on the type of uveitis a patient displays. Some, such as using corticosteroid eye drops and injections around the eye or inside the eye, may exclusively target the eye whereas other treatments, such immunosuppressive agents taken by mouth, may be used when the disease is occurring in both eyes, particularly in the back of both eyes. Panuveitis involving both anterior and posterior ocular segments may also occur.

Also provided herein is a method of treating uveitis in a patient by administering to the patient a therapeutically effective amount of 4,5-dihydro-N-[4-[[4-(1-methylethoxy) phenyl] methyl] phenyl]-1H-imadazol-2-amine. In some embodiments, the 4,5-dihydro-N-[4-[[4-(1-methylethoxy) phenyl] methyl] phenyl]-1H-imadazol-2-amine is administered topically to the surface of the eye. In some embodiments, topical administration of 4,5-dihydro-N-[4-[[4-(1-methylethoxy) phenyl] methyl] phenyl]-1H-imadazol-2-amine to the surface of the eye is achieved using eye drops. In some embodiments, the 4,5-dihydro-N-[4-[[4-(1-methylethoxy) phenyl] methyl] phenyl]-1H-imadazol-2-amine is administered periorbitally. In some embodiments, the 4,5-dihydro-N-[4-[[4-(1-methylethoxy) phenyl] methyl] phenyl]-1H-imadazol-2-amine is administered through a combination of eye drops and periorbital administration.

Also provided herein is a method of treating uveitis in a patient by administering to the patient a therapeutically effective amount of an omega-3 fatty acid or a pharmaceutically ester or salt thereof. In some embodiments, the omega-3 fatty acid or a pharmaceutically ester or salt thereof is administered periorbitally. In some embodiments, the omega-3 fatty acid or a pharmaceutically ester or salt thereof is administered to the eyelid of a patient. In some embodiments, treatment of panuveitis or uveitis comprises administering a therapeutically effective amount of 4,5-dihydro-N-[4-[[4-(1-methylethoxy) phenyl] methyl] phenyl]-1H-imadazol-2-amine and an omega-3 fatty acid or a pharmaceutically acceptable ester or salt thereof.

One embodiment provides a method of treating panuveitis or uveitis comprising topical administration of a composition comprising an immunosuppressive agent to the surface of the eye. In some embodiments, topical administration of the immunosuppressive agent to the surface of the eye to treat panuveitis or uveitis is achieved through eye drops. In some embodiments, the immunosuppressive agent is methotrexate, mycophenolate, azathioprine, or cyclosporine.

One embodiment provides a method of treating panuveitis or uveitis comprising periorbital administration of a composition comprising an immunosuppressive agent. In some embodiments, the immunosuppressive agent is methotrexate, mycophenolate, azathioprine, or cyclosporine.

One embodiment provides a method of treating panuveitis or uveitis comprising administration to the eyelid of a composition comprising an immunosuppressive agent. In some embodiments, the immunosuppressive agent is methotrexate, mycophenolate, azathioprine, or cyclosporine. In some embodiments, treatment of panuveitis or uveitis comprises administering a therapeutically effective amount of 4,5-dihydro-N-[4-[[4-(1-methylethoxy) phenyl] methyl]phenyl]-1H-imadazol-2-amine and an immunosuppressive agent. In some embodiments, treatment of panuveitis or uveitis comprises administering a therapeutically effective amount of an omega-3 fatty acid or a pharmaceutically ester or salt thereof and an immunosuppressive agent. In some embodiments, treatment of panuveitis or uveitis comprises administering a therapeutically effective amount of 4,5-dihydro-N-[4-[[4-(1-methylethoxy) phenyl] methyl]phenyl]-1H-imadazol-2-amine, a therapeutically effective amount of an omega-3 fatty acid or pharmaceutically acceptable ester or salt thereof, and an immunosuppressive agent.

One embodiment provides a method of treating panuveitis or uveitis comprising topical administration of a composition comprising an anti-inflammatory agent to the surface of the eye. In some embodiments, topical administration of the anti-inflammatory agent to the surface of the eye to treat panuveitis or uveitis is achieved through eye drops. In some embodiments, the anti-inflammatory agent is a non-steroidal anti-inflammatory agent. In another embodiment, the agent is a steroid, a COX inhibitor, or a prostanoid receptor inhibitor capable of blocking single or multiple receptors. In another embodiment, the agent is a corticoands, such as, but not limited to, cortisone, prednisolone, flurometholone, dexamethasone, medrysone, loteprednol fluazacort, hydrocortisone, prednisone, betamethasone, methylprednisolone, riamcinolone hexacatonide, paramethasone acetate, diflorasone, fluocinonide, fluocinolone, and triamcinolone. In another embodiment, the agent is a non-steroidal anti-inflammatory agent, such as aspirin, diclofenac, rofecoxib, ibuprofen, or indomethacin.

One embodiment provides a method of treating panuveitis oruveitis comprising periorbital administration of a composition comprising an anti-inflammatory agent. In some embodiments, the anti-inflammatory agent is a non-steroidal anti-inflammatory agent. In another embodiment, the agent is a steroid, a COX inhibitor, or a prostanoid receptor inhibitor capable of blocking single or multiple receptors. In another embodiment, the agent is a corticoands, such as, but not limited to, cortisone, prednisolone, flurometholone, dexamethasone, medrysone, loteprednol fluazacort, hydrocortisone, prednisone, betamethasone, methylprednisolone, riamcinolone hexacatonide, paramethasone acetate, diflorasone, fluocinonide, fluocinolone, and triamcinolone. In another embodiment, the agent is a non-steroidal anti-inflammatory agent, such as aspirin, diclofenac, rofecoxib, ibuprofen, or indomethacin.

One embodiment provides a method of treating panuveitis or uveitis comprising administration to the eyelid of a composition comprising an anti-inflammatory agent. In some embodiments, the anti-inflammatory agent is a non-steroidal anti-inflammatory agent. In another embodiment, the agent is a steroid, a COX inhibitor, or a prostanoid receptor inhibitor capable of blocking single or multiple receptors. In another embodiment, the agent is a corticoands, such as, but not limited to, cortisone, prednisolone, flurometholone, dexamethasone, medrysone, loteprednol fluazacort, hydrocortisone, prednisone, betamethasone, methylprednisolone, riamcinolone hexacatonide, paramethasone acetate, diflorasone, fluocinonide, fluocinolone, and triamcinolone. In another embodiment, the agent is a non-steroidal anti-inflammatory agent, such as aspirin, diclofenac, rofecoxib, ibuprofen, or indomethacin. In some embodiments, treatment of panuveitis or uveitis comprises administering a therapeutically effective amount of 4,5-dihydro-N-[4-[[4-(1-methylethoxy) phenyl] methyl] phenyl]-1H-imadazol-2-amine and an anti-inflammatory agent. In some embodiments, treatment of panuveitis or uveitis comprises administering a therapeutically effective amount of an omega-3 fatty acid or pharmaceutically acceptable ester or salt thereof and an anti-inflammatory agent.

In some embodiments, treatment of panuveitis or uveitis comprises administering a therapeutically effective amount of 4,5-dihydro-N-[4-[[4-(1-methylethoxy) phenyl] methyl]phenyl]-1H-imadazol-2-amine, a therapeutically effective amount of an omega-3 fatty acid or pharmaceutically acceptable ester or salt thereof, and an anti-inflammatory agent.

### Thyroid Eye Disease

Thyroid Eye Disease may also be called Graves' Eye Disease, Graves' Opthalmopathy, or Graves' Orbitopathy. Thyroid eye diseaseusually develops in people with an overactive thyroid caused by Graves' disease. Graves' disease is an autoimmune disease caused by antibodies directed against receptors present in the thyroid cells and on the surface of the cells behind the eyes. Thyroid eye disease may result in a feeling of irritation or grittiness in the eyes, redness or inflammation of the conjunctiva, excessive tearing or dry eyes, swelling of the eyelids, sensitivity to light, forward displacement or bulging of the eyes (proptosis), and double vision. In more advanced cases of Thyroid Eye Disease, a patient may experience decreased eye movement, incomplete closure of the eye with corneal ulceration, compression of the optic nerve and rarely, loss of vision.

Thyroid Eye Disease treatments primarily try to eliminate inflammation or alleviate pain. Treatments may include applying cool compresses to a patient's eyes, wearing sunglasses, lubricating eyedrops, elevation of a patient's head, prism glasses, steroids, eyelid surgery, eye muscle surgery, orbital decompression surgery, or antibody to insulin-like growth factor-1 receptor (IGF-1R).

Also provided herein is a method of treating thyroid eye disease in a patient by administering to the patient a therapeutically effective amount of 4,5-dihydro-N-[4-[[4-(1-methylethoxy) phenyl] methyl] phenyl]-1H-imadazol-2-amine. In some embodiments, the 4,5-dihydro-N-[4-[[4-(1-methylethoxy) phenyl] methyl] phenyl]-1H-imadazol-2-amine is administered topically to the surface of the eye. In some embodiments, topical administration of 4,5-dihydro-N-[4-[[4-(1-methylethoxy) phenyl] methyl] phenyl]-1H-imadazol-2-amine to the surface of the eye is achieved using eye drops. In some embodiments, the 4,5-dihydro-N-[4-[[4-(1-methylethoxy) phenyl] methyl] phenyl]-1H-imadazol-2-amine is administered through a combination of eye drops and periorbital administration.

Also provided herein is a method of treating Thyroid Eye Disease in a patient by administering to the patient a therapeutically effective amount of an omega-3 fatty acid or a pharmaceutically ester or salt thereof. In some embodiments, the omega-3 fatty acid or a pharmaceutically ester or salt thereof is administered periorbitally. In some embodiments, the omega-3 fatty acid or a pharmaceutically ester or salt thereof is administered to the eyelid of a patient. In some embodiments, treatment of Thyroid Eye Disease comprises administering a therapeutically effective amount of 4,5-dihydro-N-[4-[[4-(1-methylethoxy) phenyl] methyl]phenyl]-1H-imadazol-2-amine and an omega-3 fatty acid or a pharmaceutically acceptable ester or salt thereof.

One embodiment provides a method of treating Thyroid Eye Disease comprising topical administration of a composition comprising an immunosuppressive agent to the surface of the eye. In some embodiments, topical administration of the immunosuppressive agent to the surface of the eye to treat Thyroid Eye Disease is achieved through eye drops. In some embodiments, the immunosuppressive agent is methotrexate, mycophenolate, azathioprine, or cyclosporine.

One embodiment provides a method of treating Thyroid Eye Disease comprising periorbital administration of a composition comprising an immunosuppressive agent. In some embodiments, the immunosuppressive agent is methotrexate, mycophenolate, azathioprine, or cyclosporine.

One embodiment provides a method of treating Thyroid Eye Disease comprising administration to the eyelid of a composition comprising an immunosuppressive agent. In some embodiments, immunosuppressive agent is methotrexate, mycophenolate, azathioprine, or cyclosporine. In some embodiments, treatment of Thyroid Eye Disease comprises administering a therapeutically effective amount of 4,5-dihydro-N-[4-[[4-(1-methylethoxy) phenyl] methyl]phenyl]-1H-imadazol-2-amine and an immunosuppressive agent. In some embodiments, treatment of Thyroid Eye Disease comprises administering a therapeutically effective amount of an omega-3 fatty acid or a pharmaceutically acceptable ester or salt thereof and an immunosuppressive agent. In some embodiments, treatment of Thyroid Eye Disease comprises administering a therapeutically effective amount of 4,5-dihydro-N-[4-[[4-(1-methylethoxy) phenyl] methyl] phenyl]-1H-imadazol-2-amine, a therapeutically effective amount of omega-3 fatty acid or a pharmaceutically acceptable ester or salt thereof, and an immunosuppressive agent.

One embodiment provides a method of treating Thyroid Eye Disease comprising topical administration of a composition comprising an anti-inflammatory agent to the surface of the eye. In some embodiments, topical administration of the anti-inflammatory agent to the surface of the eye to treat Thyroid Eye Disease is achieved through eye drops. In some embodiments, the anti-inflammatory agent is a non-steroidal anti-inflammatory agent. In another embodiment, the agent is a steroid, a COX inhibitor, or a prostanoid receptor inhibitor capable of blocking single or multiple receptors. In another embodiment, the agent is a corticoands, such as, but not limited to, cortisone, prednisolone, flurometholone, dexamethasone, medrysone, loteprednol fluazacort, hydrocortisone, prednisone, betamethasone, methylprednisolone, riamcinolone hexacatonide, paramethasone acetate, diflorasone, fluocinonide, fluocinolone, and triamcinolone. In another embodiment, the agent is a non-steroidal anti-inflammatory agent, such as aspirin, diclofenac, rofecoxib, ibuprofen, or indomethacin.

One embodiment provides a method of treating Thyroid Eye Disease comprising periorbital administration of a composition comprising an anti-inflammatory agent. In some embodiments, the anti-inflammatory agent is a non-steroidal anti-inflammatory agent. In another embodiment, the agent is a steroid, a COX inhibitor, or a prostanoid receptor inhibitor capable of blocking single or multiple receptors. In another embodiment, the agent is a corticoands, such as, but not limited to, cortisone, prednisolone, flurometholone, dexamethasone, medrysone, loteprednol fluazacort, hydrocortisone, prednisone, betamethasone, methylprednisolone, riamcinolone hexacatonide, paramethasone acetate, diflorasone, fluocinonide, fluocinolone, and triamcinolone. In another embodiment, the agent is a non-steroidal anti-inflammatory agent, such as aspirin, diclofenac, rofecoxib, ibuprofen, or indomethacin.

One embodiment provides a method of treating Thyroid Eye Disease comprising administration to the eyelid of a composition comprising an anti-inflammatory agent. In some embodiments, the anti-inflammatory agent is a non-steroidal anti-inflammatory agent. In another embodiment, the agent is a steroid, a COX inhibitor, or a prostanoid receptor inhibitor capable of blocking single or multiple receptors. In another embodiment, the agent is a corticoands, such as, but not limited to, cortisone, prednisolone, flurometholone, dexamethasone, medrysone, loteprednol fluazacort, hydrocortisone, prednisone, betamethasone, methylprednisolone, riamcinolone hexacatonide, paramethasone acetate, diflorasone, fluocinonide, fluocinolone, and triamcinolone. In another embodiment, the agent is a non-steroidal anti-inflammatory agent, such as aspirin, diclofenac, rofecoxib, ibuprofen, or indomethacin. In some embodiments, treatment of Thyroid Eye Disease comprises administering a therapeutically effective amount of 4,5-dihydro-N-[4-[[4-(1-methylethoxy) phenyl] methyl] phenyl]-1H-imadazol-2-amine and an anti-inflammatory agent. In some embodiments, treatment of Thyroid Eye Disease comprises administering a therapeutically effective amount of an omega-3 fatty acid or a pharmaceutically acceptable ester or salt thereof and an anti-inflammatory agent. In some embodiments, treatment of Thyroid Eye Disease comprises administering a therapeutically effective amount of 4,5-dihydro-N-[4-[[4-(1-methylethoxy) phenyl] methyl] phenyl]-1H-imadazol-2-amine, a therapeutically effective amount of omega-3 fatty acid or a pharmaceutically acceptable ester or salt thereof, and an anti-inflammatory agent.

### Orbital Lymphangiomas

Lymphangiomas, also known as lymphatic malformations, are multi-cystic, localized malformations that involve the lymphatic and vascular systems, most commonly occurring in the head and neck and are usually apparent at birth or by two years of age. Orbital Lymphangiomas characteristically involve the subconjunctival and periocular tissues. The lesions of the superficial or anterior orbital structures are usually diagnosed earlier. In a large number of patients, the lymphatic malformations have an activating mutation in the PIK3CA gene. PIK3CA is known to play a role in regulating cell growth by signaling through the PI3K/mTOR pathway. n-3 polyunsaturated fatty acids (PUFAs), namely, eicosapentaenoic acid (EPA) and docosahexaenoic acid (DHA), rapidly and efficiently suppress both mTOR complex 1 (mTORC1) and mTORC2 and their downstream signaling, and subsequently inhibit cell proliferation and angiogenesis while promoting apoptosis (Chen Z, et al., 2014, Oncogene, 33, 4548-4557). In additional to blepharoptosis (droopy eyelid), symptoms include swelling, intraorbital hemorrhage, ocular proptosis, cellulitis (redness, swelling, and pain in the affected area of the skin), vesicles in the conjunctiva (Wiegand et al., 2013). These are all typical symptoms of inflammation that can be suppressed by JV-DE1 and omega-3 polyunsaturated fatty acids.

Provided herein is a method of treating orbital lymphangiomas, or any inflammatory ocular disease, the method comprising administering 4,5-dihydro-N-[4-[[4-(1-methylethoxy) phenyl] methyl] phenyl]-1H-imadazol-2-amine to the eye of patient. In some embodiments, the 4,5-dihydro-N-[4-[[4-(1-methylethoxy) phenyl] methyl] phenyl]-1H-imadazol-2-amine is administered periorbitally. In some embodiments, the 4,5-dihydro-N-[4-[[4-(1-methylethoxy) phenyl] methyl] phenyl]-1H-imadazol-2-amine is administered topically to the surface of the eye (e.g., in a droplet formulation). In some embodiments, the 4,5-dihydro-N-[4-[[4-(1-methylethoxy) phenyl] methyl] phenyl]-1H-imadazol-2-amine is administered both periorbitally and topically to the surface of the eye.

Also provided herein is a method of treating orbital lymphangiomas, or any inflammatory ocular disease, the method comprising administering an omega-3 fatty acid or a pharmaceutically acceptable ester or salt thereof to the periorbital eye of patient. In some embodiments, an omega-3 fatty acid or a pharmaceutically acceptable ester or salt thereof is combined with 4,5-dihydro-N-[4-[[4-(1-methylethoxy) phenyl] methyl] phenyl]-1H-imadazol-2-amine to treat orbital lymphangiomas, or any other inflammatory ocular disease. In some embodiments, 4,5-dihydro-N-[4-[[4-(1-methylethoxy)phenyl] methyl] phenyl]-1H-imadazol-2-amine and an omega-3 fatty acid or a pharmaceutically acceptable ester or salt thereof are formulated together as a single composition to be applied to the periorbital skin of a patient. In some embodiments, the 4,5-dihydro-N-[4-[[4-(1-methylethoxy) phenyl] methyl] phenyl]-1H-imadazol-2-amine is administered topically to the surface of the eye (e.g., in a droplet formulation), and the omega-3 fatty acid or a pharmaceutically acceptable ester or salt thereof is delivered by an alternative means, such as administration to the periorbital skin of a patient. In some embodiments, the omega-3 fatty acid or a pharmaceutically acceptable ester or salt thereof is delivered to the periorbital skin of a patient.

Currently, the main therapeutic options for treating a lymphatic malformation are active observation, percutaneous drainage, surgery, sclerotherapy, laser therapy, radiofrequency ablation or medical therapy of oral drugs, sirolimus or sildenafil. Sclerotherapy is an umbrella term that characterizes the multiple types of agents that are injected (usually under ultrasound guidance) into the cystic spaces of the lesion, leading to scar formation and reduction in the size of the cyst and lesion. Specific agents used in sclerotherapy for treatment of orbital lymphangiomas include OK-432 (Picinibil), sodium tetradecyl sulfate, doxycycline, ethanol, pingyangmycin, and bleomycin.

Provided herein is a method of treating orbital lymphangiomas by administering a therapeutically effective amount of sodium tetradecyl sulfate or a salt, free acid, or ester thereof, to the periorbital skin of a patient. Also provided herein is a method of treating orbital lymphangiomas by administering a therapeutically effective amount of doxycycline or a salt, free acid, or ester thereof, to the periorbital skin of a patient. Also provided herein is a method of treating orbital lymphangiomas by administering a therapeutically effective amount of pingyangmycin or a salt, free acid, or ester thereof, to the periorbital skin of a patient. Also provided herein is a method of treating orbital lymphangiomas by administering a therapeutically effective amount of bleomycin or a salt, free acid, or ester thereof, to the periorbital skin of a patient.

Although the cause of Lymphangiomas is unknown, there is an activating mutation in the PIK3CA gene in a large number of patients. PIK3 CA is known to play a role in regulating cell growth by signaling through the PI3K/mTOR pathway. The most established mTOR inhibitors have shown tumor responses in clinical trials against various tumor types. Sirolimus, a mTOR inhibitor has been approved by FDA to treat lymphatic malformations via oral administration. Additionally, n-3 polyunsaturated fatty acids (PUFAs), such as omega-3, can abrogate the activity of mTORC1/2 pathways in vitro and in vivo, which have the potential for cancer prevention and tumor suppression (Chen et al., 2014). Provided herein is a method of treating orbital lymphangiomas by administering a therapeutically effective amount of an mTOR inhibitor to the periorbital skin of a patient. In some embodiments, the mTOR inhibitor is sirolimus, idelalisib, copanlisib, duvelisib, alpelisib, umbralisib, linperlisib, buparlisib, or BGB-10188. In some embodiments, a combination of a mTOR inhibitor and omega-3 fatty acids, or a pharmaceutically acceptable ester or salt thereof, is administrated to the periorbital skin of a patient.

Also provided herein is a method of treating orbital lymphangiomas by administering a therapeutically effective amount of a phosphodiesterase 5 (PDE-5) inhibitor to the periorbital skin of a patient. In some embodiments, the PDE-5 inhibitor is sildenafil. In some embodiments, a combination of a PDE-5 inhibitor and omega-3 fatty acids, or a pharmaceutically acceptable ester or salt thereof, is administrated to the periorbital skin of a patient.

### JV-DE1

A solution to treating certain ocular diseases has herein been achieved by administering of 4,5-dihydro-N-[4-[[4-(1-methylethoxy) phenyl] methyl] phenyl]-1H-imadazol-2-amine (JV-DE1) as eye-drops to the ocular surface or by application to the periorbital skin. Unexpectedly and surprisingly, application of 4,5-dihydro-N-[4-[[4-(1-methylethoxy) phenyl]methyl] phenyl]-1H-imadazol-2-amine to the periorbital skin provided much greater bioavailability to the vitreous humor and retina than was achieved by eye-drops.

The compound 4,5-dihydro-N-[4-[[4-(1-methylethoxy) phenyl] methyl] phenyl]-1H-imadazol-2-amine (a.k.a. JV-DE1, RO-1138452, CAY 10441) is of particular value in treating retinal diseases. It embodies two important pharmacological properties in a single molecule; it is both a prostanoid IP receptor antagonist and platelet activating factor (PAF) antagonist (Bley et al., 2006). At the vascular IP receptor 4,5-dihydro-N-[4-[[4-(1-methylethoxy) phenyl] methyl] phenyl]-1H-imadazol-2-amine had a pA2 of 8.2 (Jones R L et al. (2006) Br J Pharmacol 149: 110-120). At the PAF receptor, it was a high affinity antagonist with a pKi of 7.9 (Bley K R et al. (2006) Br J Pharmacol 147: 335-345). The incorporation of two distinct and diverse pharmacological properties in a single molecule is a distinct advantage (Woodward D F, Wang J W (2021) Trends Med. DOI: 10.15761/TiM.1000264) with respect to certain critical aspects of drug design; bioavailability, duration of action, formulation inter-drug compatibility, and cost of goods.

Combinations with JV-DE1

In treating various eye diseases or disorders, including disease or disorders of the posterior of the eye such as retinal diseases or posterior uveitis, 4,5-dihydro-N-[4-[[4-(1-methylethoxy) phenyl] methyl] phenyl]-1H-imadazol-2-amine (a.k.a. JV-DE1, RO-1138452, CAY 10441) can be combined with one or more additional therapeutic agents. The compound 4,5-dihydro-N-[4-[[4-(1-methylethoxy) phenyl] methyl] phenyl]-1H-imadazol-2-amine can be combined with one or more additional therapeutics, and this combination can be administered periorbitally, topically to the surface of the eye through eye drops, or topically to a subject's eyelid.

In some embodiments, a patient being treated with 4,5-dihydro-N-[4-[[4-(1-methylethoxy) phenyl] methyl] phenyl]-1H-imadazol-2-amine is further administered omega-3 fatty acids, including docosahexaenoic acid (DHA) and eicosapentaenoic acid (EPA).

In some embodiments, a patient being treated with 4,5-dihydro-N-[4-[[4-(1-methylethoxy) phenyl] methyl] phenyl]-1H-imadazol-2-amine is further administered a VEGF antibody, or a functional fragment thereof. In some embodiments, the administration of 4,5-dihydro-N-[4-[[4-(1-methylethoxy) phenyl] methyl] phenyl]-1H-imadazol-2-amine reduces the amount of VEGF antibody that would otherwise need to be administered to produce an intended therapeutic effect. In some embodiments, the reduced amount of VEGF antibody is manifested as a lower dose of VEGF antibody, or preferably, fewer or less frequent injections of the VEGF antibody (e.g., fewer injections into the eye of the patient). In some embodiments, the patient being treated with 4,5-dihydro-N-[4-[[4-(1-methylethoxy) phenyl] methyl] phenyl]-1H-imadazol-2-amine is administered a small molecule VEGF receptor antagonist in lieu of or in addition to the VEGF antibody.

In some embodiments, a patient being treated with 4,5-dihydro-N-[4-[[4-(1-methylethoxy) phenyl] methyl] phenyl]-1H-imadazol-2-amine is further administered a tyrosine kinase inhibitor.

In some embodiments, a patient being treated with 4,5-dihydro-N-[4-[[4-(1-methylethoxy) phenyl] methyl] phenyl]-1H-imadazol-2-amine is further administered a steroidal anti-inflammatory agent. In some embodiments, the steroidal anti-inflammatory again is selected from a group consisting of cortisone, prednisolone, methylprednisolone, raimcinolone, fluromethalone, medrysone, dexamethasone, lotprednol, hexacatonide, betamethasone, paramethasone, diflorasone, fluocinonide, fluocinolone, fluticasone, and triamcinolone.

In some embodiments, a patient being treated with 4,5-dihydro-N-[4-[[4-(1-methylethoxy) phenyl] methyl] phenyl]-1H-imadazol-2-amine is further administered a non-steroidal anti-inflammatory agent. In some embodiments, the non-steroidal anti-inflammatory agent is selected from a group consisting of ketorolac, nepafenac, amfenac, aspirin, indomethacin, flurbiprofen, ibuprofen, rofecoxib, and celecoxib.

In some embodiments, a patient being treated with 4,5-dihydro-N-[4-[[4-(1-methylethoxy) phenyl] methyl] phenyl]-1H-imadazol-2-amine is further administered an immunosuppressant.

In some embodiments, the immunosuppressantis selected from a group consisting of cyclosporine, liftegrast, methotrexate, azathioprine, inhibitors of the PI3K-AKT-mTOR signaling pathway (such as sirolimus, idelalisib, copanlisib, duvelisib, alpelisib, umbralisib, linperlisib, buparlisib, or BGB-10188), and agents that interfere with activation and function of the complement pathway (e.g. POT-4, ARC1905).

In some embodiments, the patient is co-administered 4,5-dihydro-N-[4-[[4-(1-methylethoxy) phenyl] methyl] phenyl]-1H-imadazol-2-amine and cyclosporine.

In some embodiments, the patient is co-administered 4,5-dihydro-N-[4-[[4-(1-methylethoxy) phenyl] methyl] phenyl]-1H-imadazol-2-amine and liftegrast.

In some embodiments, the patient is co-administered 4,5-dihydro-N-[4-[[4-(1-methylethoxy) phenyl] methyl] phenyl]-1H-imadazol-2-amine and methotrexate.

In some embodiments, the patient is co-administered 4,5-dihydro-N-[4-[[4-(1-methylethoxy) phenyl] methyl] phenyl]-1H-imadazol-2-amine and azathioprine. In some embodiments, the patient is co-administered 4,5-dihydro-N-[4-[[4-(1-methylethoxy) phenyl]methyl] phenyl]-1H-imadazol-2-amine, sirolimus, idelalisib, copanlisib, duvelisib, alpelisib, umbralisib, linperlisib, buparlisib, or BGB-10188.

In some embodiments, a patient being treated with 4,5-dihydro-N-[4-[[4-(1-methylethoxy) phenyl] methyl] phenyl]-1H-imadazol-2-amine is further administered a nicotinic anti-cholinergic agent. In some embodiments, the nicotinic anti-cholinergic agent is selected from a group consisting of hexamethonium, decamethonium, and mecamyline.

In some embodiments, a patient being treated with 4,5-dihydro-N-[4-[[4-(1-methylethoxy) phenyl] methyl] phenyl]-1H-imadazol-2-amine is further administered thalidomide.

In some embodiments, a patient being treated with 4,5-dihydro-N-[4-[[4-(1-methylethoxy) phenyl] methyl] phenyl]-1H-imadazol-2-amine is further administered a prostaglandin receptor antagonist. In some embodiments, the antagonist blocks multiple prostaglandin receptors. In some embodiments, the antagonist is AGN 211377 and AGN 225660.

In some embodiments, a patient being treated with 4,5-dihydro-N-[4-[[4-(1-methylethoxy) phenyl] methyl] phenyl]-1H-imadazol-2-amine is further administered a neuroprotective agent.

In some embodiments, the neuroprotective agent is selected from a group consisting of α₂-adrenoceptor agonists (e.g. brimonidine), NMDA antagonists (e.g. memantine), AMPA antagonists, Ca²⁺ blockers, σ-Irs-receptor agonists, pentazocine, endothelin receptor antagonists, Kinin antagonists, and anti-TNFα antibodies,

In some embodiments, a patient being treated with 4,5-dihydro-N-[4-[[4-(1-methylethoxy) phenyl] methyl] phenyl]-1H-imadazol-2-amine is further administered a neurotrophic/neuroregenerative agent (e.g ciliary neurotrophic factor, nerve growth factor, brain derived neurotrophic factor, 1 glial derived neurotrophic factor, neurotrophin 3), heat shock proteins, JNK inhibitors, synthetic bile acids (e.g. UDCA, TUDCA), progesterone, dopaminergics, neurotrophic factors, caspase inhibitors, acetyl-L-carnitine, acetylcholinesterase inhibitors, citicoline, acetylcysteine, retinoids (e.g. fenretinide), emixustat, anti-protein aggregation agents, phosphodiesterase inhibitors, nicotinamide, cannabinoids, citicholine, curcumin, minocycline, edaravone, erythropoietin, estrogen, L-theanine, melatonin, minocycline, noopept, pyrroloquinoline quinone, selegiline, simvastatin, esketamine, methylphenidate, ponesimod, glatiramer acetate, paliperidone, and vinpocetine agents that interferes with activation and function of the complement pathway, and vinpocetine.

In some embodiments, a patient being treated with 4,5-dihydro-N-[4-[[4-(1-methylethoxy) phenyl] methyl] phenyl]-1H-imadazol-2-amine is further administered an RNA interfering molecule.

In some embodiments, the RNA interfering molecule may be siRNA, miRNA, or shRNA.

In some embodiments the RNA interfering molecules is complementary to the gene sequence which encodes for a protein. In some embodiments, the RNA interfering molecule has a sequence that is at least partially complementary to the gene sequence, which encodes for a protein. In some embodiments, presence of the RNA interfering molecule produces silencing of the gene which encodes for a protein. In some embodiments the protein is a receptor. In some embodiments, a combination of at least two RNA interfering molecules are further administered to the patient. In some embodiments, at combination of at least two RNA interfering molecules silence the genes encoding for at least two proteins. In some embodiments the protein is an enzyme. In some embodiments the protein is selected from the group VEGF, PDGF, bFGF, SDF-1, HIF-1, PIGF, GLUT-1, Claudin cell adhesion molecules, HMBG-1, HuR, Ets1, GSK3β, RTP801, caspases 2-, 3-, 7-, PGC-1, ICAM1, t-PA, SNAI1, TBK1, ARMS2, TERT, ASK-1, and Nrf-2.

In some embodiments the RNA interfering molecule is a single stranded RNA. In some embodiments the RNA interfering molecule is a double stranded RNA. In some embodiments, the strand length of the RNA interfering molecule is about 10 nucleotides to about 200 nucleotides. In some embodiments, the strand length of the RNA interfering molecule is about 10 nucleotides to about 20 nucleotides, about 10 nucleotides to about 30 nucleotides, about 10 nucleotides to about 40 nucleotides, about 10 nucleotides to about 50 nucleotides, about 10 nucleotides to about 60 nucleotides, about 10 nucleotides to about 70 nucleotides, about 10 nucleotides to about 80 nucleotides, about 10 nucleotides to about 90 nucleotides, about 10 nucleotides to about 100 nucleotides, about 10 nucleotides to about 200 nu cleotides, about 20 nucleotides to about 30 nucleotides, about 20 nucleotides to about 40 nucleotides, about 20 nucleotides to about 50 nucleotides, about 20 nucleotides to about 60 nucleotides, about 20 nucleotides to about 70 nucleotides, about 20 nucleotides to about 80 nucleotides, about 20 nucleotides to about 90 nucleotides, about 20 nucleotides to about 100 nucleotides, about 20 nucleotides to about 200 nucleotides, about 30 nucleotides to about 40 nucleotides, about 30 nucleotides to about 50 nucleotides, about 30 nucleotides to about 60 nucleotides, about 30 nucleotides to about 70 nucleotides, about 30 nucleotides to about 80 nucleotides, about 30 nucleotides to about 90 nucleotides, about 30 nucleotides to about 100 nucleotides, about 30 nucleotides to about 200 nucleotides, about 40 nucleotides to about 50 nucleotides, about 40 nucleotides to about 60 nucleotides, about 40 nucleotides to about 70 nucleotides, about 40 nucleotides to about 80 nucleotides, about 40 nucleotides to about 90 nucleotide s, about 40 nucleotides to about 100 nucleotides, about 40 nucleotides to about 200 nucleotides, about 50 nucleotides to about 60 nucleotides, about 50 nucleotides to about 70 nucleotides, about 50 nucleotides to about 80 nucleotides, about 50 nucleotides to about 90 nucleotides, about 50 nucleotides to about 100 nucleotides, about 50 nucleotides to about 200 nucleotides, about 60 nucleotides to about 70 nucleotides, about 60 nucleotides to about 80 nucleotides, about 60 nucleotides to about 90 nucleotides, about 60 nucleotides to about 100 nucleotides, about 60 nucleotides to about 200 nucleotides, about 70 nucleotides to about 80 nucleotides, about 70 nucleotides to about 90 nucleotides, about 70 nucleotides to about 100 nucleotides, about 70 nucleotides to about 200 nucleotides, about 80 nucleotides to about 90 nucleotides, about 80 nucleotides to about 100 nucleotides, about 80 nucleotides to about 200 nucleotides, about 90 nucleotides to about 100 nucleotides, about 90 nucleotides to about 200 nucleotide s, or about 100 nucleotides to about 200 nucleotides. In some embodiments, the strand length of the RNA interfering molecule is about 10 nucleotides, about 20 nucleotides, about 30 nucleotides, about 40 nucleotides, about 50 nucleotides, about 60 nucleotides, about 70 nucleotides, about 80 nucleotides, about 90 nucleotides, about 100 nucleotides, or about 200 nucleotides. In some embodiments, the strand length of the RNA interfering molecule is at least about 10 nucleotides, about 20 nucleotides, about 30 nucleotides, about 40 nucleotides, about 50 nucleotides, about 60 nucleotides, about 70 nucleotides, about 80 nucleotides, about 90 nucleotides, or about 100 nucleotides. In some embodiments, the strand length of the RNA interfering molecule is at most about 20 nucleotides, about 30 nucleotides, about 40 nucleotides, about 50 nucleotides, about 60 nucleotides, about 70 nucleotides, about 80 nucleotides, about 90 nucleotides, about 100 nucleotides, or about 200 nucleotides.

In some embodiments the RNA interfering molecules may prevent expression of VEGF receptors or attenuate the biosynthesis of VEGF and its various isoforms.

In some embodiments, a patient being treated with 4,5-dihydro-N-[4-[[4-(1-methylethoxy) phenyl] methyl] phenyl]-1H-imadazol-2-amine is further administered an RNA interfering molecule selected from a group that reduces or abolishes receptor expression or reduces the biosynthesis of PDGF, bFGF, SDF-1, HIF-1, PIGF, GLUT-1, Claudin cell adhesion molecules, HMBG-1, HuR, Ets1, GSK3β, RTP801, caspases 2-, 3-, 7-, PGC-1, ICAM1, t-PA, SNAI1, TBK1, SRPK1, ClQ, HtrA1, ARMS2, TERT, ASK-1, and Nrf-2.

In some embodiments, a patient being treated with 4,5-dihydro-N-[4-[[4-(1-methylethoxy) phenyl] methyl] phenyl]-1H-imadazol-2-amine is further administered an anti-oxidant. In some embodiments, the anti-oxidant is selected from a group consisting of β-carotene, lutein, zeaxanthin, riboflavin, Niacin, and polyunsaturated fatty acids such as docosohexanoic acid (DHA), eicosapentanoic acid (EPA), vitamin B₃, vitamin B₆, vitamin B₉, vitamin B₁₂ vitamin C, vitamin E, CoQ10, ghrelin, α-lipoic acid, resveratrol, flavinoids, gingko bilbao extract, ICAPS R®, OFTAN MACULA®, and epigallocatechin-3-gallate.

In some embodiments, a patient being treated with 4,5-dihydro-N-[4-[[4-(1-methylethoxy) phenyl] methyl] phenyl]-1H-imadazol-2-amine is further administered a therapeutic antibody. In some embodiments, the therapeutic antibody is a PDGF, FGF, PIGF, SDF-1, or HIF-1 antibody. In some embodiments, the therapeutic antibody is an antibody that interferes with activation and function of the complement pathway. In some embodiments, the administration of 4,5-dihydro-N-[4-[[4-(1-methylethoxy) phenyl] methyl] phenyl]-1H-imadazol-2-amine reduces the amount of the therapeutic antibody that would otherwise need to be administered to produce an intended therapeutic effect. In some embodiments, the reduced amount of therapeutic antibody is manifested as a lower dose of therapeutic antibody, or preferably, fewer or less frequent injections of the therapeutic antibody (e.g., fewer injections into the eye of the patient).

In some embodiments, a patient being treated with 4,5-dihydro-N-[4-[[4-(1-methylethoxy) phenyl] methyl] phenyl]-1H-imadazol-2-amine is further administered a therapeutic epigenetic modulator of acylating, deacylating, methylating, or demethylating histone proteins.

In some embodiments, the treatment with 4,5-dihydro-N-[4-[[4-(1-methylethoxy) phenyl] methyl] phenyl]-1H-imadazol-2-amine is paired with an additional intervention, such as laser surgery or a steroid implant.

In some embodiments, the 4,5-dihydro-N-[4-[[4-(1-methylethoxy) phenyl]methyl] phenyl]-1H-imadazol-2-amine and the additional therapeutic agent are formulated together (e.g., as a single composition to be applied to the periorbital skin of a patient). In some embodiments, the additional therapeutic agent is delivered by an alternative means, such as injection, implant, or oral administration.

### Omega-3 Fatty Acids

The World Health Organization suggests that adults get 200 to 500 milligrams of omega-3 fatty acids, including docosahexaenoic acid (DHA) and eicosapentaenoic acid (EPA), per day for general health benefits. There are three main forms of omega-3 fatty acids: DHA and EPA, which are rich in cold-water fish oil, and alpha-linolenic acid (ALA), which is commonly derived from vegetable sources.

The retina contains a high concentration of DHA, which is not only important in maintenance of normal retinal integrity and visual function, but also plays an anti-inflammatory, antiapoptotic, neuroprotection role in the retina and brain. In addition to its nutritional, neuroprotective, and anti-oxidation properties, DHA is an important precursor for the resolvins and related compounds (e.g., protectins) through pathways involving cyclooxygenase and lipoxygenase enzymes which may resolve inflammatory responses in the retina and ocular surface, notably in the lacrimal gland where these DHA derivatives have been implicated in the pathogenesis of dry eye disease (Cortina and Bazan, 2011). Additionally, omega-3 fatty acids, including DHA, produce a local anesthetic effect. DHA has been shown to attenuate the nociceptive jaw-opening reflex in rats and may be a therapeutic agent and complementary alternative medicine for the prevention of acute trigeminal nociception or trigeminal neuralgia. DHA promotes the resolution of acute inflammation and potentially inhibits inflammatory and neuropathic pain.

Oral administration of mega doses of omega-3 fatty acids, including DHA, may benefit age-related macular degeneration (AMD), dry eye disease, retinitis pigmentosa, and retinopathy of prematurity. However, DHA is among the most difficult to orally consume in sufficient amounts for ocular benefits because it is contained in few food sources. Additionally, the oily property of these compounds makes them undesirable to put directly into the eye as eye drops. Therefore, there is a need for delivery methods capable of delivering therapeutically or preventively relevant amounts of omega-3 fatty acids to the tissue of the eye.

To overcome this problem, it has been surprisingly found that administration of omega-3 fatty acids, including DHA, when administered to the periorbital skin of the eye, provide substantial biodistribution in the tissues throughout the eye, including both the anterior and posterior portions of the eye.

In an aspect provided herein is a method of delivering one or more omega-3 fatty acids to the eye, including anterior and posterior portions, via periorbital skin administration. The omega-3 fatty acids can be derived from any suitable source. In some embodiments, the omega-3 fatty acid is isolated from fish tissue. The concentration of omega-3 in fish oil may be increased through ethylation. In some embodiments, the omega-3 fatty acid is isolated from a plant source. In some embodiments, the plant source of omega-3 fatty acid is algae, seaweed, nori, spirulina, or chlorella. In some embodiments, the plant source of omega-3 fatty acid is flaxseed oil.

In some embodiments, the omega-3 fatty acid is a C16 to C24 omega-3 fatty acid, or a combination of C16 to C24 omega-3 fatty acids. In some embodiments, the omega-3 fatty acid is a C18 to C22 omega-3 fatty acid, or a combination of C18 to C22 omega-3 fatty acids. In some embodiments, the omega-3 fatty acid is a very long chain monounsaturated fatty acid (VLCMUFA) or a very long chain polyunsaturated fatty acid (VLCPUFA).

In some embodiments, the omega-3 fatty acid is hexadecatrienoic acid (HTA), α-linolenic acid (ALA), stearidonic acid (SDA), eicosatrienoic acid (ETE), eicosatetraenoic acid (ETA), eicosapentaenoic acid (EPA), heneicosapentaenoic acid (HPA), docosapentaenoic acid (DPA), docosahexaenoic acid (DHA), tetracosapentaenoic acid, tetracosahexaenoic acid, or any combinationthereof. In some embodiments, the omega-3 fatty acid comprises tetraconsenoic acid, hexacosenoic acid, octacosenoic acid, or any combination thereof. In some embodiments, the omega-3 fatty acid comprises ALA, EPA, DHA, or any combination thereof. In some embodiments, the omega-3 fatty acid comprisesDHA. In some embodiments, the omega-3 fatty acid comprises EPA. In some embodiments, the omega-3 fatty acid comprises ALA. In some embodiments, the omega-3 fatty acid comprises both DHA and EPA.

In some embodiments, the omega-3 fatty acid is in the form of an omega-3 ethyl ester. Once in the skin, omega-3 ethyl esters can be converted by esterase to omega-3 free acid, which can easily pass the intercellular lipids of stratum corneum and hair pores. In some embodiments, the omega-3 fatty acid comprises a DHA ester. In some embodiments, the omega-3 fatty acid comprises an EPA ester. In some embodiments, the omega-3 fatty acid comprises a DHA ethyl ester. In some embodiments, the omega-3 fatty acid comprises an EPA ethyl ester. In some embodiments, the omega-3 fatty acid comprises an ester of both DHA and EPA. In some embodiments, the omega-3 fatty acid comprises an ethyl ester of both DHA and EPA. In some embodiments, the omega-3 fatty acid comprises omega-3-carboxylic acids (free fatty acids primarily composed of EPA and DHA). In some embodiments, the omega-3 fatty acid comprises icosapent ethyl (the ethyl ester of EPA).

In some embodiments, the omega-3 fatty acid is in the form of an omega-3 triglyceride. Natural fish oil contains the omega-3 fatty acids EPA and DHA mostly in the form of omega-3 triglycerides. Omega-3 triglycerides have a molecular weight around 900 Da. Once in the skin, omega-3 triglycerides can be converted by lipase to omega-3 free acid. Omega-3 triglycerides may also pass the intercellular lipids of stratum corneum and through hair pores.

In some embodiments, metabolites of omega-3 fatty acids may be administered to the periorbital skin of the eye to provide substantial biodistribution in the tissues throughout the eye, including both the anterior and posterior portions of the eye. In some embodiments, the omega-3 fatty acid metabolite may comprise a leukotriene or a derivative thereof. In some embodiments, the omega-3 fatty acid may comprise a lipoxin or a derivative thereof. In some embodiments, the omega-3 fatty acid metabolite may comprise a 5-series leukotriene (LTB5, LTC5, LTD5, LTE5). In some embodiments, the omega-3 fatty acid metabolite may comprise a prostanoid, such as a prostacyclin, thromboxane, or prostaglandin, or a derivative thereof. In some embodiments, the omega-3 fatty acid metabolite may comprise a 3-series prostanoid or prostaglandin. In some embodiments, the omega-3 fatty acid metabolite may comprise prostaglandin A3. In some embodiments, the omega-3 fatty acid metabolite may comprise prostaglandin 13. In some embodiments, the omega-3 fatty acid metabolite may comprise prostaglandin F2α. In some embodiments, the omega-3 fatty acid metabolite may comprise thromboxane A3. While EPA is great for helping lower chronic pain and inflammation anywhere in the body (for example: for cardiovascular health or diseases), DHA is best for the brain. To support brain health, the essential fatty acid supplement may have at least a ratio of 4:1 DHA to EPA. In some embodiments, the essential fatty acid supplement may have a ratio of 3:1 DHA to EPA. In some embodiments, the essential fatty acid supplement may have a ratio of 2:1 DHA to EPA. In some embodiments, the essential fatty acid supplement may have a ratio of 1:1 DHA to EPA. In some embodiments, the omega-3 fatty acid metabolite may comprise a maresin or a derivative thereof. In some embodiments, the omega-3 fatty acid metabolite may comprise a resolvin or a derivative thereof. In some embodiments, the omega-3 fatty acid metabolite may comprise a protectin or a derivative thereof.

In some embodiments, provided herein, is a method of promoting ocular health, preventing or treating ocular disease in a subject, the method comprising administering to the eye of the subject a composition comprising an omega-3 fatty acid, or a pharmaceutically acceptable ester or salt thereof. In some embodiments, promoting ocular health, preventing or treating ocular disease comprises treating or preventing age-related vision loss. In some embodiments, promoting ocular health, preventing or treating ocular disease comprises treating or preventing dry eye. In some embodiments, promoting ocular health, preventing or treating ocular disease comprises treating or preventing age-related macular degeneration. In some embodiments, promoting ocular health, preventing or treating ocular disease comprises treating or preventing dry age-related macular degeneration. In some embodiments, promoting ocular health comprises treating acute inflammation and neuropathic pain. In some embodiments, promoting ocular health comprises preventing acute trigeminal nociception or trigeminal neuralgia.

In some embodiments, provided herein, is a method of treating or preventing age-related vision loss in a subject, the method comprising administering to the eye of the subject a composition comprising an omega-3 fatty acid, or a pharmaceutically acceptable ester or salt thereof. In some embodiments, the composition comprising an omega-3 fatty acid, or a pharmaceutically acceptable ester or salt thereof is administered to the periorbital skin of the subject. In some embodiments, the composition comprising an omega-3 fatty acid, or a pharmaceutically acceptable ester or salt thereof is administered to the eyelid of a patient.

In some embodiments, provided herein, is a method of treating or preventing dry eye in a subject, the method comprising administering to the eye of the subject a composition comprising an omega-3 fatty acid, or a pharmaceutically acceptable ester or salt thereof. In some embodiments, the method comprises administering to the eye of the subject a composition comprising hyaluronic acid, hyaluronate, or a pharmaceutically acceptable ester or salt thereof. In some embodiments, the method comprises administering to the eye of the subject a composition comprising an omega-3 fatty acid and hyaluronic acid, hyaluronate, or a pharmaceutically acceptable ester or salt thereof. In some embodiments, the composition comprising an omega-3 fatty acid or a pharmaceutically acceptable ester or salt thereof is administered to the periorbital skin of the subject. In some embodiments, the composition comprising an omega-3 fatty acid or a pharmaceutically acceptable ester or salt thereof is administered to the eyelid of a patient.

In some embodiments, provided herein, is a method of treating or preventing age-related macular degeneration in a subject, the method comprising administering to the eye of the subject a composition comprising an omega-3 fatty acid, or a pharmaceutically acceptable ester or salt thereof. In some embodiments, the composition comprising an omega-3 fatty acid, or a pharmaceutically acceptable ester or salt thereof is administered to the periorbital skin of the subject. In some embodiments, the composition comprising an omega-3 fatty acid, or a pharmaceutically acceptable ester or salt thereof is administered to the eyelid of a patient.

In some embodiments, provided herein, is a method of treating or preventing dry age-related macular degeneration in a subject, the method comprising administering to the eye of the subject a composition comprising an omega-3 fatty acid, or a pharmaceutically acceptable ester or salt thereof. In some embodiments, the composition comprising an omega-3 fatty acid, or a pharmaceutically acceptable ester or salt thereof is administered to the periorbital skin of the subject. In some embodiments, the composition comprising an omega-3 fatty acid, or a pharmaceutically acceptable ester or salt thereof is administered to the eyelid of a patient.

Combinations with Omega-3 Fatty Acids

In treating various eye diseases or disorders, including disease or disorders of the posterior of the eye such as retinal diseases or posterior uveitis, an omega-3 fatty acid or a pharmaceutically acceptable ester or salt thereof (DHA, EPA, etc.) can be combined with one or more additional therapeutic agents. An omega-3 fatty acid or a pharmaceutically acceptable ester or salt thereof can be combined with one or more additional therapeutics, and this combination can be administered periorbitally or topically to a subject's eyelid.

In some embodiments, a patient being treated with an omega-3 fatty acid or a pharmaceutically acceptable ester or salt thereof is further administered 4,5-dihydro-N-[4-[[4-(1-methylethoxy) phenyl] methyl] phenyl]-1H-imadazol-2-amine or a pharmaceutically acceptable ester or salt thereof.

In some embodiments, a patient being treated with an omega-3 fatty acid or a pharmaceutically acceptable ester or salt thereof is further administered a VEGF antibody, or a functional fragment thereof.

In some embodiments, a patient being treated with an omega-3 fatty acid or a pharmaceutically acceptable ester or salt thereof is further administered a tyrosine kinase inhibitor.

In some embodiments, a patient being treated with an omega-3 fatty acid or a pharmaceutically acceptable ester or salt thereof is further administered a steroidal anti-inflammatory agent. In some embodiments, the steroidal anti-inflammatory again is selected from a group consisting of cortisone, prednisolone, methylprednisolone, raimcinolone, fluromethalone, medrysone, dexamethasone, lotprednol, hexacatonide, betamethasone, paramethasone, diflorasone, fluocinonide, fluocinolone, fluticasone, and triamcinolone.

In some embodiments, a patient being treated with an omega-3 fatty acid or a pharmaceutically acceptable ester or salt thereof is further administered a non-steroidal anti-inflammatory agent. In some embodiments, the non-steroidal anti-inflammatory agent is selected from a group consisting of ketorolac, nepafenac, amfenac, aspirin, indomethacin, flurbiprofen, ibuprofen, rofecoxib, and celecoxib.

In some embodiments, a patient being treated with an omega-3 fatty acid or a pharmaceutically acceptable ester or salt thereof is further administered an immunosuppressant. In some embodiments, the immunosuppressant is selected from a group consisting of cyclosporine, liftegrast, methotrexate, azathioprine, inhibitors of the PI3K-AKT-mTOR signaling pathway, (such as sirolimus, idelalisib, copanlisib, duvelisib, alpelisib, umbralisib, linperlisib, buparlisib, or BGB-10188), and agents that interfere with activation and function of the complement pathway (e.g. POT-4, ARC1905).

In some embodiments, the patient is co-administered an omega-3 fatty acid or a pharmaceutically acceptable ester or salt thereof and cyclosporine.

In some embodiments, the patient is co-administered an omega-3 fatty acid or a pharmaceutically acceptable ester or salt thereof and liftegrast.

In some embodiments, the patient is co-administered an omega-3 fatty acid or a pharmaceutically acceptable ester or salt thereof and methotrexate.

In some embodiments, the patient is co-administered an omega-3 fatty acid or a pharmaceutically acceptable ester or salt thereof and azathioprine. In some embodiments, the patient is co-administered an omega-3 fatty acid or a pharmaceutically acceptable ester or salt thereof and inhibitors of the PI3K-AKT-mTOR signaling pathway (such as sirolimus, idelalisib, copanlisib, duvelisib, alpelisib, umbralisib, linperlisib, buparlisib, or BGB-10188).

In some embodiments, a patient being treated with an omega-3 fatty acid or a pharmaceutically acceptable ester or salt thereof is further administered a nicotinic anti-cholinergic agent. In some embodiments, the nicotinic anti-cholinergic agent is selected from a group consisting of hexamethonium, decamethonium, and mecamyline.

In some embodiments, a patient being treated with an omega-3 fatty acid or a pharmaceutically acceptable ester or salt thereof is further administered thalidomide.

In some embodiments, a patient being treated with an omega-3 fatty acid or a pharmaceutically acceptable ester or salt thereof is further administered a prostaglandin receptor antagonist. In some embodiments, the antagonist blocks multiple prostaglandin receptors. In some embodiments, the antagonist is AGN 211377 and AGN 225660.

In some embodiments, a patient being treated with an omega-3 fatty acid or a pharmaceutically acceptable ester or salt thereof is further administered a neuroprotective agent. In some embodiments, the neuroprotective agent is selected from a group consisting of α₂-adrenoceptor agonists (e.g. brimonidine), NMDA antagonists (e.g. memantine), AMPA antagonists, Ca²⁺ blockers, σ-Irs-receptor agonists, pentazocine, endothelin receptor antagonists, Kinin antagonists, and anti-TNFα antibodies,

In some embodiments, a patient being treated with an omega-3 fatty acid or a pharmaceutically acceptable ester or salt thereof is further administered a neurotrophic/neuroregenerative agent (e.g ciliary neurotrophic factor, nerve growth factor, brain derived neurotrophic factor, 1 glial derived neurotrophic factor, neurotrophin 3), heat shock proteins, JNK inhibitors, synthetic bile acids (e.g. UDCA, TUDCA), progesterone, dopaminergics, neurotrophic factors, caspase inhibitors, acetyl-L-carnitine, acetylcholinesterase inhibitors, citicoline, acetylcysteine, retinoids (e.g. fenretinide), emixustat, anti-protein aggregation agents, phosphodiesterase inhibitors, nicotinamide, cannabinoids, citicholine, curcumin, minocycline, edaravone, erythropoietin, estrogen, L-theanine, melatonin, minocycline, noopept, pyrroloquinoline quinone, selegiline, simvastatin, esketamine, methylphenidate, ponesimod, glatiramer acetate, paliperidone, and vinpocetine agents that interferes with activation and function of the complement pathway, and vinpocetine.

In some embodiments, a patient being treated with an omega-3 fatty acid or a pharmaceutically acceptable ester or salt thereof is further administered an RNA interfering molecule. In some embodiments, the RNA interfering molecule may be siRNA, miRNA, or shRNA. In some embodiments the RNA interfering molecules is complementary to the gene sequence which encodes for a protein. In some embodiments, the RNA interfering molecule has a sequence that is at least partially complementary to the gene sequence, which encodes for a protein. In some embodiments, presence of the RNA interfering molecule produces silencing of the gene which encodes for a protein. In some embodiments the protein is a receptor. In some embodiments, a combination of at least two RNA interfering molecules are further administered to the patient. In some embodiments, at combination of at least two RNA interfering molecules silence the genes encoding for at least two proteins. In some embodiments the protein is an enzyme. In some embodiments the protein is selected from the group VEGF, PDGF, bFGF, SDF-1, HIF-1, PIGF, GLUT-1, Claudin cell adhesion molecules, HMBG-1, HuR, Ets1, GSK3β, RTP801, caspases 2-, 3-, 7-, PGC-1, ICAM1, t-PA, SNAI1, TBK1, ARMS2, TERT, ASK-1, and Nrf-2.

In some embodiments the RNA interfering molecule is a single stranded RNA. In some embodiments the RNA interfering molecule is a double stranded RNA. In some embodiments, the strand length of the RNA interfering molecule is about 10 nucleotides to about 200 nucleotides. In some embodiments, the strand length of the RNA interfering molecule is about 10 nucleotides to about 20 nucleotides, about 10 nucleotides to about 30 nucleotides, about 10 nucleotides to about 40 nucleotides, about 10 nucleotides to about 50 nucleotides, about 10 nucleotides to about 60 nucleotides, about 10 nucleotides to about 70 nucleotides, about 10 nucleotides to about 80 nucleotides, about 10 nucleotides to about 90 nucleotides, about 10 nucleotides to about 100 nucleotides, about 10 nucleotides to about 200 nucleotides, about 20 nucleotides to about 30 nucleotides, about 20 nucleotides to about 40 nucleotides, about 20 nucleotides to about 50 nucleotides, about 20 nucleotides to about 60 nucleotides, about 20 nucleotides to about 70 nucleotides, about 20 nucleotides to about 80 nucleotides, about 20 nucleotides to about 90 nucleotides, about 20 nucleotides to about 100 nucleotides, about 20 nucleotides to about 200 nucleotides, about 30 nucleotides to about 40 nucleotides, about 30 nucleotides to about 50 nucleotides, about 30 nucleotides to about 60 nucleotides, about 30 nucleotides to about 70 nucleotides, about 30 nucleotides to about 80 nucleotides, about 30 nucleotides to about 90 nucleotides, about 30 nucleotides to about 100 nucleotides, about 30 nucleotides to about 200 nucleotides, about 40 nucleotides to about 50 nucleotides, about 40 nucleotides to about 60 nucleotides, about 40 nucleotides to about 70 nucleotides, about 40 nucleotides to about 80 nucleotides, about 40 nucleotides to about 90 nucleotides, about 40 nucleotides to about 100 nucleotides, about 40 nucleotides to about 200 nucleotides, about 50 nucleotides to about 60 nucleotides, about 50 nucleotides to about 70 nucleotides, about 50 nucleotides to about 80 nucleotides, about 50 nucleotides to about 90 nucleotides, about 50 nucleotides to about 100 nucleotides, about 50 nucleotides to about 200 nucleotides, about 60 nucleotides to about 70 nucleotides, about 60 nucleotides to about 80 nucleotides, about 60 nucleotides to about 90 nucleotides, about 60 nucleotides to about 100 nucleotides, about 60 nucleotides to about 200 nucleotides, about 70 nucleotides to about 80 nucleotides, about 70 nucleotides to about 90 nucleotides, about 70 nucleotides to about 100 nucleotides, about 70 nucleotides to about 200 nucleotides, about 80 nucleotides to about 90 nucleotides, about 80 nucleotides to about 100 nucleotides, about 80 nucleotides to about 200 nucleotides, about 90 nucleotides to about 100 nucleotides, about 90 nucleotides to about 200 nucleotides, or about 100 nucleotides to about 200 nucleotides. In some embodiments, the strand length of the RNA interfering molecule is about 10 nucleotides, about 20 nucleotides, about 30 nucleotides, about 40 nucleotides, about 50 nucleotides, about 60 nucleotides, about 70 nucleotides, about 80 nucleotides, about 90 nucleotides, about 100 nucleotides, or about 200 nucleotides. In some embodiments, the strand length of the RNA interfering molecule is at least about 10 nucleotides, about 20 nucleotides, about 30 nucleotides, about 40 nucleotides, about 50 nucleotides, about 60 nucleotides, about 70 nucleotides, about 80 nucleotides, about 90 nucleotides, or about 100 nucleotides. In some embodiments, the strand length of the RNA interfering molecule is at most about 20 nucleotides, about 30 nucleotides, about 40 nucleotides, about 50 nucleotides, about 60 nucleotides, about 70 nucleotides, about 80 nucleotides, about 90 nucleotides, about 100 nucleotides, or about 200 nucleotides.

In some embodiments the RNA interfering molecules may prevent expression of VEGF receptors or attenuate the biosynthesis of VEGF and its various isoforms.

In some embodiments, a patient being treated with an omega-3 fatty acid or a pharmaceutically acceptable ester or salt thereof is further administered an RNA interfering molecule selected from a group that reduces or abolishes receptor expression or reduces the biosynthesis of PDGF, bFGF, SDF-1, HIF-1, PIGF, GLUT-1, Claudin cell adhesion molecules, HMBG-1, HuR, Ets1, GSK3β, RTP801, caspases 2-, 3-, 7-, PGC-1, ICAM1, t-PA, SNAI1, TBK1, SRPK1, CiQ, HtrA1, ARMS2, TERT, ASK-1, and Nrf-2.

In some embodiments, a patient being treated with an omega-3 fatty acid or a pharmaceutically acceptable ester or salt thereof is further administered an anti-oxidant. In some embodiments, the anti-oxidant is selected from a group consisting of β-carotene, lutein, zeaxanthin, riboflavin, Niacin, and polyunsaturated fatty acids such as docosohexanoic acid (DHA), eicosapentanoic acid (EPA), vitamin B₃, vitamin B₆, vitamin B₉, vitamin B₁₂, vitamin C, vitamin E, CoQ10, ghrelin, α-lipoic acid, resveratrol, flavinoids, gingko bilbao extract, ICAPS R®, OFTAN MACULA®, and epigallocatechin-3-gallate.

In some embodiments, a patient being treated with an omega-3 fatty acid or a pharmaceutically acceptable ester or salt thereof is further administered a therapeutic antibody. In some embodiments, the therapeutic antibody is a PDGF, FGF, PIGF, SDF-1, or HIF-1 antibody. In some embodiments, the therapeutic antibody is an antibody that interferes with activation and function of the complement pathway. In some embodiments, the administration of an omega-3 fatty acid or a pharmaceutically acceptable ester or salt thereof reduces the amount of the therapeutic antibody that would otherwise need to be administered to produce an intended therapeutic effect. In some embodiments, the reduced amount of therapeutic antibody is manifested as a lower dose of therapeutic antibody, or preferably, fewer or less frequent injections of the therapeutic antibody (e.g., fewer injections into the eye of the patient).

In some embodiments, a patient being treated with an omega-3 fatty acid or a pharmaceutically acceptable ester or salt thereof is further administered a therapeutic epigenetic modulator of acylating, deacylating, methylating, or demethylating histone proteins.

In some embodiments, the treatment with an omega-3 fatty acid or a pharmaceutically acceptable ester or salt thereof is paired with an additional intervention, such as laser surgery or a steroid implant.

In some embodiments, the omega-3 fatty acid or a pharmaceutically acceptable ester or salt thereof and the additional therapeutic agent are formulated together (e.g., as a single composition to be applied to the periorbital skin of a patient). In some embodiments, the additional therapeutic agent is delivered by an alternative means, such as injection, implant, or oral administration.

### Combinations of JV-DEJ and Omega-3 Fatty Acids

In treating various eye diseases or disorders, including disease or disorders of the anterior or posterior of the eye, 4,5-dihydro-N-[4-[[4-(1-methylethoxy) phenyl] methyl] phenyl]-1H-imadazol-2-amine can be combined with an omega-3 fatty acid or a pharmaceutically acceptable ester or salt thereof. The composition comprising 4,5-dihydro-N-[4-[[4-(1-methylethoxy) phenyl] methyl] phenyl]-1H-imadazol-2-amine and an omega-3 fatty acid or a pharmaceutically acceptable ester or salt thereof can be combined with one or more additional therapeutic agents. In some embodiments, 4,5-dihydro-N-[4-[[4-(1-methylethoxy) phenyl]methyl] phenyl]-1H-imadazol-2-amine and an omega-3 fatty acid or a pharmaceutically acceptable ester or salt thereof are formulated together as a single composition to be applied to the periorbital skin of a patient. In some embodiments, the 4,5-dihydro-N-[4-[[4-(1-methylethoxy) phenyl] methyl] phenyl]-1H-imadazol-2-amine is administered topically to the surface of the eye (e.g., in a droplet formulation), and the omega-3 fatty acid or a pharmaceutically acceptable ester or salt thereof is delivered by an alternative means, such as administration to the periorbital skin of a patient.

### Other Active Ingredients and Excipients

In one aspect, provided herein, a pharmaceutical composition suitable for topical periorbital administration may comprise any pharmaceutically acceptable excipient. In some embodiments, the pharmaceutically acceptable excipient comprises one or more acids, bases, electrolytes, buffers, solutes, antioxidants, stabilizers, and if required, preservatives. In some embodiments, the pharmaceutically acceptable excipient comprises a semifluorinated alkane. In some embodiments, the pharmaceutically acceptable excipient comprises perfluorohexyloctane. In some embodiments, the pharmaceutically acceptable excipient comprises perfluorobutylpentane. In some embodiments, a pharmaceutical composition suitable for topical periorbital administration comprises an omega-3 fatty acid or a pharmaceutically acceptable ester or salt thereof and a semifluorinated alkane. In some embodiments, a pharmaceutical composition suitable for topical periorbital administration comprises an omega-3 fatty acid or a pharmaceutically acceptable ester or salt thereof and perfluorohexyloctane. In some embodiments, a pharmaceutical composition suitable for topical periorbital administration comprises an omega-3 fatty acid or a pharmaceutically acceptable ester or salt thereof and perfluorobutylpentane. In some embodiments, a pharmaceutical composition suitable for topical periorbital administration comprises 4,5-dihydro-N-[4-[[4-(1-methylethoxy) phenyl] methyl]phenyl]-1H-imadazol-2-amine and a semifluorinated alkane. In some embodiments, a pharmaceutical composition suitable for topical periorbital administration comprises 4,5-dihydro-N-[4-[[4-(1-methylethoxy) phenyl] methyl] phenyl]-1H-imadazol-2-amine and perfluorohexyloctane. In some embodiments, a pharmaceutical composition suitable for topical periorbital administration comprises 4,5-dihydro-N-[4-[[4-(1-methylethoxy) phenyl] methyl]phenyl]-1H-imadazol-2-amine and perfluorobutylpentane. In some embodiments, a pharmaceutical composition suitable for topical periorbital administration comprises 4,5-dihydro-N-[4-[[4-(1-methylethoxy) phenyl] methyl] phenyl]-1H-imadazol-2-amine, an omega-3 fatty acid, and a semifluorinated alkane. In some embodiments, a pharmaceutical composition suitable for topical periorbital administration comprises 4,5-dihydro-N-[4-[[4-(1-methylethoxy) phenyl]methyl] phenyl]-1H-imadazol-2-amine, an omega-3 fatty acid, and perfluorohexyloctane. In some embodiments, a pharmaceutical composition suitable for topical periorbital administration comprises 4,5-dihydro-N-[4-[[4-(1-methylethoxy) phenyl] methyl] phenyl]-1H-imadazol-2-amine, an omega-3 fatty acid, and perfluorobutylpentane.

### Periorbital Skin Administration

In the treatment of retinal diseases, drugs applied topically as eye-drops have been known not to achieve the targeted tissue or are bioavailable only at pharmacologically inadequate concentrations in the posterior segment of the eye. While success has been claimed according to studies in small rodents with tiny eyes, these successes do not translate into species with eyes of similar dimensions to that of human eyes (del Amo et al., 2017). Where success has been reported from human-size eyes, it has resulted from inadequate experimental design. The drug per se must be directly detected and quantified, not estimated from techniques involving histology. Following eye-drop administration, drug is often absorbed into the bloodstream and thereby delivered to the retina. Plasma or blood levels must be monitored and reported. Further, satisfactory retinal bio-disposition can only be achieved by adequate tissue sampling. Drug residence should be established in the vitreous humor, neural retina, and the underlying posterior sclera. There should be no cross-contamination during tissue sampling. Systemic blood levels of the drug must be measured. In addition to the aforementioned considerations, the final imperative is that the drug is administered unilaterally. Eye-drops containing the drug formulation must be applied to one eye and not applied to the contralateral eye as a control. Full representation of the results from drug treated and untreated control eyes and plasma/blood must be reported numerically at all measured time points. If the drug levels in the ocular posterior segment tissues of the treated and untreated eyes are within experimental error, then retinal bioavailability is the result of absorption from the systemic circulation (blood-borne delivery).

In those cases where drug bioavailability in the retina and vitreous humor has been claimed, there is suboptimal experimental design. The following provide examples of common experimental deficiencies. (1) Acheampong A A et al. (2002) Drug Metab Disp 30: 421-429: sampling cross-contamination (del Amo et al., 2017); (2) U.S. Pat. No. 6,242,442, 2001: bilateral topical administration, blood levels not reported. (3) U.S. Pat. No. 9,446,026, 2016: bilateral ocular dosing; (4) Hu S, Koevery S (2016) J Ocular Pharmacol Ther 32: 203-210: rat eyes; (5) Kadam R S et al. (2011) Drug Metab Disp 39: 1529-1537: untreated eye as control absent; (6) Kiuchi K et al. (2008) Invest Ophthalmol Vis Sci 49: 1705-1711: drug levels not measured, only biological effect in mouse eyes after topical application; (7) Chastain J E et al. (2016) Exp Eye Res 145: 58-67: inadequate data reporting from contralateral control eye and plasma.

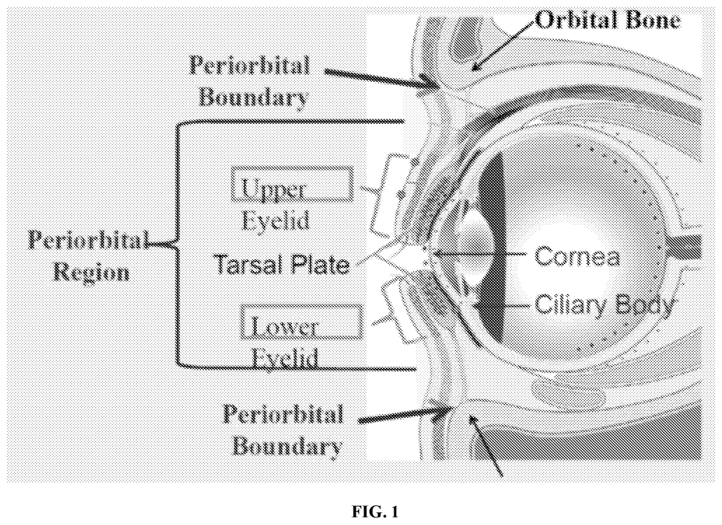

Thus, there exists a need for improved delivery methods for compounds to treat diseases at the posterior portion of the eye. Surprisingly, it has been found that periorbital administration of various compounds, including 4,5-dihydro-N-[4-[[4-(1-methylethoxy) phenyl]methyl] phenyl]-1H-imadazol-2-amine and omega-3 fatty acids, provides substantial biodistribution of the compounds throughout the eye. Moreover, application to the periorbital skin provides route of retinal delivery, via the vitreous humor, is not impeded by the retinal pigmented epithelium, Bruch's membrane and the blood retinal barrier. FIG. 1 shows a representation of ocular anatomy, including the periorbital region overlying the globe of the eye.

Application of compounds to the periorbital skin would provide drug delivery through the sclera pathway to the sclera and retina for the purpose of preventing the global elongation that may be associated with myopia. The periorbital skin route of delivery may be particularly advantageous in children, who represent the largest myopia patient population, since the unwanted nociceptive effects and inconvenience of eye-drops would be avoided.

In addition to oral administration, topical eye-drop is one of the only two currently available non-invasive mechanisms for ocular delivery. Of the topical eye-drop administration, there are potentially two pathways to deliver drugs to the posterior ocular segment: firstly, the “sclera pathway”, where a drug diffuses from the ocular surface to the conjunctiva, through the scleral water channels to reach the retina; secondly, the “cornea pathway”, where a drug penetrates the corneal surface, aqueous humor, lens/iris/ciliary body, vitreous humor, and then reaches the retina. Although there are multiple publications reporting topical delivery of potential therapeutics at pharmaceutical effective doses to the back of the eye in small animal models, these have not been successfully translated to larger species with relevant ocular anatomy, physiology and size of eyeball similar to those of human. Oral administration is another currently available way of non-invasively transporting ocular drugs and nutrients. Although oral medication may have a certain chance of reaching the retina via systemic circulation, it has been difficult to achieve an effective pharmacological effective dose at the target tissue. Therefore, high dose, non-invasive retinal drug delivery remains the great unmet medical need and the most desirable way of ocular drug delivery.

Being able to administer compounds via a periorbital route of administration provides several advantages over other types of administration. Self-administration by the patient is possible. This is in marked contrast to retinal implants, retinal injections, and photocoagulation procedures. These remedial interventions must all be performed by a physician in a medical facility. The side effects of the invasive intravitreal injection (endophthalmitis, retinal detachment, and traumatic cataract) would be completely avoided.

Administration to the periorbital skin is also advantageous compared to eye-drops in many respects. These advantages include the avoidance of reflex blinking and discomfort associated with eye-drops. Since the skin is more rugged and tolerant to exogenously administered substances than the highly sensitive cornea, greater amounts of drug can be administered more frequently, and in preservative-free formulations. Additionally, the eye is much more sensitive to irritation than the surrounding skin. Periorbital delivery thus enables much higher dose strength delivery, as high concentrations of active ingredients in eye drops often cause eye irritation that is not observed on the skin.

Specifically, the studies reported herein reveal that an extremely high concentration of a small molecule in the retina following the application of an “average” quantity (˜160 micrograms) of 4,5-dihydro-N-[4-[[4-(1-methylethoxy) phenyl] methyl] phenyl]-1H-imadazol-2-amine (table 1). Consideration of the data reported in table 1, reveals that administration to the periorbital skin allows extremely high drug concentrations to be well-maintained, at least over a one-day period. An estimate of mass balance is consistent with drug application being mostly confined to the recipient eye; this is in marked contrast to eye drops where only a small percentage of drug is delivered to the interior of the globe. Taken together, it is proposed that administration to the periorbital skin will allow molecules to reach targets embedded in the “hard to reach” neuronal elements in the retina. Underlying the periorbital skin, the or a serrata appears to present no significant barrier to molecules and particulate matter.

### Topical Ophthalmic Administration to the Ocular Surface

In some embodiments, 4,5-dihydro-N-[4-[[4-(1-methylethoxy) phenyl] methyl]phenyl]-1H-imadazol-2-amine is administered via topical ophthalmic administration to the surface of the eye. Administration to the ocular surface has several distinct advantages over other forms of administration that deliver therapeutic agents to the retina and vitreous humor; these advantages include the ability to self-administer, the ease of self-administration, the rapid delivery of compounds to the ocular surface, and the ability to quickly achieve high concentrations at the ocular surface. In some embodiments, administration to the ocular surface also provides for sufficient biodistribution to structures at the posterior of the eye, such as the retina. In some embodiments provided herein, the 4,5-dihydro-N-[4-[[4-(1-methylethoxy) phenyl] methyl] phenyl]-1H-imadazol-2-amine is administered topically to the surface of the eye, such as in an eye drop formulation. In some embodiments, a composition of 4,5-dihydro-N-[4-[[4-(1-methylethoxy) phenyl] methyl] phenyl]-1H-imadazol-2-amine is formulated for topical ophthalmic administration to the ocular surface of the eye as an aqueous solution, a non-aqueous solution, an oil solution, an oil, a gel, a hydrogel, a lotion, an ointment, a dispersion, an emulsion, a cream, or a suspension.

### Formulations

### Compositions for Periorbital Skin Administration

Provided herein in some embodiments are compositions suitable for application to the periorbital skin region of the eye of a subject. In some embodiments, these compositions may be administered through a non-invasive ocular delivery platform (NIODP).

In some embodiments, the composition is in the form of an aqueous solution, a non-aqueous solution, an oil solution, an oil, a gel, a hydrogel, a lotion, an ointment, a dispersion, an emulsion, a cream, and a suspension. In some embodiments, the composition is in the form of an ointment, a cream, or a lotion. In some embodiments, the composition is in the form of an ointment. In some embodiments, the composition is in the form of an aqueous solution. In some embodiments, the composition is in the form of a non-aqueous solution. In some embodiments, the composition is in the form of an oil solution. In some embodiments, the composition is in the form of an oil. In some embodiments, the composition is in the form of a gel. In some embodiments, the composition is in the form of a hydrogel. In some embodiments, the composition is in the form of a lotion. In some embodiments, the composition is in the form of an ointment. In some embodiments, the composition is in the form of a dispersion. In some embodiments, the composition is in the form of an emulsion. In some embodiments, the composition is in the form of a cream. In some embodiments, the composition is in the form of a suspension.

In some embodiments, the composition comprises a semi-solid oleaginous base material. In some embodiments, the composition comprises a petroleum base, a mineral oil, a polyol, a triglyceride, or any combination thereof. In some embodiments, the composition comprises a petroleum base. In some embodiments, the composition comprises petrolatum. In some embodiments, the composition comprises petrolatum, a triglyceride, or any combination thereof. In some embodiments, the composition comprises petrolatum and a triglyceride. In some embodiments, the composition comprises petrolatum, beeswax, or cocoa butter. In some embodiments, the composition comprises beeswax. In some embodiments, the composition comprises cocoa butter.

In some embodiments, the composition comprises an oil. In some embodiments, the composition comprises an oil or a mixture of oils. In some embodiments, the composition comprises a compound provided herein (e.g., an omega-3 fatty acid or 4,5-dihydro-N-[4-[[4-(1-methylethoxy) phenyl] methyl] phenyl]-1H-imadazol-2-amine) mixed in one or more oils. In some embodiments, the composition comprises one or more compounds provided herein (e.g., an omega-3 fatty acid and 4,5-dihydro-N-[4-[[4-(1-methylethoxy) phenyl] methyl] phenyl]-1H-imadazol-2-amine) mixed in one or more oils. In some embodiments, the composition comprises one or more oils proceeded or derived from plants, plant seeds, or nuts. In some embodiments, the plant, plant seed, or nut is coconut, palm kernel, soybean, sesame, olive, vegetable, sunflower, or other plant source, or any combination thereof.

In some embodiments, the composition is mostly an oil. In some embodiments, the composition comprises an oil in an amount of at least about 90%, at least about 91%, at least about 92%, at least about 93%, at least about 94%, at least about 95%, at least about 96%, at least about 97%, at least about 98%, at least about 99%, at least about 99.5%, at least about 99.6%, at least about 99.7%, at least about 99.8%, at least about 99.9%, at least about 99.95%, at least about 99.96%, at least about 99.97%, at least about 99.98%, or at least about 99.99% (w/w) of the composition. In some embodiments, the composition consists essentially of the oil and the active ingredient (e.g., 4,5-dihydro-N-[4-[[4-(1-methylethoxy) phenyl] methyl] phenyl]-1H-imadazol-2-amine).

In some embodiments, the composition comprises the oil in an amount of about 1% to about 100% (w/w) of the composition. In some embodiments, the composition comprises the oil in an amount of about 1% to about 20%, about 1% to about 40%, about 1% to about 60%, about 1% to about 80%, about 1% to about 100%, about 20% to about 40%, about 20% to about 60%, about 20% to about 80%, about 20% to about 100%, about 40% to about 60%, about 40% to about 80%, about 40% to about 100%, about 60% to about 80%, about 60% to about 100%, or about 80% to about 100% (w/w) of the composition. In some embodiments, the composition comprises the oil in an amount of about 1%, about 20%, about 40%, about 60%, about 80%, or about 100% (w/w) of the composition. In some embodiments, the composition comprises the oil in an amount of at least about 1%, about 20%, about 40%, about 60%, or about 80% (w/w) of the composition. In some embodiments, the composition comprises the oil in an amount of at most about 20%, about 40%, about 60%, about 80%, or about 100% (w/w) of the composition.

In some embodiments, the composition comprises a triglyceride. In some embodiments, the triglyceride is a medium-chain or a long-chain triglyceride. In some embodiments, the triglyceride is derived from a natural source. In some embodiments, the triglyceride is derived from plants, plant seeds, or nuts. In some embodiments, the plant, plant seed, or nut comprises a part of a coconut, palm kernel, soybean, a sesame seed or plant, an olive, a sunflower seed or plant, or other vegetable or plant source, or any combination thereof.

In some embodiments, the composition is mostly a triglyceride. In some embodiments, the composition comprises a triglyceride in an amount of at least about 90%, at least about 91%, at least about 92%, at least about 93%, at least about 94%, at least about 95%, at least about 96%, at least about 97%, at least about 98%, at least about 99%, at least about 99.5%, at least about 99.6%, at least about 99.7%, at least about 99.8%, at least about 99.9%, at least about 99.95%, at least about 99.96%, at least about 99.97%, at least about 99.98%, or at least about 99.99% (w/w) of the composition. In some embodiments, the composition consists essentially of the triglyceride and the active ingredient (e.g., 4,5-dihydro-N-[4-[[4-(1-methylethoxy) phenyl] methyl] phenyl]-1H-imadazol-2-amine).

In some embodiments, the composition comprises the triglyceride in an amount of about 1% to about 100% (w/w) of the composition. In some embodiments, the composition comprises the medium-chain triglyceride in an amount of about 1% to about 20%, about 1% to about 40%, about 1% to about 60%, about 1% to about 80%, about 1% to about 100%, about 20% to about 40%, about 20% to about 60%, about 20% to about 80%, about 20% to about 100%, about 40% to about 60%, about 40% to about 80%, about 40% to about 100%, about 60% to about 80%, about 60% to about 100%, or about 80% to about 100% (w/w) of the composition. In some embodiments, the composition comprises the triglyceride in an amount of about 1%, about 20%, about 40%, about 60%, about 80%, or about 100% (w/w) of the composition. In some embodiments, the composition comprises the triglyceride in an amount of at least about 1%, about 20%, about 40%, about 60%, or about 80% (w/w) of the composition. In some embodiments, the composition comprises the triglyceride in an amount of at most about 20%, about 40%, about 60%, about 80%, or about 100% (w/w) of the composition.

In some embodiments, the triglyceride is a medium-chain triglyceride. In some embodiments, the medium-chain triglyceride comprises 2 or 3 medium length fatty acids. In some embodiments, the medium-chain triglyceride comprises C6 or larger fatty acids. In some embodiments, the medium chain triglyceride comprises C6 to C12 fatty acids. In some embodiments, the medium-chain triglyceride comprises a mixture of C6 to C12 fatty acids. In some embodiments, the medium-chain triglyceride comprises fatty acids selected from C6, C8, C10, and C12 fatty acids, or a mixture thereof. In some embodiments, the medium-chain triglyceride comprises caproic acid, caprylic acid, capric acid, lauric acid, or any combination thereof. In some embodiments, the medium-chain triglyceride comprises caprylic acid, capric acid, or a combination thereof. In some embodiments, the medium-chain triglyceride comprises caprylic acid and capric acid. In some embodiments, the medium-chain triglyceride comprises caprylic acid and capric acid in a ratio of about 4:1 (w/w), about 4:3 (w/w), about 3:1 (w/w), about 3:2 (w/w), about 1:1 (w/w), about 2:3 (w/w), about 1:3 (w/w), about 3:4 (w/w), or about 1:4 (w/w). In some embodiments, the ratio is from about 1:1 (w/w) to about 4:1 (w/w). In some embodiments, the ratio is about 3:2 (w/w). In some embodiments, the medium-chain triglyceride comprises at least 75%, at least 80%, at least 85%, at least 90%, at least 910%, at least 92%, at least 93%, at least 94%, or at least 95% C6 to C12 fatty acids as compared to other fatty acids (w/w).

In some embodiments, the composition is mostly a medium chain triglyceride. In some embodiments, the composition comprises a medium-chain triglyceride in an amount of at least about 90%, at least about 91%, at least about 92%, at least about 93%, at least about 94%, at least about 95%, at least about 96%, at least about 97%, at least about 98%, at least about 99%, at least about 99.5%, at least about 99.6%, at least about 99.7%, at least about 99.8%, at least about 99.9%, at least about 99.95%, at least about 99.96%, at least about 99.97%, at least about 99.98%, or at least about 99.99% (w/w) of the composition. In some embodiments, the composition consists essentially of the medium-chain triglyceride and the active ingredient (e.g., 4,5-dihydro-N-[4-[[4-(1-methylethoxy) phenyl] methyl] phenyl]-1H-imadazol-2-amine).

In some embodiments, the composition comprises the medium-chain triglyceride in an amount of about 1% to about 100% (w/w) of the composition. In some embodiments, the composition comprises the medium-chain triglyceride in an amount of about 1% to about 20%, about 1% to about 40%, about 1% to about 60%, about 1% to about 80%, about 1% to about 100%, about 20% to about 40%, about 20% to about 60%, about 20% to about 800%, about 20% to about 100%, about 40% to about 60%, about 40% to about 80%, about 40% to about 100%, about 60% to about 80%, about 60% to about 100%, or about 80% to about 100% (w/w) of the composition. In some embodiments, the composition comprises the medium-chain triglyceride in an amount of about 1%, about 20%, about 40%, about 60%, about 80%, or about 100% (w/w) of the composition. In some embodiments, the composition comprises the medium-chain triglyceride in an amount of at least about 1%, about 20%, about 40%, about 60%, or about 80% (w/w) of the composition. In some embodiments, the composition comprises the medium-chain triglyceride in an amount of at most about 20%, about 40%, about 60%, about 80%, or about 100% (w/w) of the composition.

In some embodiments, the medium-chain triglyceride is derived from a natural source. In some embodiments, the medium-chain triglyceride is derived from coconut, palm, or palm kernel, or combinations thereof. In some embodiments, the medium-chain triglyceride is derived from coconut, or palm. In some embodiments, the medium-chain triglyceride is the oil extracted from the endosperm of coconut or palm. In some embodiments, the medium-chain triglyceride is National Food (NF) grade (NF) or US Pharmacopeia (USP) grade.

In some embodiments, the composition comprises a mixture of petrolatum and a medium-chain triglyceride. In some embodiments, the ratio of petrolatum to medium-chain triglyceride is from about 10:1 (v/v) to about 1:2 (v/v). In some embodiments, the ratio of petrolatum to medium-chain triglyceride is from about 6:1 (v/v) to about 1:1 (v/v). In some embodiments, the ratio of petrolatum to medium-chain triglyceride is from about 6:1 (v/v) to about 1:1 (v/v), from about 5:1 (v/v) to about 1:1 (v/v), from about 4:1 (v/v) to about 1:1 (v/v), from about 3:1 (v/v) to about 2:1 (v/v), or from about 3:2 (v/v) to about 1:1 (v/v). In some embodiments, the ratio of petrolatum to medium-chain triglyceride is about 1:1 (v/v), about 2:1 (v/v), about 3:1 (v/v), about 4:1 (v/v), about 5:1 (v/v), or about 6:1 (v/v). In some embodiments, the ratio of petrolatum to medium-chain triglyceride is about 1:1 (v/v). In some embodiments, the ratio of petrolatum to medium-chain triglyceride is about 2:1 (v/v). In some embodiments, the ratio of petrolatum to medium-chain triglyceride is about 4:1 (v/v).

In some embodiments, the composition further comprises an emollient. In some embodiments, the emollient is selected from a group consisting of vegetable oils, mineral oils, essential oils, essential fatty acids, fatty acids, fatty acid esters, and fatty acid alcohols.

In some embodiments, the composition further comprises a humectant. In some embodiments, the humectant is selected from a group consisting of propylene glycol, aloe vera, lactic acid, glyceryl triacetate, lithium chloride, polydextrose, quillaia, sodium hexametaphosphate, glycerol, sorbitol, xylitol, maltitol, and castor oil.

In some embodiments, the composition further comprises a thickening agent. In some embodiments, the thickening agent is selected from a group consisting of fatty acids, fatty acid esters, and fatty acid alcohols.

In some embodiments, the composition further comprises a preservative. In some embodiments, the preservative is selected from a group consisting of sodium borate/boric acid, polyhexamthethylene biguanide (phmb), parabens (parahydroxy benzoic acid derivatives; phenyl mercuric nitrate, benzalkonium chloride, benzelthonium chloride, chlorhexidine, chlorbutanol, methyl paraben, phenylethyl alcohol, phenylmercuric acetate, phenylmercuric nitrate, propyl paraben, and thimerosal.

In some embodiments, the composition is free from preservatives. In some embodiments, the composition is free from benzalkonium chloride.

In some embodiments, the composition further comprises an antimicrobial. In some embodiments, the antimicrobial is selected from a group consisting of basil, oregano, thyme, citrus oils and monoterpene, sesquiterpenes, and phenylpropanoids.

In some embodiments, the composition further comprises a penetration enhancer. In some embodiments, the penetration enhancer is selected from a group consisting of ethanol, isopropyl alcohol, d-hexanol, octanol, doctanol, myristyl alcohol, ethyl acetate, oleoyl acetate, isopropyl myristate, azone, carbamide, glycerylmono-oleate, octyl salicylate, propylene glycol, dipropylene glycol, 1,2-butylene glycol, oleic acid, N-methyl-2-pyrrolidone, 2-pyrrolidone, 2-pyrrolidone-5-carboxylic acid, dimethyl sulfoxide, decylmethyl sulfoxide, sodium lauryl sulfate, Span 80, Tween 80, cineole, eugenol, D-limonene, menthol, menthane, cyclodextrins, hyaluronic acid, and vitamin E.

In some embodiments, the composition further comprises an odor masking agent. Odor masking agents are especially suitable for compositions which comprise a component derived from the tissue of an animal (e.g., omega-3 fatty acids derived from fish) which may carry a residual odor. In some embodiments, the odor masking agent is an essential oil (e.g., a floral, fruit, wood, mint, herbal, or other essential oil).

### Compositions of JV-DE1 for Periorbital Skin Administration

In some embodiments, the compositions provided herein suitable for periorbital skin administration comprise 4, 5-dihydro-N-[4-[[4-(1-methylethoxy) phenyl] methyl] phenyl]-1H-imadazol-2-amine as an active ingredient. The composition comprising 4, 5-dihydro-N-[4-[[4-(1-methylethoxy) phenyl] methyl] phenyl]-1H-imadazol-2-amine may be any of the compositions provided herein. In some embodiments, the composition comprises a medium-chain triglyceride.

In some embodiments, the 4,5-dihydro-N-[4-[[4-(1-methylethoxy) phenyl]methyl] phenyl]-1H-imadazol-2-amine is present in an amount of about 0.00005% to about 10% (w/w) of the composition. In some embodiments, the 4,5-dihydro-N-[4-[[4-(1-methylethoxy) phenyl] methyl] phenyl]-1H-imadazol-2-amine is present in an amount of about 0.00005% to about 0.0005%, about 0.00005% to about 0.005%, about 0.00005% to about 0.05%, about 0.00005% to about 0.5%, about 0.00005% to about 1%, about 0.00005% to about 10%, about 0.0005% to about 0.005%, about 0.0005% to about 0.05%, about 0.0005% to about 0.5%, about 0.0005% to about 1%, about 0.0005% to about 10%, about 0.005% to about 0.05%, about 0.005% to about 0.5%, about 0.005% to about 1%, about 0.005% to about 10%, about 0.05% to about 0.5%, about 0.05% to about 1%, about 0.05% to about 10%, about 0.5% to about 1%, about 0.5% to about 10%, or about 1% to about 10% (w/w) of the composition. In some embodiments, the 4,5-dihydro-N-[4-[[4-(1-methylethoxy) phenyl] methyl] phenyl]-1H-imadazol-2-amine is present in an amount of about 0.005%, about 0.01%, about 0.02%, about 0.03%, about 0.040%, about 0.05%, about 0.06%, about 0.07%, about 0.08%, about 0.09%, or about 0.1% (w/w) of the composition. In some embodiments, the 4,5-dihydro-N-[4-[[4-(1-methylethoxy) phenyl] methyl] phenyl]-1H-imadazol-2-amine is present in an amount of about 0.005% to about 0.3% (w/w) of the composition. In some embodiments, the 4,5-dihydro-N-[4-[[4-(1-methylethoxy) phenyl] methyl] phenyl]-1H-imadazol-2-amine is present in an amount of about 0.005% to about 0.01%, about 0.005% to about 0.03%, about 0.005% to about 0.06%, about 0.005% to about 0.1%, about 0.005% to about 0.3%, about 0.01% to about 0.03%, about 0.01% to about 0.06%, about 0.01% to about 0.1%, about 0.01% to about 0.3%, about 0.03% to about 0.06%, about 0.03% to about 0.1%, about 0.03% to about 0.3%, about 0.06% to about 0.1%, about 0.06% to about 0.3%, or about 0.1% to about 0.3% (w/w) of the composition.

In some embodiments, the compositions provided herein are configured to dispense a set amount of 4,5-dihydro-N-[4-[[4-(1-methylethoxy) phenyl] methyl] phenyl]-1H-imadazol-2-amine per administration. In some embodiments, the composition is configured to dispense from about 10 ng to about 5 mg of the 4,5-dihydro-N-[4-[[4-(1-methylethoxy) phenyl]methyl] phenyl]-1H-imadazol-2-amine per administration. In some embodiments, the composition is configured to dispense about 1 microgram to about 500 micrograms of the 4,5-dihydro-N-[4-[[4-(1-methylethoxy) phenyl] methyl] phenyl]-1H-imadazol-2-amine per administration. In some embodiments, the composition is configured to dispense about 1 microgram to about 5 micrograms, about 1 microgram to about 10 micrograms, about 1 microgram to about 25 micrograms, about 1 microgram to about 50 micrograms, about 1 microgram to about 75 micrograms, about 1 microgram to about 100 micrograms, about 1 microgram to about 200 micrograms, about 1 microgram to about 300 micrograms, about 1 microgram to about 400 micrograms, about 1 microgram to about 500 micrograms, about 5 micrograms to about 10 micrograms, about 5 micrograms to about 25 micrograms, about 5 micrograms to about 50 micrograms, about 5 micrograms to about 75 micrograms, about 5 micrograms to about 100 micrograms, about 5 to about 200 micrograms, about 5 to about 300 micrograms, about 5 to about 400 micrograms, about 5 to about 500 micrograms, about 10 micrograms to about 25 micrograms, about 10 micrograms to about 50 micrograms, about 10 micrograms to about 75 micrograms, about 10 micrograms to about 100 micrograms, about 10 to about 200 micrograms, about 10 to about 300 micrograms, about 10 to about 400 micrograms, about 10 to about 500 micrograms, about 25 micrograms to about 50 micrograms, about 25 micrograms to about 75 micrograms, about 25 micrograms to about 100 micrograms, about 25 to about 200 micrograms, about 25 to about 300 micrograms, about 25 to about 400 micrograms, about 25 to about 500 micrograms, about 50 micrograms to about 75 micrograms, about 50 micrograms to about 100 micrograms, about 50 to about 200 micrograms, about 50 to about 300 micrograms, about 5 to about 400 micrograms, about 50 to about 500 micrograms, about 75 micrograms to about 100 micrograms, about 75 to about 200 micrograms, about 75 to about 300 micrograms, about 75 to about 400 micrograms, about 75 to about 500 micrograms, about 100 to about 200 micrograms, about 100 to about 300 micrograms, about 100 to about 400 micrograms, about 100 to about 500 micrograms, about 200 to about 500 micrograms, about 300 to about 500 micrograms, or about 400 to about 500 micrograms of the 4,5-dihydro-N-[4-[[4-(1-methylethoxy) phenyl] methyl] phenyl]-1H-imadazol-2-amine per administration.

### Compositions of Omega-3 Fatty Acids for External Eyelid or Periorbital Skin Administration

In some embodiments, the compositions suitable for topical periorbital skin administration provided herein comprise an omega-3 fatty acid, or a pharmaceutically acceptable ester or salt thereof, and a pharmaceutically acceptable excipient. In some embodiments, these compositions may be administered through a non-invasive ocular delivery platform (NIODP).

In some embodiments, the compositions suitable for topical external eyelid skin administration provided herein comprise an omega-3 fatty acid, or a pharmaceutically acceptable ester or salt thereof, and a pharmaceutically acceptable excipient.

In some embodiments, the omega-3 fatty acid is present in the composition of from about 0.01% to about 100% (w/w) of the composition. In some embodiments, the omega-3 fatty acid is present in an amount of about 0.01% to about 50% (w/w) of the composition. In some embodiments, the omega-3 fatty acid is present in an amount of about 1% to about 50% (w/w) of the composition. In some embodiments, the omega-3 fatty acid is present in an amount of about 1% to about 5%, about 1% to about 10%, about 1% to about 20%, about 1% to about 30%, about 1% to about 40%, about 1% to about 50%, about 5% to about 10%, about 5% to about 20%, about 5% to about 30%, about 5% to about 40%, about 5% to about 50%, about 5% to about 75%, about 5% to about 100%, about 10% to about 20%, about 10% to about 30%, about 10% to about 40%, about 10% to about 50%, about 10% to about 75%, about 10% to about 100%, about 20% to about 30%, about 20% to about 40%, about 20% to about 50%, about 20% to about 75%, about 20% to about 100%, about 30% to about 40%, about 30% to about 50%, about 30% to about 75%, about 30% to about 100%, about 40% to about 50%, about 40% to about 75%, about 40% to about 100%, about 50% to about 75%, about 50% to about 100%, or about 75% to about 100%, (w/w) of the composition. In some embodiments, the omega-3 fatty acid is present in an amount of up to about 20%, about 30%, about 40%, about 50%, about 60%, about 70%, about 80%, about 90% or about 100% (w/w) of the composition. In some embodiments, the omega-3 fatty acid is present in an amount of about 10% to about 15%, about 10% to about 20%, about 10% to about 25%, about 10% to about 30%, about 15% to about 20%, about 15% to about 25%, about 15% to about 30%, about 20% to about 25%, about 20% to about 30%, or about 25% to about 30% (w/w) of the composition. In some embodiments, the omega-3 fatty acid is present in an amount of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (w/w) of the composition. In some embodiments, the omega-3 fatty acid is administered alone (e.g., without any vehicle).

In some embodiments, the composition is configured to deliver the omega-3 fatty acid in an amount of about 0.1 mg to about 3000 mg, about 0.1 mg to about 1000 mg, about 0.1 mg to about 500 mg, about 0.1 mg to about 300 mg, about 0.1 mg to about 200 mg, about 0.1 mg to about 100 mg. In some embodiments, the composition is configured to deliver the omega-3 fatty acid in an amount of about 0.1 mg to about 100 mg. In some embodiments, the composition is configured to deliver the omega-3 fatty acid in an amount of about 0.1 mg to about 1 mg, about 0.1 mg to about 10 mg, about 0.1 mg to about 20 mg, about 0.1 mg to about 50 mg, about 0.1 mg to about 100 mg, about 1 mg to about 10 mg, about 1 mg to about 20 mg, about 1 mg to about 50 mg, about 1 mg to about 100 mg, about 10 mg to about 20 mg, about 10 mg to about 50 mg, about 10 mg to about 100 mg, about 20 mg to about 50 mg, about 20 mg to about 100 mg, or about 50 mg to about 100 mg. In some embodiments, the composition is configured to deliver the omega-3 fatty acid in an amount of about 0.1 mg, about 1 mg, about 10 mg, about 20 mg, about 50 mg, or about 100 mg. In some embodiments, the composition is configured to deliver the omega-3 fatty acid in an amount of at least about 0.1 mg, about 1 mg, about 10 mg, about 20 mg, or about 50 mg. In some embodiments, the composition is configured to deliver the omega-3 fatty acid in an amount of at most about 1 mg, about 10 mg, about 20 mg, about 50 mg, or about 100 mg.

In some embodiments, the composition comprises a vehicle for the delivery of the omega-3 fatty acid. In some embodiments, the vehicle comprises an oil. In some embodiments, the vehicle comprises an oil or a mixture of oils. In some embodiments, the vehicle comprises an omega-3 fatty acid dissolved in one or more oils. In some embodiments, the oil is derived from a natural source. In some embodiments, the vehicle comprises one or more oils derived from plants, plant seeds, or nuts. In some embodiments, the plant, plant seed, or nut is soybean, sesame, olive, vegetable, sunflower, or other plant source, or any combination thereof.

In some embodiments, the vehicle is an oil. In some embodiments, the composition comprises the oil in an amount of about 1% to about 100% (w/w) of the composition. In some embodiments, the composition comprises the oil in an amount of about 1% to about 20%, about 1% to about 40%, about 1% to about 60%, about 1% to about 80%, about 1% to about 100%, about 20% to about 40%, about 20% to about 60%, about 20% to about 80%, about 20% to about 100%, about 40% to about 60%, about 40% to about 80%, about 40% to about 100%, about 60% to about 80%, about 60% to about 100%, or about 80% to about 100% (w/w) of the composition. In some embodiments, the composition comprises the oil in an amount of about 1%, about 20%, about 40%, about 60%, about 80%, or about 100% (w/w) of the composition. In some embodiments, the composition comprises the oil in an amount of at least about 1%, about 20%, about 40%, about 60%, or about 80% (w/w) of the composition. In some embodiments, the composition comprises the oil in an amount of at most about 20%, about 40%, about 60%, about 80%, or about 100% (w/w) of the composition. In some embodiments, the oil comprises from about 50% to about 99% (w/w) of the composition. In some embodiments, the oil comprises about 50% to about 60%, about 50% to about 70%, about 50% to about 80%, about 50% to about 90%, about 50% to about 99%, about 60% to about 70%, about 60% to about 80%, ab out 60% to about 90%, about 60% to about 99%, about 70% to about 80%, about 70% to about 90%, about 70% to about 99%, about 80% to about 90%, about 80% to about 99%, or about 90% to about 99% (w/w) of the composition. In some embodiments, the oil comprises about 50%, about 60%, about 70%, about 80%, about 90%, or about 99% (w/w) of the composition.

In some embodiments, the vehicle comprises a triglyceride. In some embodiments, the triglyceride is a medium-chain or a long-chain triglyceride. In some embodiments, the triglyceride is derived from a natural source. In some embodiments, the triglyceride is derived from plants, plant seeds, or nuts. In some embodiments, the plant, plant seed, or nut comprises a part of a coconut, palm kernel, soybean, a sesame seed or plant, an olive, a sunflower seed or plant, or other vegetable or plant source, or any combination thereof.

In some embodiments, the composition comprises the triglyceride in an amount of about 1% to about 100% (w/w) of the composition. In some embodiments, the composition comprises the medium-chain triglyceride in an amount of about 1% to about 20%, about 1% to about 40%, about 1% to about 60%, about 1% to about 80%, about 1% to about 100%, about 20% to about 40%, about 20% to about 60%, about 20% to about 80%, about 20% to about 100%, about 40% to about 60%, about 40% to about 80%, about 40% to about 100%, about 60% to about 80%, about 60% to about 100%, or about 80% to about 100% (w/w) of the composition. In some embodiments, the composition comprises the triglyceride in an amount of about 1%, about 20%, about 40%, about 60%, about 80%, or about 100% (w/w) of the composition. In some embodiments, the composition comprises the triglyceride in an amount of at least about 1%, about 20%, about 40%, about 60%, or about 80% (w/w) of the composition. In some embodiments, the composition comprises the triglyceride in an amount of at most about 20%, about 40%, about 60%, about 80%, or about 100% (w/w) of the composition. In some embodiments, the triglyceride comprises from about 50% to about 99% (w/w) of the composition. In some embodiments, the triglyceride comprises about 50% to about 60%, about 50% to about 70%, about 50% to about 80%, about 50% to about 90%, about 50% to about 99%, about 60% to about 70%, about 60% to about 80%, about 60% to about 90%, about 60% to about 99%, about 70% to about 80%, about 70% to about 90%, about 70% to about 99%, about 80% to about 90%, about 80% to about 99%, or about 90% to about 99% (w/w) of the composition. In some embodiments, the triglyceride comprises about 50%, about 60%, about 70%, about 80%, about 90%, or about 99% (w/w) of the composition.

In some embodiments, the triglyceride is a medium-chain triglyceride. In some embodiments, the medium-chain triglyceride comprises 2 or 3 medium length fatty acids. In some embodiments, the medium-chain triglyceride comprises C6 or larger fatty acids. In some embodiments, the medium chain triglyceride comprises C6 to C12 fatty acids. In some embodiments, the medium-chain triglyceride comprises a mixture of C6 to C12 fatty acids. In some embodiments, the medium-chain triglyceride comprises fatty acids selected from C6, C8, C10, and C12 fatty acids, or a mixture thereof. In some embodiments, the medium-chain triglyceride comprises caproic acid, caprylic acid, capric acid, lauric acid, or any combination thereof. In some embodiments, the medium-chain triglyceride comprises caprylic acid, capric acid, or a combination thereof. In some embodiments, the medium-chain triglyceride comprises caprylic acid and capric acid. In some embodiments, the medium-chain triglyceride comprises caprylic acid and capric acid in a ratio of about 4:1 (w/w), about 4:3 (w/w), about 3:1 (w/w), about 3:2 (w/w), about 1:1 (w/w), about 2:3 (w/w), about 1:3 (w/w), about 3:4 (w/w), or about 1:4 (w/w). In some embodiments, the ratio is from about 1:1 (w/w) to about 4:1 (w/w). In some embodiments, the ratio is about 3:2 (w/w). In some embodiments, the medium-chain triglyceride comprises at least 75%, at least 80%, at least 85%, at least 90%, at least 91%, at least 92%, at least 93%, at least 94%, or at least 95% C6 to C12 fatty acids as compared to other fatty acids (w/w).

In some embodiments, the composition comprises the medium-chain triglyceride in an amount of about 1% to about 100% (w/w) of the composition. In some embodiments, the composition comprises the medium-chain triglyceride in an amount of about 1% to about 20%, about 1% to about 40%, about 1% to about 60%, about 1% to about 80%, about 1% to about 100%, about 20% to about 40%, about 20% to about 60%, about 20% to about 800%, about 20% to about 100%, about 40% to about 60%, about 40% to about 80%, about 40% to about 100%, about 60% to about 80%, about 60% to about 100%, or about 80% to about 100% (w/w) of the composition. In some embodiments, the composition comprises the medium-chain triglyceride in an amount of about 1%, about 20%, about 40%, about 60%, about 80%, or about 100% (w/w) of the composition. In some embodiments, the composition comprises the medium-chain triglyceride in an amount of at least about 1%, about 20%, about 40%, about 60%, or about 80% (w/w) of the composition. In some embodiments, the composition comprises the medium-chain triglyceride in an amount of at most about 20%, about 40%, about 60%, about 80%, or about 100% (w/w) of the composition. In some embodiments, the medium-chain triglyceride comprises from about 50% to about 99% (w/w) of the composition. In some embodiments, the medium-chain triglyceride comprises about 50% to about 60%, about 50% to about 70%, about 50% to about 80%, about 50% to about 90%, about 50% to about 99%, about 60% to about 70%, about 60% to about 80%, about 60% to about 90%, about 60% to about 99%, about 70% to about 80%, about 70% to about 90%, about 70% to about 99%, about 80% to about 90%, about 80% to about 99%, or about 90% to about 99% (w/w) of the composition. In some embodiments, the medium-chain triglyceride comprises about 50%, about 60%, about 70%, about 80%, about 90%, or about 99% (w/w) of the composition.

In some embodiments, the medium-chain triglyceride is derived from a natural source. In some embodiments, the medium-chain triglyceride is derived from coconut, palm, or palm kernel, or combinations thereof. In some embodiments, the medium-chain triglyceride is derived from coconut, or palm. In some embodiments, the medium-chain triglyceride is the oil extracted from the endosperm of coconut or palm. In some embodiments, the medium-chain triglyceride is National Food (NF) grade (NF) or US Pharmacopeia (USP) grade.

In some embodiments, the vehicle is a fatty acid vehicle. In some embodiments, the fatty acid vehicle is an unsaturated fatty acid. In some embodiments, the fatty acid vehicle is a C14 to C22 fatty acid. In some embodiments, the fatty acid vehicle is a C14 to C22 unsaturated fatty acid. In some embodiments, the fatty acid vehicle comprises linoleic acid.

In some embodiments, the fatty acid vehicle comprises from about 1% to about 100% (w/w) of the composition. In some embodiments, the fatty acid vehicle comprises from about 1% to about 20%, about 1% to about 40%, about 1% to about 60%, about 1% to about 80%, about 1% to about 100%, about 20% to about 40%, about 20% to about 60%, about 20% to about 80%, about 20% to about 100%, about 40% to about 60%, about 40% to about 80%, about 40% to about 100%, about 60% to about 80%, about 60% to about 100%, or about 80% to about 100% (w/w) of the composition. In some embodiments, the fatty acid vehicle comprises from about 1%, about 20%, about 40%, about 60%, about 80%, or about 100% (w/w) of the composition. In some embodiments, the fatty acid vehicle comprises from at least about 1%, about 20%, about 40%, about 60%, or about 80% (w/w) of the composition. In some embodiments, the fatty acid vehicle comprises from at most about 20%, about 40%, about 60%, about 80%, or about 100% (w/w) of the composition. In some embodiments, the fatty acid vehicle comprises from about 50% to about 99% (w/w) of the composition. In some embodiments, the fatty acid vehicle comprises about 50% to about 60%, about 50% to about 70%, about 50% to about 80%, about 50% to about 90%, about 50% to about 99%, about 60% to about 70%, about 60% to about 80%, about 60% to about 90%, about 60% to about 99%, about 70% to about 80%, about 70% to about 90%, about 70% to about 99%, about 80% to about 90%, about 80% to about 99%, or about 90% to about 99% (w/w) of the composition. In some embodiments, the fatty acid vehicle comprises about 50%, about 60%, about 70%, about 80%, about 90%, or about 99% (w/w) of the composition.

In some embodiments, administration of the composition to a patient via a non-invasive ocular delivery platform results in a physiologically relevant amount of the omega-3 fatty acid to at least one portion of the eye.

In some embodiments, the portion of the eye is the upper eyelid, the cornea, the retina, or any combination thereof.

In some embodiments, administration of the composition to a patient results in a therapeutically or other beneficially relevant amount of the omega-3 fatty acid to at least one portion of the eye.

In some embodiments, the portion of the eye is the upper eyelid, the cornea, the retina, or any combination thereof.

In some embodiments, administration of the composition to the patient results in a level of the omega-3 fatty acid at least 10 μg/g, at least 50 μg/g, at least 100 μg/g, at least 150 μg/g, at least 200 μg/g, at least 250 μg/g, at least 300 μg/g, at least 350 μg/g at least 400 μg/g, or at least 500 μg/g above baseline levels in the upper eyelid 30 minutes after administration.

In some embodiments, administration of the composition to the patient results in a level of the omega-3 fatty acid at least 10 μg/g, at least 15 μg/g, at least 20 μg/g, at least 25 μg/g, at least 30 μg/g, at least 35 μg/g, at least 40 μg/g, at least 50 μg/g, at least 60 μg/g, or at least 70 μg/g, above baseline levels in the cornea 30 minutes after administration.

In some embodiments, administration of the composition to the patient results in a level of the omega-3 fatty acid at least 10 μg/g, at least 15 μg/g, at least 20 μg/g, at least 25 μg/g, at least 30 μg/g, at least 35 μg/g, at least 40 μg/g, at least 45 μg/g, at least 50 μg/g, at least 60 μg/g, or at least 70 μg/g, at least 80 μg/g, at least 90 μg/g, at least 100 μg/g, at least 110 μg/g, or at least 120 μg/g above baseline levels in the retina 30 minutes after administration.

### Compositions for Topical Ophthalmic Administration of JV-DE1

Also provided herein are compositions of 4,5-dihydro-N-[4-[[4-(1-methylethoxy) phenyl] methyl] phenyl]-1H-imadazol-2-amine (a.k.a JV-DE1) suitable for topical ophthalmic administration. In some embodiments, the topical ophthalmic administration is to the surface of the eye. In some embodiments, the composition is formulated as eye drops. In some embodiments, the composition is formulated as an aqueous solution, a non-aqueous solution, an oil solution, an oil, a gel, a hydrogel, a lotion, an ointment, a dispersion, an emulsion, a cream, in liposomes, or in nanoparticles, or a suspension. In some embodiments, the composition is formulated as an aqueous solution. In some embodiments, the topical ophthalmic administration is to the periorbital skin of a patient.

In some embodiments, the composition comprises a polyoxyl castor oil. In some embodiments, the polyoxyl castor oil is a poly(alkylene oxide) castor oil. In some embodiments, the polyoxyl castor oil comprises poly(alkylene oxide) subunits selected from poly(ethylene glycol) (PEG), poly(propylene glycol), or any combination thereof.

In some embodiments, the polyoxyl castor oil is a PEGylated castor oil. In som e embodiments, the molar ratio of PEG to castor oil in the PEGylated castor oil is in the range of from about 20:1 to about 50:1. In some embodiments, the molar ratio of PEG to castor oil is from about 25:1 to about 45:1, or from about 30:1 to about 40:1. In some embodiments, the molar ratio of PEG to castor oil is about 35:1. In some embodiments, the polyoxyl castor oil is polyoxyl 35 castor oil.

In some embodiments, the polyoxyl castor oil is present in an amount of from about 0.1% to about 20%, 0.1% to about 15%, 0.1% to about 10%, or 0.1% to about 5% (w/w) of the composition. In some embodiments, the polyoxyl castor oil is present in an amount of from about 0.1% to about 5% (w/w) of the composition. In some embodiments, the polyoxyl castor oil is present in an amount of about 0.1% to about 0.5%, about 0.1% to about 1%, about 0.1% to about 2%, about 0.1% to about 5%, about 0.5% to about 1%, about 0.5% to about 2%, about 0.5% to about 5%, about 1% to about 2%, about 1% to about 5%, or about 2% to about 5% (w/w) of the composition. In some embodiments, the polyoxyl castor oil is present in an amount of about 0.5% to about 1.5% (w/w) of the composition. In some embodiments, the polyoxyl castor oil is present in an amount of about 0.5%, about 0.6%, about 0.70%, about 0.8%, about 0.9%, about 1%, about 1.1%, about 1.20%, about 1.3%, about 1.40%, or about 1.5%. In some embodiments, the polyoxyl castor oil is present in an amount of about 0.5% to about 1.5%, about 0.6% to about 1.4%, about 0.7% to about 1.3%, about 0.8% to about 1.2%, or about 0.9% to about 1.1% (w/w) of the composition.

In some embodiments, the polyoxyl 35 castor oil is present in an amount of from about 0.1% to about 20%, 0.1% to about 15%, 0.1% to about 10%, or 0.1% to about 5% (w/w) of the composition. In some embodiments, the polyoxyl 35 castor oil is present in an amount of from about 0.1% to about 5% (w/w) of the composition. In some embodiments, the polyoxyl 35 castor oil is present in an amount of about 0.1% to about 0.5%, about 0.1% to about 1%, about 0.1% to about 2%, about 0.1% to about 5%, about 0.5% to about 1%, about 0.5% to about 2%, about 0.5% to about 5%, about 1% to about 2%, about 1% to about 5%, or about 2% to about 5% (w/w) of the composition. In some embodiments, the polyoxyl 35 castor oil is present in an amount of about 0.5% to about 1.5% (w/w) of the composition. In some embodiments, the polyoxyl 35 castor oil is present in an amount of about 0.5%, about 0.6%, about 0.7%, about 0.8%, about 0.9%, about 1%, about 1.1%, about 1.2%, about 1.30%, about 1.4%, or about 1.5%. In some embodiments, the polyoxyl 35 castor oil is present in an amount of about 0.5% to about 1.5%, about 0.6% to about 1.4%, about 0.7% to about 1.3%, about 0.8% to about 1.2%, or about 0.9% to about 1.1% (w/w) of the composition.

In some embodiments, the composition further comprises an ocular surface lubricating agent. In some embodiments, the ocular surface lubricating agent is selected from polyethylene glycol, propylene glycol, polyvinyl alcohol, castor oil or glycerol. In some embodiments, the ocular surface lubricating agent is glycerol.

In some embodiments, the ocular surface lubricating agent is present in an amount of about 0.05% to about 2% (w/w) of the composition. In some embodiments, the ocular surface lubricating agent is present in an amount of about 0.05% to about 0.5% (w/w) of the composition. In some embodiments, the ocular surface lubricating agent is present in an amount of about 0.05%, about 0.1%, about 0.15%, about 0.2%, about 0.25%, about 0.3%, about 0.4%, or about 0.5% (w/w) of the composition. In some embodiments, the ocular surface lubricating agent is present in an amount of about 0.05% to about 0.1%, about 0.05% to about 0.15%, about 0.05% to about 0.20%, about 0.05% to about 0.25%, about 0.05% to about 0.3%, about 0.05% to about 0.4%, about 0.05% to about 0.5%, about 0.1% to about 0.15%, about 0.1% to about 0.2%, about 0.1% to about 0.25%, about 0.1% to about 0.3%, about 0.1% to about 0.4%, about 0.1% to about 0.5%, about 0.15% to about 0.20%, about 0.15% to about 0.25%, about 0.15% to about 0.3%, about 0.15% to about 0.4%, about 0.15% to about 0.5%, about 0.2% to about 0.25%, about 0.2% to about 0.3%, about 0.2% to about 0.4%, about 0.2% to about 0.5%, about 0.25% to about 0.3%, about 0.25% to about 0.40%, about 0.25% to about 0.5%, about 0.3% to about 0.40%, about 0.3% to about 0.5%, or about 0.4% to about 0.5% (w/w) of the composition.

In some embodiments, the composition further comprises a buffer. In some embodiments, the buffer is selected from triethanolamine (tris), histidine, bicarbonate; N-(2-Hydroxyethyl)piperazine-N′-(2-ethanesulfonic acid) (HEPES); 2-(N-Morpholino)ethanesulfonic acid (MES); 2-(N-Morpholino)ethanesulfonic acid sodium salt (MES); 3-(N-Morpholino)propanesulfonic acid (MOPS); and N-tris[Hydroxymethyl]methyl-3-aminopropanesulfonic acid (TAPS). In some embodiments, the buffer is tris. In some embodiments, the composition comprises tris buffered saline.

In some embodiments, the composition comprises an oil. In some embodiments, the composition comprises an oil or a mixture of oils. In some embodiments, the composition comprises 4,5-dihydro-N-[4-[[4-(1-methylethoxy) phenyl] methyl] phenyl]-1H-imadazol-2-amine mixed in one or more oils. In some embodiments, the composition comprises one or more oils derived from plants, plant seeds, or nuts. In some embodiments, the plant, plant seed, or nut is soybean, sesame, olive, vegetable, sunflower, or other plant source, or any combination thereof. In some embodiments, the oil comprises a triglyceride. In some embodiments, the oil comprises a medium chain triglyceride.

In some embodiments, the composition is mostly an oil. In some embodiments, the composition comprises an oil in an amount of at least about 90%, at least about 910%, at least about 92%, at least about 93%, at least about 94%, at least about 95%, at least about 96%, at least about 97%, at least about 98%, at least about 99%, at least about 99.5%, at least about 99.6%, at least about 99.7%, at least about 99.8%, at least about 99.9%, at least ab out 99.95%, at least about 99.96%, at least about 99.97%, at least about 99.98%, or at least about 99.99% (w/w) of the composition. In some embodiments, the composition consists essentially of the oil and 4,5-dihydro-N-[4-[[4-(1-methylethoxy) phenyl] methyl] phenyl]-1H-imadazol-2-amine.

In some embodiments, the composition comprises the oil in an amount of about 1% to about 100% (w/w) of the composition. In some embodiments, the composition comprises the oil in an amount of about 1% to about 20%, about 1% to about 40%, about 1% to about 60%, about 1% to about 80%, about 1% to about 100%, about 20% to about 40%, about 20% to about 60%, about 20% to about 80%, about 20% to about 100%, about 40% to about 60%, about 40% to about 80%, about 40% to about 100%, about 60% to about 80%, about 60% to about 100%, or about 80% to about 100% (w/w) of the composition. In some embodiments, the composition comprises the oil in an amount of about 1%, about 20%, about 40%, about 60%, about 80%, or about 100% (w/w) of the composition. In some embodiments, the composition comprises the oil in an amount of at least about 1%, about 20%, about 40%, about 60%, or about 80% (w/w) of the composition. In some embodiments, the composition comprises the oil in an amount of at most about 20%, about 40%, about 60%, about 80%, or about 100% (w/w) of the composition. In some embodiments, the composition has a pH of from about 6.5 to about 8.5. In some embodiments, the composition has a pH of from about 6.7 to about 8.3, about 7.0 to about 8.0, about 7.2 to about 7.8, or about 7.3 to about 7.7. In some embodiments, the composition has a pH of about 7.0, 7.1, 7.2, 7.3, 7.4, 7.5, 7.6, 7.7, 7.8, 7.9, or 8.0.

In some embodiments, the composition comprises an omega-3 fatty acid or a pharmaceutically acceptable ester or salt thereof. In some embodiments, the omega-3 fatty acid is isolated from fish tissue. In some embodiments, the omega-3 fatty acid is isolated from a plant source. In some embodiments, the omega-3 fatty acid comprises alpha-linolenic acid (ALA), eicosapentaenoic acid (EPA), docosahexaenoic acid (DHA), or any combination thereof.

### Methods of Treatment

Provided herein are methods of treatment of the disease and disorder provided herein with a compound as provided herein. In some embodiments, the compound is 4,5-dihydro-N-[4-[[4-(1-methylethoxy) phenyl] methyl] phenyl]-1H-imadazol-2-amine. In some embodiments, the compound is an omega-3 fatty acid.

In some embodiments, the compound is administered to each eye of the patient four times per day, three times per day, twice per day, once per day, once every other day, once every three days, once every four days, or once every seven days, or any combination thereof (e.g. a variable dosing protocol). In some embodiments, the compound is administered to each eye of the patient once per week, twice per week, three times per week, once every two weeks, or once every three weeks. In some embodiments, the compound is administered to each eye of the patient once per day.

In some embodiments, the compound is administered to one eye of the patient twice per day, once per day, once every other day, once every three days, once every four days, or once every seven days, or any combination thereof (e.g. a variable dosing protocol). In some embodiments, the compound is administered to one eye of the patient once per week, twice per week, three times per week, once a week, once every two weeks, or once every three weeks. In some embodiments, the compound is administered to one eye of the patient once per day.

In some embodiments, the compound is administered ad libitum with respect to either or both eyes.

In some embodiments, the compound is administered to the ocular surface of the eye. In some embodiments, the compound is administered by dropper, pump, spray, click pen or tube.

In some embodiments, the compound is applied to the periorbital skin using a device. In some embodiments, the device is a dropper, a pump, a spray, a click pen or a tube reservoir device. In some embodiments, the compound is administered topically by brush, Q-tip, or spatula.

In some embodiments, the compound is applied to the periorbital skin using an eye pad. An eye pad, also known as eye patch, is a small (and may be sterile) pad large enough to cover the periorbital region of the eye, specifically designed for absorption of formulation for periorbital or eyelid administration. In some embodiments, the eye pad comprises a preselected dosage of an active ingredient. A subject may apply the eye pad to the periorbital skin for a certain period of time. The time may depend on the desired dose of active ingredient desired. In some embodiments, the eye pad may be applied to the periorbital skin of a patient for 1 minute, 5 minutes, 10 minutes, 20 minutes, 30 minutes, 45 minutes, or 1 hour.

In some embodiments, the device releases a preselected dosage in a uniform manner onto the periorbital skin of the patient. In some embodiments, the compound is applied by a roller device to the periorbital skin. In some embodiments, the compound is applied by a Q-tip to the periorbital skin. In some embodiments, the compound is applied by a spatula to the periorbital skin. In some embodiments, the application process may be preceded by using a graduated dropper, syringe, click pen or pipette.

It may be beneficial to use formulation that does not comprise preservatives. In some embodiments, the compound is packaged in a single-use container. In some embodiments, the single-use container is a blow-fill-seal capsule. In some embodiments, the single-use container is a soft gel capsule. In some embodiments, the compound is packaged in a multi-use container. In some embodiments, the multi-use container is an airless pump or drop bottle. In some embodiments, packaging is designed to minimize the fishy smell that may be caused my oxidation of an omega-3 fatty acid.

In some embodiments, the compound is administered to the periorbital skin above the upper eyelid, below the lower eyelid, or both above the upper and below the lower eyelids. In some embodiments, the compound is administered above the upper eyelid. In some embodiments, the compound is administered below the lower eyelid. In some embodiments, the compound is administered both above the upper and below the lower eyelid.

In some embodiments, penetration through the periorbital skin is increased by a penetration enhancer, tape-stripping, microdermabrasion, solvent, pulsed laser, and iontophoresis, which has been found useful for delivering macromolecules e.g. antibodies, siRNAs, in liposomes or in nanoparticles (Fukuta et al. (2020) J Control Release 10: 323-332.)

In some embodiments, the compound is applied to the eyelid skin using a device. In some embodiments, the device is a dropper, a pump, a spray, a click pen or a tube reservoir device. In some embodiments, the compound is administered topically by brush, Q-tip, or spatula.

In some embodiments, the device releases a preselected dosage in a uniform manner onto the eyelid skin of the patient. In some embodiments, the compound is applied by a roller device to the eyelid skin. In some embodiments, the compound is applied by a Q-tip to the eyelid skin. In some embodiments, the compound is applied by a spatula to the eyelid skin. In some embodiments, the application process may be preceded by using a graduated dropper, syringe, click pen or pipette.

In some embodiments, penetration through the eyelid skin is increased by a penetration enhancer, tape-stripping, microdermabrasion, solvent, pulsed laser, and iontophoresis, which has been found useful for delivering macromolecules e.g. antibodies, siRNAs, in liposomes or in nanoparticles (Fukuta et al. (2020) J Control Release 10: 323-332).

In some embodiments, the compound is administered prophylactically, as an emergency intervention, or as required to achieve the desired remedial effects.

### Certain Definitions

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range may vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, may “consist of” or “consist essentially of” the described features.

“Treating” or “treatment” as used herein includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, delay or slowing of disease progression, amelioration, diminishment of the reoccurrence of disease. Treatment may prevent the disease from occurring; relieve the disease's symptoms, fully or partially remove the disease's underlying cause, shorten a disease's duration, or do a combination of the above.

“Treating” and “treatment” as used herein may also include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for duration sufficient to treat the patient.

The terms “effective amount,” “therapeutically effective amount” or “pharmaceutically effective amount” refer to an amount of an active agent effective to retinal diseases or other ophthalmic diseases, including a range of effects, from a detectable amount of improvement to substantial relief/improvement of symptoms or a cure of the disease or condition. The result can be a reduction and/or alleviation of the signs, symptoms, or causes of a disease, or any other desired alteration of a biological system. For example, an “effective amount” for therapeutic uses is the amount of the composition comprising an agent as set forth herein required to provide a clinically significant decrease in an ophthalmic disease. For example, for the given aspect (e.g., length of incidence), a therapeutically effective amount will show an increase or decrease of at least 5%, 10%, 15%, 20%, 25%, 40%, 50%, 60%, 75%, 80%, 90%, or 100%. Therapeutic efficacy can also be expressed as “-fold” increase or decrease. For example, a therapeutically effective amount can have at least a 1.2-fold, 1.5-fold, 2-fold, 5-fold, or more effect over a control. An appropriate “effective” amount in any individual case may b e determined using techniques, such as a dose escalation study.

The term “periorbital” refers to the area surrounding the socket of the eye.

The term “preorbital” refers to the area in front of the orbit or eye socket.

The term “eyelid” refers to movable folds of the skin over the eye.

“OD” refers to the right eye.

“OS” refers to the left eye.

“OU” refers to both eyes.

“Periorbital administration” involves administration to the periorbital skin and specifically excludes administration to the upper eyelid, lower eyelid, and eyelid margins.

The term “lotion” describes an emulsion liquid dosage form. This dosage form is generally for external application to the skin (US FDA Drug Nomenclature Monograph, number C-DRG-00201).

The term “cream” describes an emulsion semisolid dosage form, usually containing >20% water and volatiles and/or <50% hydrocarbons, waxes or polyols as the vehicle. A cream is more viscous than a lotion. This dosage form is generally for external application to the skin (US FDA Drug Nomenclature Monograph, number C-DRG-00201).

The term “ointment” describes a semisolid dosage form, usually containing <20% water and volatiles and/or >50% hydrocarbons, waxes or polyols as the vehicle. This dosage form is generally for external application to the skin or mucous membranes (US FDA Drug Nomenclature Monograph, number C-DRG-00201).

The term “solution” describes a clear, homogeneous liquid dosage form that contains one or more chemical substances dissolved in a solvent or mixture of mutually miscible solvents (US FDA Drug Nomenclature Monograph, number C-DRG-00201).

The term “suspension” refers to a heterogeneous mixture containing solid particles that are sufficiently large for sedimentation.

“Emulsion” means, but is not limited to, an oil-in-water emulsion, a water-in-oil emulsion, a micro emulsion referring to particle sizes of 10-9.

“Formulation” and “composition,” are intended to be equivalent and refer to a composition of matter suitable for pharmaceutical use (i.e., producing a therapeutic effect as well as possessing acceptable pharmacokinetic and toxicological properties).

“Emollient” is an agent that softens and soothes the skin.

Humectant” is a hygroscopic agent that moistens the skin.

“Penetration enhancer” is an agent that improves transdermal drug delivery.

“Thickening agent” increases the viscosity of a formulation to achieve optimal application characteristics.

“Ocular Surface” is the cornea and sclera and its associated bulbar conjunctiva

“Ophthalmic acceptable composition” is a composition that can be administered to the eye.

“Pharmaceutically acceptable” is used as equivalent to physiologically acceptable. In certain embodiments, a pharmaceutically acceptable composition or preparation will include agents for buffering and preservation in storage, and can include buffers and carriers for appropriate delivery, depending on the route of administration.

The terms “subject,” “patient,” “individual,” are not intended to be limiting and can be generally interchanged. That is, an individual described as a “patient” does not necessarily have a given disease, but may be merely seeking medical advice. The term “subject” as used herein includes all members of the animal kingdom prone to suffering from the indicated disorder. In some aspects, the subject is a mammal, and in some aspects, the subject is a human.

As used herein, “topical”, “topical application,” “topical administration,” and “topically administering” are used interchangeably herein and include the administration to the surface of the eye or to the periorbital skin of a subject, unless otherwise specified. Topical application or administering may result in the delivery of an active agent directly into the eye.

The term combination refers to separate entities used together to achieve improved or optimal therapeutic benefit and safety. In the simplest case, the combination may be a combination of two therapeutic agents at fixed doses administered concomitantly. In this case, the ingredients may be separately formulated or mixed together in a single formulation. However, to achieve satisfactory disease control, the doses of the therapeutic agents and the relative timing of their administration may require a degree of flexibility. For example, in a combination of two therapies, one therapy of the two may be administered first to establish its baseline level of remediation before the other (second) drug is added. A combination of drugs may involve administration of drugs by different formulations, dosing methods, and different routes of administration. As an illustrative examples, 4,5-dihydro-N-[4-[[4-(1-methylethoxy) phenyl] methyl] phenyl]-1H-imadazol-2-amine may be topically applied to the periorbital skin together with an intravitreal injection of a VEGF antibody, an intravenously administered VEGF antibody, or a VEGF antibody administered topically to the periorbital skin with or without 4,5-dihydro-N-[4-[[4-(1-methylethoxy) phenyl] methyl] phenyl]-1H-imadazol-2-amine present in the same formulation. Further permutations of delivery route and methods are contemplated.

“Topical formulation” and “topical pharmaceutical composition” are used interchangeably herein and include a formulation that is suitable for topical application to the eye. When specified, a topical formulation may be suitable for either topical application to the surface of the eye, to the periorbital skin of the eye, or both. A topical formulation may, for example, be used to confer a therapeutic benefit to its user.

The IP receptor is a cell surface protein that belongs to the G protein coupled receptor superfamily. The primary endogenous ligands for the IP receptor are prostacyclin (PGI2), prostaglandin E₁(PGE₁), and 19(S)-HETE (Woodward D, et al. (2011) Pharmacol Rev 63:471-538; Tunara S et al. (2016) PLOS one 11:0163633).

The platelet activating factor (PAF) receptor is also is a cell surface protein that belongs to the G protein coupled receptor superfamily (Ishii S et al. (2002) PGs & Other Lipid Med 68-69: 599-609).

SiRNA represents the same entity variously described as small interfering RNA, small inhibitory RNA, and short interfering RNA.

### EMBODIMENTS

Embodiment 1. A method of treating a disease or disorder of the posterior of the eye in a patient suffering from the disease or disorder comprising administering a therapeutically effective amount of an omega-3 fatty acid or a pharmaceutically acceptable ester or salt thereof to the exterior skin of the eyelid of an eye of the patient, wherein the omega-3 fatty acid is formulated for delivery to the posterior of the eye.

Embodiment 2. The method of Embodiment 1, wherein the disease or disorder of the posterior of the eye comprises a retinal disease.

Embodiment 3. The method of Embodiment 2, wherein the retinal disease comprises hemorrhage from the retinal or choroidal vasculature.

Embodiment 4. The method of Embodiment 3, wherein the hemorrhage is caused by systemic hypertension, diabetes, fatty liver disease, obesity, shaken baby syndrome, head trauma, anemia, or leukemia.

Embodiment 5. The method of Embodiment 2, wherein the retinal disease or disorder comprises plasma leakage from the retinal or choroidal vasculature.

Embodiment 6. The method of Embodiment 5, wherein the plasma leakage is caused by systemic hypertension, diabetes, fatty liver disease, obesity, shaken baby syndrome, head trauma, anemia, or leukemia.

Embodiment 7. The method of Embodiment 2, wherein the retinal disease or disorder comprises macular edema formation involving the retinal or choroidal vasculature.

Embodiment 8. The method of Embodiment 1, wherein the disease or disorder of the posterior of the eye is age-related macular degeneration (wet and dry forms), dry and wet macular degeneration, lattice Degeneration, macular hole, macular pucker, lattice degeneration, retinal tear, retinal detachment, retinal artery occlusion, retinal vein occlusion, central retinal vein occlusion, intraocular tumors, pediatric, neonatal or Inherited retinal disorders, hereditary retinal dystrophies, geographic atrophy, retinitis pigmentosa (including Leber congenital amaurosis), cytomegalovirus (cmv) retinal infection, infectious retinitis, retinoblastoma, endophthalmitis, chorioretinitis, myopic macular degeneration, and normal-tension glaucoma, retinal degeneration in glaucoma; various retinopathies, including but not limited to diabetic retinopathy, retinopathy of prematurity, Sickel cell retinopathy, radiation/solar retinopathy, central serous retinopathy, hypertensive retinopathy, peripheral retinopathy and neuropathy; macular edema, retinal hemorrhage, diabetic macular edema, diabetic macularischemia, geographic atrophy, Stargardt disease, uveitis (including intermediate uveitis, posterioruveitis, and panuveitis), or refractive errors (myopia, hyperopia, and astigmatism).

Embodiment 9. The method of any one of Embodiments 205-211, wherein the retinal disease or disorder is age-related macular degeneration.

Embodiment 10. The method of Embodiment 1, wherein the disease or disorder of the posterior of the eye is posterior uveitis.

Embodiment 11. The method of any one of Embodiments 1-10, further comprising administering to the patient an additional therapeutic agent.

Embodiment 12. The method of Embodiment 11, wherein the additional therapeutic agent is a VEGF antibody, a small molecule VEGF antagonist, a siRNA targeting a VEGF receptor, a TNFα antibody, a small molecule TNFα receptor antagonist, a siRNA targeting the TNFα receptor, an inflammatory cytokine receptor antagonist, an antibody against an inflammatory cytokine, a tyrosine kinase inhibitor, a serine/threonine-protein kinase inhibitors, a kinase inhibitor, a steroidal anti-inflammatory agent, a non-steroidal anti-inflammatory agent, an immunosuppressant, an anti-cholinergic agent, thalidomide, a prostaglandin receptor antagonist, a neuroprotective agent, a neurotrophic agent, a neuro-regenerative agent, an ocular hypotensive agent, an antibiotics, an antiviral agent, a complement inhibitor, an interleukin receptor inhibitor, a leukotriene receptor inhibitor, an inhibitor of tumorigenesis and development, an angiogenesis inhibitor, or agents with anti-oxidation or anti-microvascular leakage properties.

Embodiment 13. The method of any one of Embodiments 1-12, wherein the omega-3 fatty acid or a pharmaceutically acceptable ester or salt thereof is administered prophylactically, as an emergency intervention, or as required to achieve the desired remedial effects.

Embodiment 14. The method of any one of Embodiments 1-13, wherein the omega-3 fatty acid or a pharmaceutically acceptable ester or salt thereof is administered as a composition.

Embodiment 15. The method of Embodiment 1, wherein the omega-3 fatty acid or a pharmaceutically acceptable ester or salt thereof is present in an amount of about 0.0001% to about 10% (w/w) of the composition.

Embodiment 16. The method of Embodiment 14 or 15, wherein the composition is an aqueous solution, a non-aqueous solution, an oil solution, a gel, a suspension, an emulsion, a lotion, a cream, or an ointment.

Embodiment 17. The method of any one of Embodiments 14-16, wherein the composition is an ointment.

Embodiment 18. The method of Embodiment 17, wherein the ointment comprises petrolatum, beeswax, or cocoa butter.

Embodiment 19. The method of Embodiment 17 or 18, wherein the ointment comprises petrolatum and medium-chain triglycerides.

Embodiment 20. The method of Embodiment 19, wherein the medium-chain triglycerides comprise a mixture of C6, C8, C10 and C12 fatty acids.

Embodiment 21. The method of Embodiment 19 or 20, wherein the medium-chain triglycerides comprise a mixture of caprylic acid and capric acid.

Embodiment 22. The method of any one of Embodiments 17-21, wherein the ointment comprises petrolatum and medium-chain triglyceride in the ratio of about 1:1 (v/v), about 2:1 (v/v), about 3:1 (v/v), about 4:1 (v/v), about 5:1 (v/v), or about 6:1 (v/v).

Embodiment 23. The method of Embodiment 22, wherein the ointment comprises petrolatum and medium-chain triglyceride in the ratio of about 4:1 (v/v).

Embodiment 24. The method of any one of Embodiments 14-16, wherein the composition is an aqueous solution.

Embodiment 25. The method of Embodiment 24, wherein the aqueous solution comprises a polyoxyl castor oil.

Embodiment 26. The method of Embodiment 25, wherein the polyoxyl castor oil is a polyethylene glycol (PEG)-ylated castor oil.

Embodiment 27. The method of Embodiment 25 or 26, wherein the polyoxyl castor oil is polyoxyl 35 castor oil.

Embodiment 28. The method of Embodiment 27, wherein the polyoxyl 35 castor oil is present in an amount of about 0.1% to about 5%, about 0.1% to about 10%, about 0.1% to about 15%, or about 0.10% to about 20% (w/w) of the composition.

Embodiment 29. The method of any one of Embodiments 24-28, wherein the composition comprises an ocular surface lubricating agent.

Embodiment 30. The method of any one of Embodiments 1-29, wherein the omega-3 fatty acid or a pharmaceutically acceptable ester or salt thereof is applied to the exterior skin of the eyelid of an eye of the patient by dropper, pump, spray, click pen or roller/reservoir device.

Embodiment 31. The method of any one of Embodiments 1-29, wherein the omega-3 fatty acid or a pharmaceutically acceptable ester or salt thereof is applied to the exterior skin of the eyelid of an eye of the patient by brush, Q-tip, or spatula and where the application process is optionally preceded by using a graduated dropper, syringe, click pen or pipette.

Embodiment 32. The method any one of Embodiments 1-31 wherein eyelid skin penetration is assisted by tape-stripping, microdermabrasion, solvent, pulsed laser, iontophoresis, or combinations thereof.

Embodiment 33. The method of any one of Embodiments 1-32, wherein the omega-3 fatty acid or a pharmaceutically acceptable ester or salt thereof is administered to the eyelid skin of each eye of the patient four times per day, three times per day, twice per day, once per day, once every other day, once every three days, once every four days, or once every seven days.

Embodiment 34. The method of Embodiment 33, wherein the omega-3 fatty acid or a pharmaceutically acceptable ester or salt thereof is administered once per day.

Embodiment 35. The method of Embodiment 11, wherein the additional therapeutic agent is 4,5-dihydro-N-[4-[[4-(1-methylethoxy) phenyl] methyl] phenyl]-1H-imadazol-2-amine or a pharmaceutically acceptable ester or salt thereof.

Embodiment 36. The method of Embodiment 35, wherein the omega-3 fatty acid or a pharmaceutically acceptable ester or salt thereof and the 4,5-dihydro-N-[4-[[4-(1-methylethoxy) phenyl] methyl] phenyl]-1H-imadazol-2-amine or a pharmaceutically acceptable ester or salt thereof are formulated and administered as a single composition.

### EXAMPLES

### Example 1: Rabbit Study of JV-DE1 Topical Ophthalmic Administration to the Surface of Both Eyes

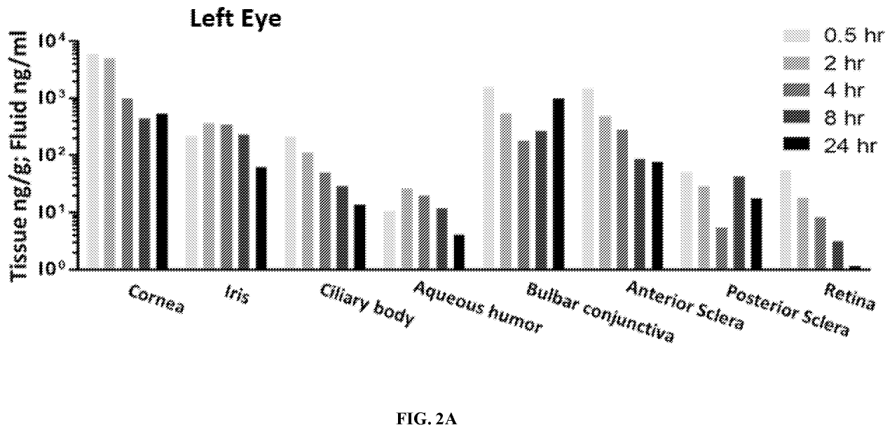

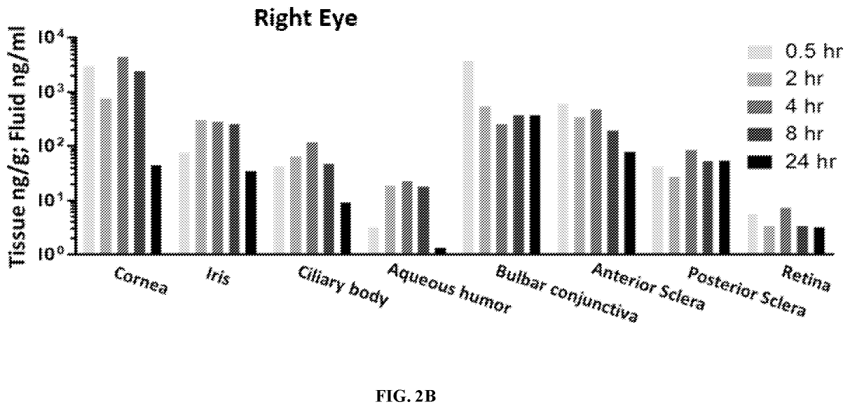

A retinal bioavailability study was performed on 4,5-dihydro-N-[4-[[4-(1-methylethoxy) phenyl] methyl] phenyl]-1H-imadazol-2-amine (assigned the coded notation JV-DE1). This study provided an indication that the compound formulated in an eye-drop and administered bilaterally to the anterior ocular surface was bioavailable in the retina. This pharmacokinetics and ocular tissue distribution study of JV-DE1 was performed in New Zealand rabbits. Briefly, 5 rabbits (male) received JV-DE1 via ocular instillation to both eyes at a dose of 0.12 mg/eye. Blood samples (1 mL) were collected at 0, 0.5, 2, 4, 8, and 24 h after single ocular administration to both eyes. At each time point, one animal was euthanized after blood collection and then eye tissue samples were collected from both eyes. Aqueous humor, bulbar conjunctiva, anterior sclera, posterior sclera (in the optic nerve exit region), retina, cornea, ciliary body, and iris were collected and weighed. The samples were then stored in an ultra-low temperature freezer. Drug concentrations in the samples were subsequently determined by the LC-MS/MS method. Results are shown in FIG. 2A (right eye) and FIG. 2B (left eye).

The concentration of JV-DE1 in plasma was low in New Zealand rabbits after ocular administration: the Cₘₐₓ was approximately 2 ng/mL at 0.5 hours post-dose, with good clearance at 24 hours post dose. JV-DE1 was widely distributed in the ocular tissues, and with no meaningful difference between the left and right eye, since drug administration was bilateral. JV-DE1 ocular tissue levels were well-maintained over an 8 hour period post-dosing but had declined by 24 hours. The cornea, anterior sclera, and bulbar conjunctiva had the highest levels of JV-DE1 in both eyes, whereas JV-DE1 levels were lower in those structures located within the globe. The most significant finding was the unexpectedly high levels of JV-DE1 in the retina and the posterior sclera, which underlies and supports the retina. These data, together the low plasma levels, provided a clear indication that JV-DE1 in the formulation, containing Polyoxyl 35 Castor Oil as an excipient, was capable of delivering therapeutically effective drug concentrations to the retina and posterior pole of the eye.

### Example 2: Rabbit Study of JV-DE1 Topical Ophthalmic Administration to the Surface of a Single Eye

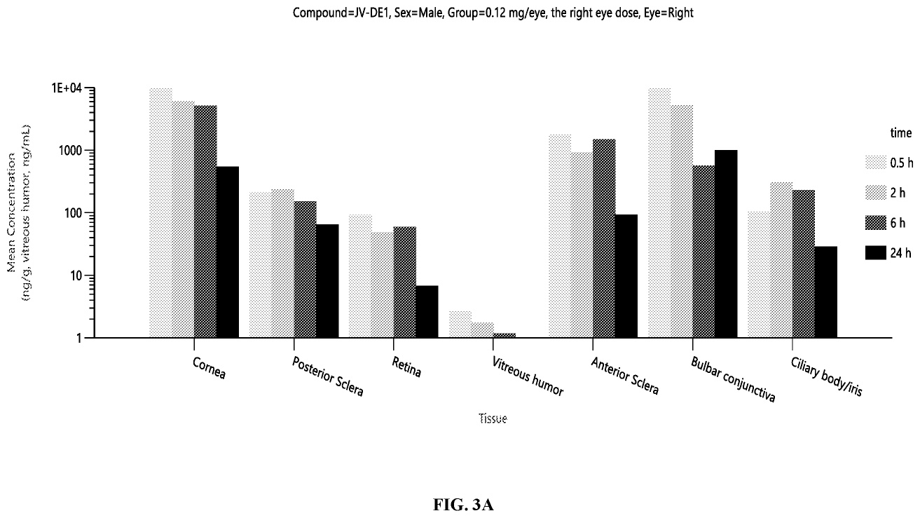

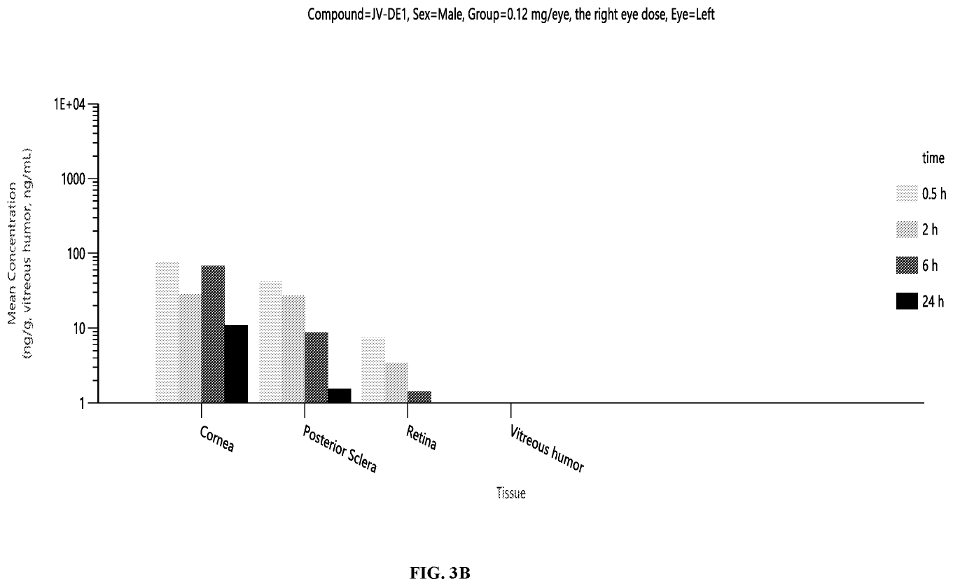

In order to verify that drug delivery of JV-DE1 was essentially confined to ocular redistribution other than via the blood circulation following contact of the eye-drop with the anterior segment ocular surface tissues, a monocular drug application study was performed. JV-DE1 was formulated in 2.4% CrEL and 0.2% glycerol in pH 7.6 TBS. CrEL is polyoxyl 35 castor oil. Other names for polyoxyl 35 castor oil include macrogolglycerol ricinoleate, PEG-35 castor oil, and polyoxyl 35 hydrogenated castor oil. Briefly, for the bio-disposition experiment, JV-DE1 was administered to one eye (OD) via ocular instillation to said eye at a dose of 0.12 mg/eye. Blood samples (1 mL) and were collected at 0, 0.5, 2, 6, and 24 h after a single eye-drop ocular administration. At each of the above time points, one animal was euthanized after blood collection and selected eye tissue samples were collected from both eyes. Aqueous humor, bulbar conjunctiva, anterior sclera, posterior sclera (in the optic nerve exit region), retina, cornea, ciliary body, and iris were collected and weighed. The samples were then stored in an ultra-low temperature freezer. JV-DE1 concentrations in the samples were subsequently determined by the LC-MS/MS method. FIG. 3A shows the concentration of JV-DE1 in various portions of the eye to which the JV-DE1 was administered at various time points. FIG. 3B shows the concentration of JV-DE1 in various portions of the eye which was not administered JV-DE1 at various time points after administration to the opposite eye.

The concentration of JV-DE1 in plasma was low in New Zealand rabbits after monocular administration: the Cₘₐₓ was 0.95 ng/mL at 0.5 hours post-dose with complete clearance by 24 hours post dose. JV-DE1 was widely distributed in the ocular tissues of the eye (OD) that received the compound. The concentration of JV-DE1 in ocular tissue levels were well-maintained over an 8 hour period post-dosing but had declined by 24 hours. The cornea, anterior sclera, and bulbar conjunctiva had the highest levels of JV-DE1 in the treated eye, which was about 100-fold greater than the JV-DE1 levels in the ciliary body/iris, posterior sclera, and retina. The most significant finding was the unexpected level of JV-DE1 in the vitreous humor. In the contralateral, untreated eye (OS), the compound was detected only in the cornea, posterior sclera, and retina. JV-DE1 levels in the retina of the contralateral, untreated eye were, however, one-tenth of those in the treated eye. These data, together the low plasma levels, indicated that 90% of the drug achieved the retina at pharmaceutical levels by intra-global redistribution of the eye-drop administered topically to the ocular surface. Only about 10% achieved the retina by delivery from the bloodstream.

### Example 3: Cynomolgus Monkey Study of JV-DE1 Administered to the Periorbital Skin of One Eye

An additional experiment on JV-DE1 involving determining its bio-disposition following topical application to the periorbital skin that surrounds the anterior portion of the globe was performed.

JV-DE1 was formulated in medium chain triglyceride (MCT) oil. An amount of about 0.16 mg of JV-DE1 formulation was administered to one eye (OD) by circumferential topical application to the periorbital skin that surrounds the anterior portion of the globe. Blood samples (1 mL) were collected at 0, 0.5, 2, 6, and 24 hours after a single administration to the periorbital skin of the right eye (OD) At each of the above pre-designated time points, one animal was euthanized after blood collection and selected eye tissue samples were collected from both eyes. The following representative tissues were collected; upper eyelid, cornea, retina, and vitreous humor. JV-DE1 concentrations in the samples were subsequently determined by the LC-MS/MS method. The results are summarized below in Table 1

TABLE 1
Biodistribution of JV-DE1 following a single dose right periorbital skin administration

| Test Article | | JV-DE1 | | | | |
|---|---|---|---|---|---|---|
| Dosing site | | Periorbital skin | | | | |
| Animal No. | | 101* | 101 | 102 | 103 | 104 |
| Time (h) | | 0 | 0.5 | 3 | 6 | 24 |
| Dose Level (μg/left eye) | | 0 | 0 | 0 | 0 | 0 |
| Dose Level (μg/right eye) | | 0 | 179.120 | 160.580 | 162.230 | 159.260 |
| Drug Concentration in Plasma (ng/mL) | | BLQ | 0.131 | 0.210 | 0.260 | BLQ |
| Drug Concentration in | Left upper eyelid | — | NA | NA | NA | NA |
| Tissue (ng/g) | Left cornea | — | NA | NA | NA | NA |
| | Left retina | — | NA | NA | NA | NA |
| | Left vitreous humor | — | NA | NA | NA | NA |
| | Right upper eyelid | — | 26535.400 | 32454.700 | 7945.600 | 11597.800 |
| | Right cornea | — | 39.600 | 27.900 | 45.500 | 5.500 |
| | Right retina | — | 9299.300 | 16688.700 | 23929.500 | 1621.300 |
| | Right vitreous humor | — | 15.200 | 7.790 | 3.900 | 18.970 |
| % of administered dose | Left upper eyelid | — | NA | NA | NA | NA |
| | Left cornea | — | NA | NA | NA | NA |
| | Left retina | — | NA | NA | NA | NA |
| | Left vitreous humor | — | NA | NA | NA | NA |
| | Right upper eyelid | — | 0.64 | 1.50 | 0.48 | 0.60 |
| | Right cornea | — | 0.00 | 0.00 | 0.00 | 0.00 |
| | Right retina | — | 0.09 | 0.26 | 0.25 | 0.05 |
| | Right vitreous humor | — | 4.67 | 2.91 | 3.13 | 1.79 |

The concentration of JV-DE1 in plasma was low in cynomolgus monkeys after a single dose monocular periorbital administration: the Cₘₐₓ was 0.13-0.26 ng/mL over the 6 hour post-dose period and with complete clearance by 24 hours post dose. The concentrations of JV-DE1 were highest in the upper eyelid (the site of application). JV-DE1 concentrations in the retina greatly exceeded those in the cornea from >200 fold to >500 fold.

The ocular distribution of JV-DE1 following application to the periorbital skin was quite different from that associated with conventional eye-drop administration to the ocular surface. Compound delivery to the posterior segment following periorbital administration was substantially greater than that to the cornea. In contrast, eye-drop administration delivered much more JV-DE1 to the cornea and other anterior segment tissues (FIG. 2A, 2B, 3A) than periorbital delivery. Nevertheless, both routes of administration were able to deliver therapeutically meaningful concentrations of JV-DE1 to the retina and the cornea, according to JV-DE1 pKi values obtained on human IP receptors and PAF receptors (Bley et al., 2006). The surprising discovery is that periorbital or eye-drop administration delivered JV-DE1 into the retina greatly exceeded the pKi values at IP and PAF receptors, such that complete antagonism would be achieved over a 24 hour period. The high and consistent levels of JV-DE1 delivered to the retina and vitreous humor suggest that a substantial quantity of drug delivered to the eye stays in the eye. The rapid accumulation of JV-DE1 in the vitreous humor and retina suggests an almost “open pathway” to certain drug penetration under certain conditions.

Bio-distribution of compounds delivered from the periorbital skin layers to the eyelids has not previously been the subject of experimental inquiry. It is known from a vast repository of patient experience that bimatoprost applied to the upper eyelid margin does not achieve the periorbital skin, since no hyperpigmentation induced skin discoloration above the eyelid margin occurs. Following application to the periorbital skin, high levels of JV-DE1 were achieved in the upper eyelids (See Table 1). JV-DE1 was administered via NIODP at the dose of 165.30±4.65 μg/eye (mean±SEM), to the right eye (OD) of monkeys in this pharmacokinetics and biodistribution study. For convenience of sample processing, only upper eyelids were collected. A substantial quantity of drug remained in the upper eyelid/periorbital region after 24 hours. A similar amount of drug, greater than 1000 ng/g (i.e. >1 μg/g), was found in the retina, and remained at such high level at all tested time points post dosing. On the other hand, drug levels were the lowest in cornea and vitreous humor. Drug levels retained in the retina were over 200-fold higher than in the cornea at each tested time point of 0.5, 3, 6 and 24-hour post dosing. JV-DE1 was at its maximal concentration of 23929.5 ng/g (23.9 μg/g) in retina at 6-hour post dosing, while the plasma concentrations remained less than 0.3 ng/ml, near the lower limit of quantitation (LLOQ, 0.1 ng/ml).

### Example 4: Cynomolgus Monkey Study of Docosahexaenoic Acid Administered Periorbitally

Delivery of compound to the posterior tissues of the eye, notably the retina, by application to the periorbital skin is not restricted or limited to JV-DE1. This is supported by results obtained with a markedly different compound docosahexaenoic acid (DHA), which are shown in Table 2. An oil solution comprising 14.85 mg/mL β-Carotene+198.02 mg/ml DHA in linoleic acid was administered to one eye (OD) by circumferential topical application to the periorbital skin that surrounds the anterior portion of the globe. Blood samples (1 mL) and were collected at 0, 0.5, 2, 6, and 24 hours after a single administration to the periorbital skin of the right eye (OD) At each of the above pre-designated time points, one animal was euthanized after blood collection and selected eye tissue samples were collected from both eyes. The following representative tissues were collected; upper eyelid, cornea, retina, and vitreous humor. DHA concentrations in the samples were determined by the LC-MS/MS method. The results are summarized below in Table 2. The DHA was administered via NIODP at the dose of 6775.1±92.5 μg per eye (mean±SEM), to the left eye of monkeys in the pharmacokinetics and biodistribution study. Again, for convenience of sample processing, only upper eyelids were collected. Substantial quantities of DHA (>10 μg/g) remained in the upper eyelid/periorbital region, retina, and cornea, at all tested time points of 0.5, 3, 6 and 24-hour post dosing. DHA rapidly reached its maximum of 110.9 g/gin retina at 0.5-hour post dosing. In vitreous humor, DHA remained below the limit of quantification (BLQ) with lower limit of quantitation (LLOQ) of 0.5 μg/ml (FIG. 3). Compared to the BLQ baseline, plasma concentrations of DHA were between 1.2 and 3.1 μg/ml, equivalent to 3.65 and 9.4 mM (MW 328.5) via NIODP. The plasma Cₘₐₓ was at 0.5 hours post-dose.

TABLE 2
Biodistribution of DHA following periorbital skin administration

| Test Article | | Docosahexaenoic acid (DHA) | | | | |
|---|---|---|---|---|---|---|
| Dosing site | | Periorbital skin | | | | |
| Animal No. | | 101* | 101 | 102 | 103 | 104 |
| Time (h) | | 0 | 0.5 | 3 | 6 | 24 |
| Dose Level (μg/left eye) | | 0 | 6567.89 | 6768.26 | 7017.66 | 6746.51 |
| Dose Level (μg/right eye) | | NA | NA | NA | NA | NA |
| Drug Concentration in Plasma (μg/mL) | | BLQ | 2.160 | 3.13 | 2.11 | 1.15 |
| Drug Concentration in | Left upper eyelid | — | 380.600 | 419.600 | 10.700 | 16.200 |
| Tissue (μg/g) | Left cornea | — | 66.300 | 21.600 | 22.300 | 25.900 |
| | Left retina | — | 110.900 | 66.700 | 78.600 | 47.300 |
| | Left vitreous humor | — | 0.000 | 0.000 | 0.000 | 0.000 |
| | Right upper eyelid | — | NA | NA | NA | NA |
| | Right cornea | — | NA | NA | NA | NA |
| | Right retina | — | NA | NA | NA | NA |
| | Right vitreous humor | — | NA | NA | NA | NA |
| % of administered dose | Left upper eyelid | — | 0.42 | 0.38 | 0.01 | 0.02 |
| | Left cornea | — | 0.03 | 0.01 | 0.01 | 0.02 |
| | Left retina | — | 0.04 | 0.04 | 0.01 | 0.03 |
| | Left vitreous humor | — | 0.00 | 0.00 | 0.00 | 0.00 |
| | Right upper eyelid | — | NA | NA | NA | NA |
| | Right cornea | — | NA | NA | NA | NA |
| | Right retina | — | NA | NA | NA | NA |
| | Right vitreous humor | — | NA | NA | NA | NA |

The plasma levels of docosahexaenoic acid (DHA) were essentially stable throughout the 24 hr experimental time course; 2.2, 3.1, 2.1, and 1.2 μg/mL at 0.5, 3, 6, and 24 hr post-dose, respectively. This plasma concentration of DHA was found significantly elevated to about 2 to 6-fold above its endogenous level (0.5 μg/ml) in monkeys. This means that NIODP delivery may also be beneficial to achieve higher systemic circulation of DHA for general health of human body. More DHA omega-3 clinical trials for AMD treatment, and other retinal diseases should be conducted using the NIODP route. NIODP has the potential to abolish or significantly reduce the need of injection to the eye, so that to disrupt the current method of choice for retinal drug delivery. In comparison with JV-DE1, DHA was rapidly penetrated into ocular tissues and was rapidly reduced in site of application in the upper eyelid.

Conversely, the beta-carotene component of the formulation was not as readily uptaken and biodistributed following administration, as shown in Table 3. Thus, not all compounds are readily uptaken and distributed to the posterior area of the eye.

TABLE 3
Biodistribution of Beta-Carotene following periorbital skin administration

| Test Article | | Beta(β)-Carotene | | | | |
|---|---|---|---|---|---|---|
| Dosing site | | Periorbital skin | | | | |
| Animal No. | | 101* | 101 | 102 | 103 | 104 |
| Time (h) | | 0 | 0.5 | 3 | 6 | 24 |
| Dose Level (μg/left eye) | | 0 | 492.59 | 507.60 | 526.32 | 505.99 |
| Dose Level (μg/right eye) | | 0 | 0 | 0 | 0 | 0 |
| Drug Concentration in Plasma (μg/mL) | | BLQ | BLQ | BLQ | BLQ | BLQ |
| Drug Concentration in | Upper eyelid | — | 12.600 | 36.000 | 0.000 | 0.000 |
| Tissue (μg/g) | Cornea | — | 0.000 | 0.000 | 0.000 | 0.000 |
| | Retina | — | 0.000 | 0.000 | 0.000 | 0.000 |
| | Vitreous humor | — | 0.000 | 0.000 | 0.000 | 0.000 |
| | Right upper eyelid | — | NA | NA | NA | NA |
| | Right cornea | — | NA | NA | NA | NA |
| | Right retina | — | NA | NA | NA | NA |
| | Right vitreous humor | — | NA | NA | NA | NA |
| % of administered dose | Left Upper eyelid | — | 0.19 | 0.44 | 0.000 | 0.000 |
| | Left Cornea | — | 0.000 | 0.000 | 0.000 | 0.000 |
| | Left Retina | — | 0.000 | 0.000 | 0.000 | 0.000 |
| | Left Vitreous humor | — | 0.000 | 0.000 | 0.000 | 0.000 |
| | Right upper eyelid | — | NA | NA | NA | NA |
| | Right cornea | — | NA | NA | NA | NA |
| | Right retina | — | NA | NA | NA | NA |
| | Right vitreous humor | — | NA | NA | NA | NA |

The administration of compounds applied to the periorbital skin surprisingly provides therapeutically and beneficially effective amounts to the ocular posterior segment, as well as to the ocular surface and the eyelids. Periorbital application was particularly advantageous in supplying compounds to the retina. The routes of administration depicted, albeit not to be limited by theory, suggest that favored penetration into the vitreous humor and retina following periorbital administration involves by passing the lens, which for eye-drops, provides a major obstruction to the passage of drugs from the anterior ocular segment to the ocular posterior segment that includes the retina.

### Example 5: Non-Invasive Periorbital Ocular Drug Delivery

Drugs are dissolved and delivered via a non-invasive and non-irritating formulation to both the anterior segment of the eye and the posterior segment of the eye by administration to the periorbital skin of an eye. Drugs that are lipophilic are preferably transported to these segments of the eye; once the drugs pass the stratum corneum of the periorbital skin, the drugs may undergo passive diffusion via the conjunctiva into the scleral water channel. Drugs that may be ionizable at physiological pH in the scleral water channel are preferably transported to the posterior segment of the eye, overcoming limitations posed by the protective anatomical and physiological barriers that limit access to the retina.

### Example 6: Endogenous DHA Levels in Ocular Tissues

The biodistribution of DHA in ocular tissues following periorbital skin administration, shown in Table 2, is not due to endogenous levels of DHA (DHA originating from within an organism and not attributable to any external factor). Endogenous levels of DHA within ocular tissues are negligible. Table 4 shows the endogenous levels of DHA present in ocular tissues, measured in μg/mL. Prior to the measurements in Table 4, patients were not dosed with any drug formulations.

TABLE 4
Results of DHA in Blank Matrix

| | | | Analyte | Analyte | IS Peak | Calculated | |
|---|---|---|---|---|---|---|---|
| | | Dilution | Concentration | Peak Area | Area | Concentration | Accuracy |
| Sample Name | Sample Type | Factor | (ng/mL) | (counts) | (counts) | (μg/mL) | (%) |
| Double blank-1- | Double Blank | 1 | 0 | 2.48E+04 | N/A | 1.63 | N/A |
| retina | | | | | | | |
| Double blank-2- | Double Blank | 1 | 0 | 2.65E+04 | N/A | 1.74 | N/A |
| retina | | | | | | | |
| Double blank-1- | Double Blank | 1 | 0 | 0.00E+00 | N/A | BLQ | N/A |
| upper eyelid | | | | | | | |
| Double blank-2- | Double Blank | 1 | 0 | 0.00E+00 | N/A | BLQ | N/A |
| upper eyelid | | | | | | | |
| Double blank-1- | Double Blank | 1 | 0 | 0.00E+00 | N/A | BLQ | N/A |
| cornea | | | | | | | |
| Double blank-2- | Double Blank | 1 | 0 | 0.00E+00 | N/A | BLQ | N/A |
| cornea | | | | | | | |
| Double blank-1- | Double Blank | 1 | 0 | 1.20E+04 | N/A | 0.53 | N/A |
| plasma | | | | | | | |
| Double blank-2- | Double Blank | 1 | 0 | 1.36E+04 | N/A | 0.61 | N/A |
| plasma | | | | | | | |
| Double blank-1- | Double Blank | 1 | 0 | 0.00E+00 | N/A | BLQ | N/A |
| vitreous humor | | | | | | | |
| Double blank-2- | Double Blank | 1 | 0 | 0.00E+00 | N/A | BLQ | N/A |
| vitreous humor | | | | | | | |

BLQ: Below the lower limit of quantification (LLOQ = 0.5 μg/mL) and given a value of 0 in relevant calculations.

### Example 7: Endogenous Beta-Carotene Levels in Ocular Tissues

The biodistribution of Beta-Carotene in ocular tissues following periorbital skin administration, shown in Table 3, is not due to endogenous levels of Beta-Carotene (Beta-Carotene) originating from within an organism and not attributable to any external factor). Endogenous levels of Beta-Carotene within ocular tissues is negligible. Table 5 shows the endogenous levels of Beta-Carotene present in ocular tissues, measured in gig/mL. Prior to the measurements in Table 5, patients were not dosed with any drug formulations.

TABLE 5
Results of Beta-Carotene in Blank Matrix

| | | | Analyte | Analyte | IS Peak | Calculated | |
|---|---|---|---|---|---|---|---|
| | | Dilution | Concentration | Peak Area | Area | Concentration | Accuracy |
| Sample Name | Sample Type | Factor | (ng/mL) | (counts) | (counts) | (μg/mL) | (%) |
| Double blank-1- | Double Blank | 1 | 0 | 1.44E+05 | N/A | 1.15 | N/A |
| retina | | | | | | | |
| Double blank-2- | Double Blank | 1 | 0 | 0.00E+00 | N/A | BLQ | N/A |
| retina | | | | | | | |
| Double blank-1- | Double Blank | 1 | 0 | 3.26E+04 | N/A | BLQ | N/A |
| upper eyelid | | | | | | | |
| Double blank-2- | Double Blank | 1 | 0 | 0.00E+00 | N/A | BLQ | N/A |
| upper eyelid | | | | | | | |
| Double blank-1- | Double Blank | 1 | 0 | 7.10E+04 | N/A | BLQ | N/A |
| cornea | | | | | | | |
| Double blank-2- | Double Blank | 1 | 0 | 2.37E+03 | N/A | BLQ | N/A |
| cornea | | | | | | | |
| Double blank-1- | Double Blank | 1 | 0 | 3.31E+03 | N/A | BLQ | N/A |
| plasma | | | | | | | |
| Double blank-2- | Double Blank | 1 | 0 | 3.81E+02 | N/A | BLQ | N/A |
| plasma | | | | | | | |
| Double blank- | Double Blank | 1 | 0 | 0.00E+00 | N/A | BLQ | N/A |
| vitreous humor | | | | | | | |
| Double blank- | Double Blank | 1 | 0 | 0.00E+00 | N/A | BLQ | N/A |
| vitreous humor | | | | | | | |

BLQ: Below the lower limit of quantification (LLOQ = 0.5 μg/mL) and given a value of 0 in relevant calculations.

### Example 8: Non-Invasive Eyelid Ocular Drug Delivery

Drugs are dissolved and delivered via a non-invasive and non-irritating formulation to both the anterior segment of the eye and the posterior segment of the eye by administration to the eyelid skin of an eye. Drugs that are lipophilic are preferably transported to these segments of the eye; once the drugs pass the stratum corneum of the eyelid skin, the drugs may undergo passive diffusion via the conjunctiva into the scleral water channel. For example, DHA and JV-DE1 are both lipophilic, aka both compounds have a Predicted Lipophilicity Indicator greater than or equal to one. The Predicted Lipophilicity Indicator (Log P value) for DHA is around 6.8. The Predicted Lipophilicity Indicator (Log P value) for JV-DE1 is around 3.36. Drugs that may be ionizable at physiological pH in the scleral water channel are preferably transported to the posterior segment of the eye, overcoming limitations posed by the protective anatomical and physiological barriers that limit access to the retina. For example, DHA has a physiological charge of −1. Similarly, JV-DE1 has a physiological charge of +1. Compounds with more than one ionizable center at physiological pH are more likely to reach the posterior segment of the eye.

### Example 9: Comparison of Non-Invasive Ocular Delivery Platform with Eye Drops for Drug Delivery of JV-DE1 and DHA

A non-invasive ocular delivery platform (NIODP) is a combination of periorbital skin transdermal administration with appropriate drug formulation to deliver ocular drugs, particularly retinal drugs, at above μg/g of ocular tissue. It was demonstrated that high doses (>1 μg/g) of JV-DE1 with ocular anti-inflammatory and anti-microvascular leakage properties, can be delivered to the anterior chamber of the eye as an eye drop, and via NIODP to the posterior chamber of the eye, especially retina. The biodistribution dose gradient of JV-DE1 administered as eye drops was, cornea≥conjunctiva≥anterior sclera≈iris≥ciliary body≥posterior sclera≥aqueous humor≥retina. The biodistribution via NIODP was, eyelid≥retina>>cornea≥vitreous humor. A docosahexaenoic acid (DHA) of the omega-3 antioxidants, was also administered via NIODP. For DHA, the dose gradient for administration via NIODP was, eyelid≈retina>cornea>>vitreous humor=0. Although JV-DE1 administered as an eye drop achieved high concentrations in the cornea, it became trapped at iris and ciliary body, and the concentration in the anterior aqueous humor remained very low (almost as low as in the retina), and appeared to have lost the momentum of further diffusion passing the vitreous humor to the retina. On the other hand, JV-DE1 remained at a good concentration gradient through the sclera pathway from the conjunctiva through posterior sclera to the retina. Therefore, the less than 60 ng/g retina distribution of JV-DE1 eye-drop is likely through the sclera pathway rather than via the cornea pathway.

Similarly, JV-DE1 and DHA also seem to reach the retina via the sclera pathway when delivered by NIODP, because drug in the vitreous humor was either quite low (JV-DE1) or BLQ (DHA), even when drug concentrations were high in the cornea (DHA). This observation concurs with prior reports that via conjunctival/scleral injection (underneath the periorbital skin), non-ionic nomicelles were more able to diffuse through the scleral water channels to reach the retina than by diffusing through the cornea pathway to the retina. The scleral water channels are a network of collagen fibers, proteoglycans, and glycoproteins in an aqueous medium. Depending on the drug physicochemical properties, such as molecular weight, radius, charge and lipophilicity, the sclera pathway provides an easier route for certain drugs to bypass the anterior segment barriers (the lens, iris, and ciliary body), and transport drugs to the back of the eye.

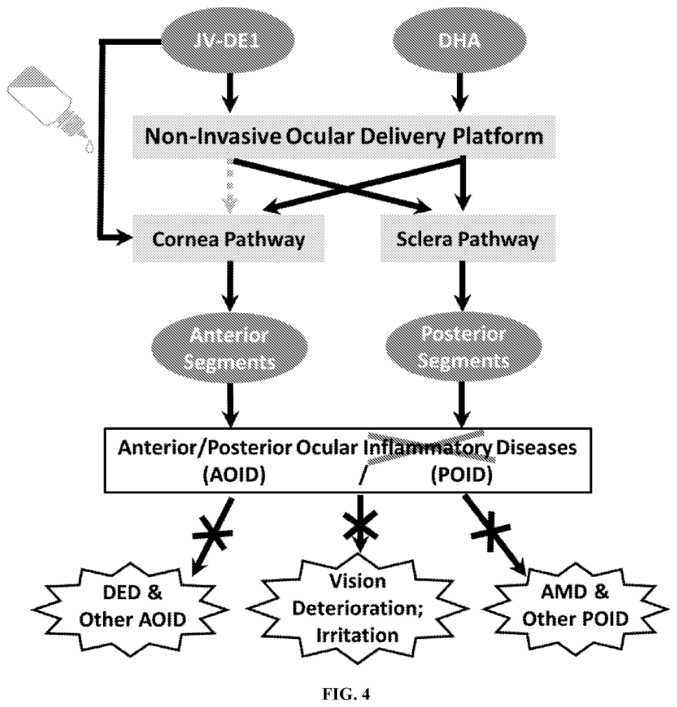

As summarized in FIG. 4, when administered via the eye-drop route, most drugs transport through the cornea pathway, and only sometimes reach the retina in negligible levels. When applied via the NIODP route, however, once penetrated through the stratum corneum of periorbital skin, drugs can either passively diffuse through the scleral water channels (the “sclera pathway”) to retina or take the cornea pathway to the anterior segments. The NIODP can deliver compounds at high doses via the cornea route to the anterior segments, and via the sclera pathway to the posterior segments, while eye drops deliver drug (JV-DE1) mostly via the cornea pathway to anterior segments of the eye. The effective drug concentration in the target tissue determines the success of treatment of the targeted diseases, such as dry eye disease (DED) and other anterior ocular inflammatory diseases (AOID), age-related macular de-generation (AMD), other posterior ocular inflammatory diseases (POID) and neurodegenerative ocular diseases, as well as prevention of progression of the resulting vison deterioration and irritation associated with these inflammatory and neurodegenerative diseases.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A method of treating a disease or disorder of the posterior of the eye in a patient suffering from the disease or disorder comprising administering a therapeutically effective amount of 4,5-dihydro-N-[4-[[4-(1-methylethoxy) phenyl] methyl] phenyl]-1H-imadazol-2-amine or a pharmaceutically acceptable ester or salt thereof to the periorbital skin of an eye of the patient.

2. The method of claim 1, wherein the disease or disorder of the posterior of the eye comprises a retinal disease.

3. The method of claim 2, wherein the retinal disease comprises hemorrhage from the retinal or choroidal vasculature.

4. The method of claim 3, wherein the hemorrhage is caused by systemic hypertension, diabetes, fatty liver disease, obesity, shaken baby syndrome, head trauma, anemia, or leukemia.

5. The method of claim 2, wherein the retinal disease or disorder comprises plasma leakage from the retinal or choroidal vasculature.

6. The method of claim 5, wherein the plasma leakage is caused by systemic hypertension, diabetes, fatty liver disease, obesity, shaken baby syndrome, head trauma, anemia, or leukemia.

7. The method of claim 2, wherein the retinal disease or disorder comprises macular edema formation involving the retinal or choroidal vasculature.

8. The method of claim 1, wherein the disease or disorder of the posterior of the eye is age-related macular degeneration (wet and dry forms), dry and wet macular degeneration, lattice Degeneration, macular hole, macular pucker, lattice degeneration, retinal tear, retinal detachment, retinal artery occlusion, retinal vein occlusion, central retinal vein occlusion, intraocular tumors, pediatric, neonatal or inherited retinal disorders, hereditary retinal dystrophies, geographic atrophy, retinitis pigmentosa (including Leber congenital amaurosis), cytomegalovirus (cmv) retinal infection, infectious retinitis, retinoblastoma, endophthalmitis, chorioretinitis, myopic macular degeneration, and normal-tension glaucoma, retinal degeneration in glaucoma, various retinopathies, including but not limited to diabetic retinopathy, retinopathy of prematurity, Sickel cell retinopathy, radiation/solar retinopathy, central serous retinopathy, hypertensive retinopathy, peripheral retinopathy and neuropathy, macular edema, retinal hemorrhage, diabetic macular edema, diabetic macular ischemia, geographic atrophy, Stargardt disease, uveitis (including intermediate uveitis, posterior uveitis, and panuveitis) or refractive errors (myopia, hyperopia, and astigmatism).

9. The method of any one of claims 2-8, wherein the retinal disease or disorder is age-related macular degeneration.

10. The method of claim 1, wherein the disease or disorder of the posterior of the eye is posterior uveitis.

11. The method of any one of claims 1-10, further comprising administering to the patient an additional therapeutic agent.

12. The method of claim 11, wherein the additional therapeutic agent is a VEGF antibody, a small molecule VEGF antagonist, a siRNA targeting a VEGF receptor, a TNFα antibody, a small molecule TNFα receptor antagonist, a siRNA targeting the TNFα receptor, an inflammatory cytokine receptor antagonist, an antibody against an inflammatory cytokine, a tyrosine kinase inhibitor, a serine/threonine-protein kinase inhibitors, a kinase inhibitor, a steroidal anti-inflammatory agent, a non-steroidal anti-inflammatory agent, an immunosuppressant, an anti-cholinergic agent, thalidomide, a prostaglandin receptor antagonist, a neuroprotective agent, a neurotrophic agent, a neuro-regenerative agent, an ocular hypotensive agent, an antibiotics, an antiviral agent, a complement inhibitor, an interleukin receptor inhibitor, a leukotriene receptor inhibitor, an inhibitor of tumorigenesis and development, an angiogenesis inhibitor, or agents with anti-oxidation or anti-microvascular leakage properties.

13. The method of any one of claims 1-12, wherein the 4,5-dihydro-N-[4-[[4-(1-methylethoxy) phenyl] methyl] phenyl]-1H-imadazol-2-amine is administered prophylactically, as an emergency intervention, or as required to achieve the desired remedial effects.

14. The method of any one of claims 1-13, wherein the 4,5-dihydro-N-[4-[[4-(1-methylethoxy) phenyl] methyl] phenyl]-1H-imadazol-2-amine is administered as a composition.

15. The method of claim 14, wherein the 4,5-dihydro-N-[4-[[4-(1-methylethoxy) phenyl]methyl] phenyl]-1H-imadazol-2-amine is present in an amount of about 0.00010% to about 10% (w/w) of the composition.

16. The method of claim 14 or 15, wherein the composition is an aqueous solution, a non-aqueous solution, an oil solution, a gel, a suspension, an emulsion, a lotion, a cream, or an ointment.

17. The method of any one of claims 14-16, wherein the composition is an ointment.

18. The method of claim 17, wherein the ointment comprises petrolatum, beeswax, or cocoa butter.

19. The method of claim 17 or 18, wherein the ointment comprises petrolatum and medium-chain triglycerides.

20. The method of claim 19, wherein the medium-chain triglycerides comprise a mixture of C6, C8, C10 and C12 fatty acids.

21. The method of claim 19 or 20, wherein the medium-chain triglycerides comprise a mixture of caprylic acid and capric acid.

22. The method of any one of claims 17-21, wherein the ointment comprises petrolatum and medium-chain triglyceride in the ratio of about 1:1 (v/v), about 2:1 (v/v), about 3:1 (v/v), about 4:1 (v/v), about 5:1 (v/v), or about 6:1 (v/v).

23. The method of claim 22, wherein the ointment comprises petrolatum and medium-chain triglyceride in the ratio of about 4:1 (v/v).

24. The method of any one of claims 14-16, wherein the composition is an aqueous solution.

25. The method of claim 24, wherein the aqueous solution comprises a polyoxyl castor oil.

26. The method of claim 25, wherein the polyoxyl castor oil is a polyethylene glycol (PEG)-ylated castor oil.

27. The method of claim 25 or 26, wherein the polyoxyl castor oil is polyoxyl 35 castor oil.

28. The method of claim 27, wherein the polyoxyl 35 castor oil is present in an amount of about 0.10% to about 5%, about 0.1% to about 10%, about 0.1% to about 15%, or about 0.1% to about 20% (w/w) of the composition.

29. The method of any one of claims 24-28, wherein the composition comprises an ocular surface lubricating agent.

30. The method of any one of claims 1-29, wherein the 4,5-dihydro-N-[4-[[4-(1-methylethoxy) phenyl] methyl] phenyl]-1H-imadazol-2-amine is applied to the periorbital skin of at least one eye of the patient by dropper, pump, spray, click pen or roller/reservoir device.

31. The method of any one of claims 1-29, wherein the 4,5-dihydro-N-[4-[[4-(1-methylethoxy) phenyl] methyl] phenyl]-1H-imadazol-2-amine is applied to the periorbital skin of at least one eye of the patient by brush, Q-tip, or spatula and where the application process is optionally preceded by using a graduated dropper, syringe, click pen or pipette.

32. The method any one of claims 1-31 wherein periorbital skin penetration is assisted by tape-stripping, microdermabrasion, solvent, pulsed laser, iontophoresis, or combinations thereof.

33. The method of any one of claims 1-32, wherein the 4,5-dihydro-N-[4-[[4-(1-methylethoxy) phenyl] methyl] phenyl]-1H-imadazol-2-amine is administered to the periorbital skin of each eye of the patient four times per day, three times per day, twice per day, once per day, once every other day, once every three days, once every four days, or once every seven days.

34. The method of claim 33, wherein the 4,5-dihydro-N-[4-[[4-(1-methylethoxy) phenyl]methyl] phenyl]-1H-imadazol-2-amine is administered once per day.

35. The method of any one of claims 1-34, wherein the method comprises administering the composition to the periorbital skin above the upper eyelid, below the lower eyelid, or both above the upper and below the lower eyelids.

36. The method of claim 11, wherein the additional therapeutic agent is an omega-3 fatty acid or a pharmaceutically acceptable ester or salt thereof.

37. The method of claim 36, wherein the 4,5-dihydro-N-[4-[[4-(1-methylethoxy) phenyl]methyl] phenyl]-1H-imadazol-2-amine and the omega-3 fatty acid or a pharmaceutically acceptable ester or salt thereof are formulated and administered as a single composition.

38. A method of treating a disease or disorder of the posterior of the eye in a patient suffering from the disease or disorder comprising administering a therapeutically effective amount of 4,5-dihydro-N-[4-[[4-(1-methylethoxy) phenyl] methyl] phenyl]-1H-imadazol-2-amine or a pharmaceutically acceptable ester or salt thereof to the ocular surface of an eye of the patient.

39. The method of claim 38, wherein the disease or disorder of the posterior of the eye comprises a retinal disease.

40. The method of claim 39, wherein the retinal disease comprises hemorrhage from the retinal or choroidal vasculature.

41. The method of claim 40, wherein the hemorrhage is caused by systemic hypertension, diabetes, fatty liver disease, obesity, shaken baby syndrome, head trauma, anemia, or leukemia.

42. The method of claim 38, wherein the retinal disease or disorder comprises plasma leakage from the retinal or choroidal vasculature.

43. The method of claim 42, wherein the plasma leakage is caused by systemic hypertension, diabetes, fatty liver disease, obesity, shaken baby syndrome, head trauma, anemia, or leukemia.

44. The method of claim 39, wherein the retinal disease or disorder comprises macular edema formation in the retinal or choroidal vasculature.

45. The method of claim 38, wherein the disease or disorder of the posterior of the eye is age-related macular degeneration (wet and dry forms), dry and wet macular degeneration, lattice Degeneration, macular hole, macular pucker, lattice degeneration, retinal tear, retinal detachment, retinal artery occlusion, retinal vein occlusion, central retinal vein occlusion, intraocular tumors, pediatric, neonatal or Inherited retinal disorders, hereditary retinal dystrophies, geographic atrophy, retinitis pigmentosa (including Leber congenital amaurosis), cytomegalovirus (cmv) retinal infection, infectious retinitis, retinoblastoma, endophthalmitis, chorioretinitis, myopic macular degeneration, and normal-tension glaucoma, retinal degeneration in glaucoma; various retinopathies, including but not limited to diabetic retinopathy, retinopathy of prematurity, Sickel cell retinopathy, radiation/solar retinopathy, central serous retinopathy, hypertensive retinopathy, peripheral retinopathy and neuropathy; macular edema, retinal hemorrhage, diabetic macular edema, diabetic macular ischemia, geographic atrophy, Stargardt disease, uveitis (including intermediate uveitis, posterior uveitis, and panuveitis), or refractive errors (myopia, hyperopia, and astigmatism).

46. The method of any one of claims 39-45, wherein the retinal disease or disorder is age-related macular degeneration.

47. The method of claim 38, wherein the disease or disorder of the posterior of the eye is posterior uveitis.

48. The method of any one of claims 38-47, further comprising administering to the patient an additional therapeutic agent.

49. The method of claim 48 wherein the additional therapeutic agent is a small molecule VEGF antagonist, a siRNA targeting A VEGF receptor, a small molecule TNFα receptor antagonist, a siRNA targeting the TNFα receptor, an inflammatory cytokine receptor antagonist, a tyrosine kinase inhibitor, a serine/threonine-protein kinase inhibitors, a kinase inhibitor, a steroidal anti-inflammatory agent, a non-steroidal anti-inflammatory agent, an immunosuppressant, an anti-cholinergic agent, thalidomide, a prostaglandin receptor antagonist, a neuroprotective agent, a neuro-regenerative agent, an ocular hypotensive agent, an antibiotics, an antiviral agent, a complement inhibitor, an interleukin receptor inhibitor, a leukotriene receptor inhibitor, an inhibitor of tumorigenesis and development, an angiogenesis inhibitor, or agents with anti-oxidation or anti-microvascular leakage properties.

50. The method of any one of claims 38-49, wherein the 4,5-dihydro-N-[4-[[4-(1-methylethoxy) phenyl] methyl] phenyl]-1H-imadazol-2-amine is administered as a composition, wherein the composition is an aqueous solution, a non-aqueous solution, an oil solution, a gel, a suspension, an emulsion, a cream, an ointment, in liposomes or in nanoparticles.

51. The method of claim 50, wherein the 4,5-dihydro-N-[4-[[4-(1-methylethoxy) phenyl]methyl] phenyl]-1H-imadazol-2-amine is present in an amount of about 0.0001% to about 10% (w/w) of the composition.

52. The method of claim 50 or 51, wherein the composition is an aqueous solution.

53. The method of claim 52, wherein the aqueous solution comprises a polyoxyl castor oil.

54. The method of claim 53, wherein the polyoxyl castor oil is a polyethylene glycol (PEG)-ylated castor oil.

55. The method of claim 53 or 54, wherein the polyoxyl castor oil is polyoxyl 35 castor oil.

56. The method of claim 55, wherein the polyoxyl 35 castor oil is present in an amount of about 0.1% to about 5%, about 0.1% to about 10%, about 0.1% to about 15%, or about 0.1% to about 20% (w/w) of the composition.

57. The method of any one of claims 53-55, wherein the composition comprises an ocular surface lubricating agent.

58. The method of any one of claims 38-57, wherein the 4,5-dihydro-N-[4-[[4-(1-methylethoxy) phenyl] methyl] phenyl]-1H-imadazol-2-amine is administered to the ocular surface of each eye of the patient four times per day, three times per day, twice per day, once per day, once every other day, once every three days, once every four days, or once every seven days.

59. The method of claim 58, wherein the 4,5-dihydro-N-[4-[[4-(1-methylethoxy) phenyl]methyl] phenyl]-1H-imadazol-2-amine is administered once per day.

60. The method of claim 48, wherein the additional therapeutic agent is an omega-3 fatty acid or a pharmaceutically acceptable ester or salt thereof.

61. The method of claim 60, wherein the omega-3 fatty acid or a pharmaceutically acceptable ester or salt thereof is administered periorbitally as a composition.

62. A method of treating uveitis in a patient suffering from uveitis comprising administering to the eye of the patient a therapeutically effective amount of 4,5-dihydro-N-[4-[[4-(1-methylethoxy) phenyl] methyl] phenyl]-1H-imadazol-2-amine or a pharmaceutically acceptable ester or salt thereof.

63. A method of treating pterygium in a patient suffering from pterygium comprising administering to the eye of the patient a therapeutically effective amount of 4,5-dihydro-N-[4-[[4-(1-methylethoxy) phenyl] methyl] phenyl]-1H-imadazol-2-amine or a pharmaceutically acceptable ester or salt thereof.

64. A method of treating an ocular disease or disorder in a patient suffering from the disease or disorder, comprising administering to the eye of the patient a therapeutically effective amount of 4,5-dihydro-N-[4-[[4-(1-methylethoxy) phenyl] methyl] phenyl]-1H-imadazol-2-amine or a pharmaceutically acceptable ester or salt thereof, wherein the ocular disease or disorder is anterior segment dysgenesis, cataract, iritis, pterygium, keratoconjunctivitis, keratitis, conjunctivitis, keratoconus, ectatic disorders (including keratoglobus, pellucid marginal degeneration), Pseudophakic and aphakic bullous keratopathy, episcleritis, corneal ulceration, corneal dysplasia, corneal ulceration, Fuchs' endothelial dystrophy and other corneal dystrophies (including lattice, granular, macular, and map-dot fingerprint), ocular cicatricial pemphigoid, Stevens Johnson syndrome, acute and chronic uveitis (anterior uveitis, intermediate uveitis), trauma to the cornea, conjunctiva and anterior segment including iris trauma, or penetrating ocular trauma.

65. A method of treating an ocular disease or disorder affecting the eyelid of a patient suffering from the disease or disorder, comprising administering to the eye of the patient a therapeutically effective amount of 4,5-dihydro-N-[4-[[4-(1-methylethoxy) phenyl] methyl]phenyl]-1H-imadazol-2-amine or a pharmaceutically acceptable ester or salt thereof, wherein the ocular disease or disorder affecting the eyelid is blepharitis, blepharospasm, chalazion, ptosis, coloboma, dermatochalasis, ectropion, entropion, trichiasis, stye, meibomianitis, Meibomian Gland Dysfunction, lacrimal gland obstruction, lacrimal gland obstruction, seborrheic keratitis, actinic keratitis, bacterial infection, or viral infection.

66. The method of any one of claims 62-65, wherein the 4,5-dihydro-N-[4-[[4-(1-methylethoxy) phenyl] methyl] phenyl]-1H-imadazol-2-amine is administered topically to the surface of the eye as a composition.

67. The method of any one of claims 62-66, wherein the 4,5-dihydro-N-[4-[[4-(1-methylethoxy) phenyl] methyl] phenyl]-1H-imadazol-2-amine is administered periorbitally as a composition.

68. The method of any one of claims 62-67, wherein the 4,5-dihydro-N-[4-[[4-(1-methylethoxy) phenyl] methyl] phenyl]-1H-imadazol-2-amine is administered topically to the surface of the eye as a composition and separately applied periorbitally as a composition.

69. The method of any one of claims 66-68, wherein the composition is an aqueous solution, a non-aqueous solution, an oil solution, a gel, a suspension, an emulsion, a cream, or ointment, in liposomes, or in nanoparticles with or without co-incorporation of an siRNA or an antibody.

70. The method of any one of claims 66-69, wherein the composition is an aqueous solution.

71. The method of claim 70, wherein the aqueous solution comprises a polyoxyl castor oil.

72. The method of claim 71, wherein the polyoxyl castor oil is a polyethylene glycol (PEG)-ylated castor oil.

73. The method of claim 71 or 72, wherein the polyoxyl castor oil is polyoxyl 35 castor oil.

74. The method of claim 73, wherein the polyoxyl 35 castor oil is present in an amount of about 0.1% to about 5%, about 0.1% to about 10%, about 0.1% to about 15%, or about 0.1% to about 20% (w/w) of the composition.

75. The method of any one of claims 70-74, wherein the composition comprises an ocular surface lubricating agent.

76. The method of any one of claims 62-75, wherein the 4,5-dihydro-N-[4-[[4-(1-methylethoxy) phenyl] methyl] phenyl]-1H-imadazol-2-amine is topically applied by dropper, pump, spray, click pen or roller/reservoir device.

77. The method of any one of claims 62-76, wherein the 4,5-dihydro-N-[4-[[4-(1-methylethoxy) phenyl] methyl] phenyl]-1H-imadazol-2-amine is topically applied to the periorbital skin of at least one eye by brush, Q-tip, or spatula and where the application process may be preceded by using a graduated dropper, syringe, click pen or pipette.

78. The method of any one of claims 62-77, wherein the 4,5-dihydro-N-[4-[[4-(1-methylethoxy) phenyl] methyl] phenyl]-1H-imadazol-2-amine is administered to the patient four times per day, three times per day, twice per day, once per day, once every other day, once every three days, once every four days, or once every seven days.

79. The method of claim 78, wherein the 4,5-dihydro-N-[4-[[4-(1-methylethoxy) phenyl]methyl] phenyl]-1H-imadazol-2-amine is administered once per day.

80. The method of any one of claims 62-79, further comprising administering to the periorbital skin of the eye of the patient a topical pharmaceutical composition comprising an omega-3 fatty acid, or a pharmaceutically acceptable ester or salt thereof.

81. A pharmaceutical composition suitable for topical periorbital administration, comprising 4,5-dihydro-N-[4-[[4-(1-methylethoxy) phenyl] methyl] phenyl]-1H-imadazol-2-amine or a pharmaceutically acceptable ester or salt thereof.

82. The pharmaceutical composition of claim 81, wherein the composition is an aqueous solution, a non-aqueous solution, an oil solution, a gel, a suspension, an emulsion, a cream, an ointment, in liposomes or in nanoparticles with or without co-incorporation of an siRNA or an antibody.

83. The pharmaceutical composition of claim 81 or 82, wherein the composition is formulated as an oil solution.

84. The method of any one of claims 81-83, wherein the composition comprises an oil in an amount of about 1% to about 100% (w/w) of the composition.

85. The pharmaceutical composition of any one of claims 81-84 wherein the composition comprises an oil in an amount of at least about 90%, at least about 910%, at least about 92%, at least about 93%, at least about 94%, at least about 95%, at least about 96%, at least about 97%, at least about 98%, at least about 99%, at least about 99.5%, at least about 99.6%, at least about 99.7%, at least about 99.8%, at least about 99.9%, at least about 99.95%, at least about 99.96%, at least about 99.97%, at least about 99.98%, or at least about 99.99% (w/w) of the composition.

86. The pharmaceutical composition of any one of claims 82-85, wherein the oil is derived from a natural source.

87. The pharmaceutical composition of claim 86, wherein the oil is derived from plants, plant seeds, or nuts, or any combination thereof.

88. The pharmaceutical composition of any one of claims 83-87, wherein the oil comprises a medium-chain triglyceride.

89. The pharmaceutical composition of claim 88, wherein the medium-chain triglyceride comprise a mixture of C6, C8, C10 or C12 fatty acids.

90. The pharmaceutical composition of any one of claims 81-89, wherein the 4,5-dihydro-N-[4-[[4-(1-methylethoxy) phenyl] methyl] phenyl]-1H-imadazol-2-amine is present in an amount of from about 0.00015% to about 10% (w/w) of the composition.

91. The pharmaceutical composition of any one of claims 81-90, wherein the pharmaceutical composition is configured to dispense from about 0.5 microgram (μg) to about 5 milligrams (mg) of the 4,5-dihydro-N-[4-[[4-(1-methylethoxy)phenyl] methyl] phenyl]-1H-imadazol-2-amine per eye per administration.

92. The pharmaceutical composition of any one of claims 81-91, further comprising an emollient, a humectant, a thickening agent, a preservative, a penetration enhancer, or any combination thereof.

93. The pharmaceutical composition of any one of claims 81-92, further comprising an omega-3 fatty acid or a pharmaceutically acceptable ester or salt thereof.

94. A pharmaceutical composition suitable for topical ocular surface administration, comprising 4,5-dihydro-N-[4-[[4-(1-methylethoxy) phenyl] methyl] phenyl]-1H-imadazol-2-amine, or a pharmaceutically acceptable ester or salt thereof, and a polyoxyl castor oil.

95. The pharmaceutical composition of claim 94, wherein the polyoxyl castor oil is a polyethylene glycol (PEG)-ylated castor oil.

96. The pharmaceutical composition of claim 95, wherein the ratio of PEG to castor oil is from about 20:1 to about 50:1.

97. The pharmaceutical composition of any one of claims 94-96 wherein the polyoxyl castor oil is polyoxyl 35 castor oil.

98. The pharmaceutical composition of claim 97, wherein the polyoxyl 35 castor oil is present in an amount of about 0.1% to about 5%, about 0.1% to about 10%, about 0.1% to about 15%, or about 0.1% to about 20% (w/w) of the composition.

99. The pharmaceutical composition of claim 97 or 98, wherein the polyoxyl 35 castor oil is present in an amount of about 1% (w/w) of the composition.

100. The pharmaceutical composition of any one of claims 94-99, further comprising an ocular surface lubricating agent.

101. The pharmaceutical composition of claim 100, wherein the ocular surface lubricating agent is polyethylene glycol, propylene glycol, polyvinyl alcohol, castor oil or glycerol.

102. The pharmaceutical composition of claim 100 or 101, wherein the ocular surface lubricating agent is present in an amount of about 0.05% to about 2% (w/w) of the composition.

103. The pharmaceutical composition of any one of claims 94-102, further comprising a buffer.

104. The pharmaceutical composition of any one of claims 94-103, wherein the pharmaceutical composition has a pH of from about 6.5 to about 8.5.

105. The pharmaceutical composition of any one of claims 94-104, wherein the 4,5-dihydro-N-[4-[[4-(1-methylethoxy) phenyl] methyl] phenyl]-1H-imadazol-2-amine is present in an amount of from about 0.0001% to about 10% (w/w) of the composition.

106. A method of promoting ocular health or preventing or treating ocular disease in a subject, comprising administering to the periorbital skin of an eye the subject a topical pharmaceutical composition comprising an omega-3 fatty acid or a pharmaceutically acceptable ester or salt thereof.

107. The method of claim 106, wherein the omega-3 fatty acid is isolated from fish tissue.

108. The method of claim 106, wherein the omega-3 fatty acid is isolated from a plant source.

109. The method of any one of claims 106-108, wherein the omega-3 fatty acid comprises alpha-linolenic acid (ALA), eicosapentaenoic acid (EPA), docosahexaenoic acid (DHA), or any combination thereof.

110. The method of claim 109, wherein the omega-3 fatty acid comprises DHA.

111. The method of claim 109, wherein the omega-3 fatty acid comprises EPA.

112. The method of any one of claims 106-111, wherein the omega-3 fatty acid is administered in an amount of from about 0.1 mg to about 3000 mg, about 0.1 mg to about 1000 mg, about 0.1 mg to about 500 mg, about 0.1 mg to about 200 mg, or about 0.1 mg to about 100 mg.

113. The method of any one of claims 106-112, wherein the topical pharmaceutical composition is formulated as a cream, emulsion, ointment, or oil solution.

114. The method of any one of claims 106-113, wherein the topical pharmaceutical composition further comprises an emollient, a humectant, a thickening agent, a preservative, a penetration enhancer, an anti-oxidant, an odor masking agent, or any combination thereof.

115. The method of any one of claims 106-114, wherein the topical pharmaceutical composition further comprises a preservative.

116. The method of any one of claims 106-115, wherein the topical pharmaceutical composition further comprises 4,5-dihydro-N-[4-[[4-(1-methylethoxy) phenyl] methyl] phenyl]-1H-imadazol-2-amine, or a pharmaceutically acceptable ester or salt thereof.

117. The method of any one of claims 106-116, wherein the topical pharmaceutical composition is administered with a bottle with a roller ball, a click pen brush, a pump bottle, or an eye drop bottle and Q-tip.

118. The method of any one of claims 106-117, wherein promoting ocular health, preventing or treating ocular disease comprises treating or preventing dry eye disease and ocular discomfort, irritation, pain and stress, chemical burns, anterior segment dysgenesis, cataract, iritis, pterygium, keratoconjunctivitis, keratitis, conjunctivitis, keratoconus, ectatic disorders (including keratoglobus, pellucid marginal degeneration), Pseudophakic and aphakic bullous keratopathy, episcleritis, corneal ulceration, corneal dysplasia, corneal ulceration, Fuchs' endothelial dystrophy and other corneal dystrophies (including lattice, granular, macular, and map-dot fingerprint), ocular cicatricial pemphigoid, Stevens Johnson syndrome, acute and chronic uveitis (anterior uveitis, intermediate uveitis), trauma to the cornea, conjunctiva and anterior segment including iris trauma, penetrating ocular trauma, blepharitis, blepharospasm, chalazion, ptosis, coloboma, dermatochalasis, ectropion, entropion, trichiasis, stye, meibomianitis, Meibomian Gland Dysfunction, lacrimal gland obstruction, lacrimal gland obstruction, seborrheic keratitis, actinic keratitis, bacterial infection, or viral infection, age-related macular degeneration (wet and dry forms), dry and wet macular degeneration, lattice Degeneration, macular hole, macular pucker, lattice degeneration, retinal tear, retinal detachment, retinal artery occlusion, retinal vein occlusion, central retinal vein occlusion, intraocular tumors, pediatric, neonatal or Inherited retinal disorders, hereditary retinal dystrophies, geographic atrophy, retinitis pigmentosa (including Leber congenital amaurosis), cytomegalovirus (cmv) retinal infection, infectious retinitis, retinoblastoma, endophthalmitis, chorioretinitis, myopic macular degeneration, and normal-tension glaucoma, retinal degeneration in glaucoma; various retinopathies, including but not limited to diabetic retinopathy, retinopathy of prematurity, Sickel cell retinopathy, radiation/solar retinopathy, central serous retinopathy, hypertensive retinopathy, peripheral retinopathy and neuropathy; macular edema, retinal hemorrhage, diabetic macular edema, diabetic macular ischemia, geographic atrophy, Stargardt disease, refractive errors (myopia, hyperopia, and astigmatism), lymphatic malformations of the orbit (a.k.a. orbital lymphangiomas), thyroid eye disease (Graves' Eye Disease), or acute and chronic uveitis (including intermediate uveitis, posterior uveitis, panuveitis).

119. The method of any one of claims 106-118, wherein promoting ocular health, preventing or treating ocular disease comprises treating or preventing dry eye.

120. The method of any one of claims 106-119, wherein promoting ocular health, preventing or treating ocular disease comprises treating or preventing wet or dry age-related macular degeneration.

121. The method of any one of claims 106-120, wherein promoting ocular health, preventing or treating ocular disease comprises treating or preventing various retinopathies, including but not limited to diabetic retinopathy, retinopathy of prematurity.

122. The method of any one of claims 106-121, wherein the topical pharmaceutical composition is administered to the patient four times per day, three times per day, twice per day, once per day, once every other day, once every three days, once every four days, or once every seven days.

123. The method of any one of claims 106-122, wherein the method comprises administering the composition to the periorbital skin above the upper eyelid, below the lower eyelid, or both above the upper and below the lower eyelids.

124. The method of any one of claims 106-123, wherein administering the composition to the periorbital skin results in a tissue concentration of the omega-3 fatty acid of at least 110 micrograms/gram in the retina of the eye of the subject 30 minutes after administration greater than compared to baseline.

125. A pharmaceutical composition suitable for topical periorbital administration, comprising an omega-3 fatty acid, or a pharmaceutically acceptable ester or salt thereof, and a pharmaceutically acceptable excipient.

126. The composition of claim 125, wherein the omega-3 fatty acid is isolated from fish tissue.

127. The composition of claim 125, wherein the omega-3 fatty acid is isolated from a plant source.

128. The composition of any one of claims 125-127, wherein the omega-3 fatty acid comprises alpha-linolenic acid (ALA), eicosapentaenoic acid (EPA), docosahexaenoic acid (DHA), or any combination thereof.

129. The composition of claim 128, wherein the omega-3 fatty acid comprises DHA.

130. The composition of claim 128, wherein the omega-3 fatty acid comprises EPA.

131. The composition of any one of claims 125-130, wherein the omega-3 fatty acid is present in an amount of about 0.01% to about 100% (w/w) of the composition.

132. The composition of any one of claims 125-131, wherein the composition is formulated as a cream, emulsion, ointment, or oil solution.

133. The composition of any one of claims 125-132, wherein the composition further comprises an emollient, a humectant, a thickening agent, a preservative, a penetration enhancer, an anti-oxidant, an odor masking agent, or any combination thereof.

134. The composition of any one of claims 125-133, further comprising a preservative.

135. The composition of claim 134, wherein the preservative is vitamin E.

136. The composition of any one of claims 125-135, further comprising a fatty acid vehicle.

137. The composition of claim 136, wherein the fatty acid vehicle is present in an amount of from about 0.1% to about 99% of the composition.

138. The composition of claim 136 or 137, wherein the fatty acid vehicle is a C14 to C22 fatty acid.

139. The composition of any one of claims 136-138, wherein the fatty acid vehicle comprises linoleic acid.

140. The composition of any one of claims 125-135, further comprising an oil in an amount of about 1% to about 100% (w/w) of the composition.

141. The composition of claim 140, wherein the oil is derived from a natural source.

142. The composition of claim 140 or 141, wherein the oil is derived from plants, plant seeds, or nuts.

143. The composition of any one of claims 125-142, further comprising 4,5-dihydro-N-[4-[[4-(1-methylethoxy) phenyl] methyl] phenyl]-1H-imadazol-2-amine, or a pharmaceutically acceptable ester or salt thereof.

144. A method of treating a disease or disorder of the posterior of the eye in a patient suffering from the disease or disorder comprising administering a therapeutically effective amount of 4,5-dihydro-N-[4-[[4-(1-methylethoxy) phenyl] methyl] phenyl]-1H-imadazol-2-amine, or a pharmaceutically acceptable ester or salt thereof, to the exterior skin of the eyelid of an eye of the patient.

145. The method of claim 144, wherein the disease or disorder of the posterior of the eye comprises a retinal disease.

146. The method of claim 145, wherein the retinal disease comprises hemorrhage from the retinal or choroidal vasculature.

147. The method of claim 146, wherein the hemorrhage is caused by systemic hypertension, diabetes, fatty liver disease, obesity, shaken baby syndrome, head trauma, anemia, or leukemia.

148. The method of claim 145, wherein the retinal disease or disorder comprises plasma leakage from the retinal or choroidal vasculature.

149. The method of claim 148, wherein the plasma leakage is caused by systemic hypertension, diabetes, fatty liver disease, obesity, shaken baby syndrome, head trauma, anemia, or leukemia.

150. The method of claim 145, wherein the retinal disease or disorder comprises macular edema formation involving the retinal or choroidal vasculature.

151. The method of claim 144, wherein the disease or disorder of the posterior of the eye is age-related macular degeneration (wet and dry forms), dry and wet macular degeneration, lattice Degeneration, macular hole, macular pucker, lattice degeneration, retinal tear, retinal detachment, retinal artery occlusion, retinal vein occlusion, central retinal vein occlusion, intraocular tumors, pediatric, neonatal or Inherited retinal disorders, hereditary retinal dystrophies, geographic atrophy, retinitis pigmentosa (including Leber congenital amaurosis), cytomegalovirus (cmv) retinal infection, infectious retinitis, retinoblastoma, endophthalmitis, chorioretinitis, myopic macular degeneration, and normal-tension glaucoma, retinal degeneration in glaucoma; various retinopathies, including but not limited to diabetic retinopathy, retinopathy of prematurity, Sickel cell retinopathy, radiation/solar retinopathy, central serous retinopathy, hypertensive retinopathy, peripheral retinopathy and neuropathy, macular edema, retinal hemorrhage, diabetic macular edema, diabetic macular ischemia, geographic atrophy, Stargardt disease, uveitis (including intermediate uveitis, posterior uveitis, and panuveitis), or refractive errors (myopia, hyperopia, and astigmatism).

152. The method of any one of claims 145-151, wherein the retinal disease or disorder is age-related macular degeneration.

153. The method of claim 144, wherein the disease or disorder of the posterior of the eye is posterior uveitis.

154. The method of any one of claims 144-153, further comprising administering to the patient an additional therapeutic agent.

155. The method of claim 154, wherein the additional therapeutic agent is a VEGF antibody, a small molecule VEGF antagonist, a siRNA targeting a VEGF receptor, a TNFα antibody, a small molecule TNFα receptor antagonist, a siRNA targeting the TNFα receptor, an inflammatory cytokine receptor antagonist, an antibody against an inflammatory cytokine, a tyrosine kinase inhibitor, a serine/threonine-protein kinase inhibitors, a kinase inhibitor, a steroidal anti-inflammatory agent, a non-steroidal anti-inflammatory agent, an immunosuppressant, an anti-cholinergic agent, thalidomide, a prostaglandin receptor antagonist, a neuroprotective agent, a neurotrophic agent, a neuro-regenerative agent, an ocular hypotensive agent, an antibiotics, an antiviral agent, a complement inhibitor, an interleukin receptor inhibitor, a leukotriene receptor inhibitor, an inhibitor of tumorigenesis and development, an angiogenesis inhibitor, or agents with anti-oxidation or anti-microvascular leakage properties.

156. The method of any one of claims 144-155, wherein the 4,5-dihydro-N-[4-[[4-(1-methylethoxy) phenyl] methyl] phenyl]-1H-imadazol-2-amine is administered prophylactically, as an emergency intervention, or as required to achieve the desired remedial effects.

157. The method of any one of claims 144-156, wherein the 4,5-dihydro-N-[4-[[4-(1-methylethoxy) phenyl] methyl] phenyl]-1H-imadazol-2-amine is administered as a composition.

158. The method of claim 146, wherein the 4,5-dihydro-N-[4-[[4-(1-methylethoxy) phenyl]methyl] phenyl]-1H-imadazol-2-amine is present in an amount of about 0.0001% to about 10% (w/w) of the composition.

159. The method of claim 157 or 158, wherein the composition is an aqueous solution, a non-aqueous solution, an oil solution, a gel, a suspension, an emulsion, a lotion, a cream, or an ointment.

160. The method of any one of claims 157-159, wherein the composition is an ointment.

161. The method of claim 160, wherein the ointment comprises petrolatum, beeswax, or cocoa butter.

162. The method of claim 160 or 161, wherein the ointment comprises petrolatum and medium-chain triglycerides.

163. The method of claim 162, wherein the medium-chain triglycerides comprise a mixture of C6, C8, C10 and C12 fatty acids.

164. The method of claim 162 or 163, wherein the medium-chain triglycerides comprise a mixture of caprylic acid and capric acid.

165. The method of any one of claims 160-164, wherein the ointment comprises petrolatum and medium-chain triglyceride in the ratio of about 1:1 (v/v), about 2:1 (v/v), about 3:1 (v/v), about 4:1 (v/v), about 5:1 (v/v), or about 6:1 (v/v).

166. The method of claim 165, wherein the ointment comprises petrolatum and medium-chain triglyceride in the ratio of about 4:1 (v/v).

167. The method of any one of claims 157-159, wherein the composition is an aqueous solution.

168. The method of claim 167, wherein the aqueous solution comprises a polyoxyl castor oil.

169. The method of claim 168, wherein the polyoxyl castor oil is a polyethylene glycol (PEG)-ylated castor oil.

170. The method of claim 168 or 169, wherein the polyoxyl castor oil is polyoxyl 35 castor oil.

171. The method of claim 170, wherein the polyoxyl 35 castor oil is present in an amount of about 0.1% to about 5%, about 0.1% to about 10%, about 0.1% to about 15%, or about 0.1% to about 20% (w/w) of the composition.

172. The method of any one of claims 167-171, wherein the composition comprises an ocular surface lubricating agent.

173. The method of any one of claims 144-172, wherein the 4,5-dihydro-N-[4-[[4-(1-methylethoxy) phenyl] methyl] phenyl]-1H-imadazol-2-amine is applied to the exterior skin of the eyelid of an eye of the patient by dropper, pump, spray, click pen or roller/reservoir device.

174. The method of any one of claims 144-172, wherein the 4,5-dihydro-N-[4-[[4-(1-methylethoxy) phenyl] methyl] phenyl]-1H-imadazol-2-amine is applied to the exterior skin of the eyelid of an eye of the patient by brush, Q-tip, or spatula and where the application process is optionally preceded by using a graduated dropper, syringe, click pen or pipette.

175. The method any one of claims 144-174 wherein eyelid skin penetration is assisted by tape-stripping, microdermabrasion, solvent, pulsed laser, iontophoresis, or combinations thereof.

176. The method of any one of claims 144-175, wherein the 4,5-dihydro-N-[4-[[4-(1-methylethoxy) phenyl] methyl] phenyl]-1H-imadazol-2-amine is administered to the eyelid skin of each eye of the patient four times per day, three times per day, twice per day, once per day, once every other day, once every three days, once every four days, or once every seven days.

177. The method of claim 176, wherein the 4,5-dihydro-N-[4-[[4-(1-methylethoxy) phenyl]methyl] phenyl]-1H-imadazol-2-amine is administered once per day.

178. The method of claim 154, wherein the additional therapeutic agent is an omega-3 fatty acid or a pharmaceutically acceptable ester or salt thereof.

179. The method of claim 178, wherein the 4,5-dihydro-N-[4-[[4-(1-methylethoxy) phenyl]methyl] phenyl]-1H-imadazol-2-amine and the omega-3 fatty acid or a pharmaceutically acceptable ester or salt thereof are formulated and administered as a single composition.

180. An active ingredient formulated for topical administration to the periorbital skin of a patient, for use in the manufacture of a medicament for treating a disease or disorder of the posterior of the eye, wherein the formulation delivers a therapeutically effective amount of said active ingredient formulated for topical administration to the periorbital skin of a patient to the posterior of the eye.

181. The formulation of claim 180, wherein the active ingredient has a molecular weight of less than or equal to 1000 Da.

182. The formulation of claim 180, wherein the active ingredient has a molecular weight of 200-500 Da.

183. The formulation of claim 180, wherein 1 milliliter to 10 milliliters of formulation are topically applied to the periorbital skin of one eye of a patient per use, wherein the active ingredient is topically applied using an eye pad.

184. The formulation of claim 180, wherein 3 microliters to 100 microliters of formulation are topically applied directly to the periorbital skin of one eye of a patient per use.

185. The formulation of claim 180, wherein 0.01 milligrams to 10 grams of active ingredient are topically applied to the periorbital skin of one eye of a patient per use, wherein the active ingredient is topically applied using an eye pad.

186. The formulation of claim 180, wherein 0.01 milligrams to 100 milligrams of active ingredient are topically applied directly to the periorbital skin of one eye of a patient per use.

187. The formulation of claim 180, further comprising an oil in an amount of about 1% to about 100% (w/w) of the composition.

188. The formulation of claim 187, wherein the oil is derived from a natural source.

189. The formulation of claim 187 or 188, wherein the oil is derived from plants, plant seeds, or nuts.

190. The formulation of claim 187, wherein the oil comprises a medium-chain triglyceride.

191. The formulation of claim 189, wherein the oil comprises soybean oil.

192. The formulation of claim 180, wherein the active ingredient is an omega-3 fatty acid or a pharmaceutically acceptable ester or salt thereof.

193. The formulation of claim 192, wherein the omega-3 fatty acid or pharmaceutically acceptable ester or salt thereof comprises alpha-linolenic acid (ALA), eicosapentaenoic acid (EPA), docosahexaenoic acid (DHA), or any combination thereof.

194. The formulation of claim 193, wherein the omega-3 fatty acid or pharmaceutically acceptable ester or salt thereof comprises DHA.

195. The formulation of claim 193, wherein the omega-3 fatty acid or pharmaceutically acceptable ester or salt thereof comprises EPA.

196. The formulation of claim 192, wherein the omega-3 fatty acid or pharmaceutically acceptable ester or salt thereof is present in an amount of about 0.01% to about 100% (w/w) of the composition

197. The formulation of claim 192, wherein administering 6.7 mg of the formulation results in a tissue concentration of the omega-3 fatty acid 30 in the posterior of the eye of the patient 30 minutes after administration of about 110 micrograms/gram greater than compared to baseline.

198. The formulation of claim 192, further comprising 4,5-dihydro-N-[4-[[4-(1-methylethoxy) phenyl] methyl] phenyl]-1H-imadazol-2-amine or a pharmaceutically acceptable ester or salt thereof.

199. The formulation of claim 180, wherein the expected range of active ingredient detectable in the posterior tissue of the eye is about 0.1 μg to about 1600 μg per gram of posterior tissue.

200. The formulation of claim 180, wherein the active ingredient is administered to the periorbital skin of each eye of the patient four times per day, three times per day, twice per day, or once per day.

201. The formulation of claim 180, wherein the active ingredient is 4,5-dihydro-N-[4-[[4-(1-methylethoxy) phenyl] methyl] phenyl]-1H-imadazol-2-amine or a pharmaceutically acceptable ester or salt thereof.

202. The formulation of claim 202, wherein said 4,5-dihydro-N-[4-[[4-(1-methylethoxy) phenyl] methyl] phenyl]-1H-imadazol-2-amine is present in an amount of from about 0.00005% to about 10% (w/w) of the composition.

203. The formulation of claim 202, further comprising an omega-3 fatty acid or pharmaceutically acceptable ester or salt thereof.
